(19) United States
(12) Patent Application Publication
(10) Pub. No.: US 2016/0112684 A1
(43) Pub. Date: Apr. 21, 2016

(54) Spectroscopic Finger Ring for Compositional Analysis of Food or Other Environmental Objects

(71) Applicant: Robert A. Connor, Forest Lake, MN (US)

(72) Inventor: Robert A. Connor, Forest Lake, MN (US)

(73) Assignee: Medibotics LLC, Forest Lake, MN (US)

(21) Appl. No.: 14/948,308

(22) Filed: Nov. 21, 2015

Related U.S. Application Data
(63) Continuation-in-part of application No. 13/901,099, filed on May 23, 2013, now Pat. No. 9,254,099.
Application No. 13/901,099 is a continuation-in-part of application No. 14/132,292, filed on Dec. 18, 2013.
Application No. 14/132,292 is a continuation-in-part of application No. 14/449,387, filed on Aug. 1, 2014.

(51) Int. Cl. H04N 7/18; G01J 3/28; G06K 9/00; G06K 9/22; G06T 7/00; G01N 33/02; G01J 3/02

(52) U.S. Cl. CPC H04N 7/185; G01N 33/02; G01J 3/28; G01J 3/0202; G01J 3/0272; G06K 9/228; G06T 7/0004; G06K 9/00771; G06K 2209/17; G06T 2207/30128

(57) ABSTRACT

This invention is a wearable spectroscopic device, such as a spectroscopic finger ring, for compositional analysis of food or other environmental objects. This device can project light as a fiducial marker to better estimate object size. This device can include a laser pointer which is directed toward an object to guide spectroscopic analysis of the object. Advantages over hand-held spectroscopic sensors include: convenience; subtlety of use; activation based on monitoring of body motion and/or hand gestures; and continuous proximity to hand-held food during eating.

### CROSS-REFERENCE TO RELATED APPLICATIONS

This patent application is: (a) a continuation in part of U.S. patent application Ser. No. 13/901,099 by Robert A. Connor entitled “Smart Watch and Food-Imaging Member for Monitoring Food Consumption” filed on May 23, 2013; (b) a continuation in part of U.S. patent application Ser. No. 14/132,292 by Robert A. Connor entitled “Caloric Intake Measuring System using Spectroscopic and 3D Imaging Analysis” filed on Dec. 18, 2013, whose specification claimed divisional status relative to U.S. patent application Ser. No. 13/901,099 by Robert A. Connor entitled “Smart Watch and Food-Imaging Member for Monitoring Food Consumption” filed on May 23, 2013; and (c) a continuation in part of U.S. patent application Ser. No. 14/449,387 by Robert A. Connor entitled “Wearable Imaging Member and Spectroscopic Optical Sensor for Food Identification and Nutrition Modification” filed on Aug. 1, 2014, whose specification claimed continuation status relative to U.S. patent application Ser. No. 13/901,099 by Robert A. Connor entitled “Smart Watch and Food-Imaging Member for Monitoring Food Consumption” filed on May 23, 2013. The entire contents of these related applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to wearable technology for spectroscopic analysis of the composition of food or other environmental objects.

### INTRODUCTION

The United States population has some of the highest prevalence rates of obese and overweight people in the world. Further, these rates have increased dramatically during recent decades. In the late 1990's, around one in five Americans was obese. Today, that figure has increased to around one in three. It is estimated that around one in five American children is now obese. The prevalence of Americans who are generally overweight is estimated to be as high as two out of three. Despite the considerable effort that has been focused on developing new approaches for preventing and treating obesity, the problem is growing. There remains a serious unmet need for new ways to help people to moderate their consumption of unhealthy food, better manage their energy balance, and lose weight in a healthy and sustainable manner.

Obesity is a complex disorder with multiple interacting causal factors including genetic factors, environmental factors, and behavioral factors. A person's behavioral factors include the person's caloric intake (the types and quantities of food which the person consumes) and caloric expenditure (the calories that the person burns in regular activities and exercise). Energy balance is the net difference between caloric intake and caloric expenditure. Other factors being equal, energy balance surplus (caloric intake greater than caloric expenditure) causes weight gain and energy balance deficit (caloric intake less than caloric expenditure) causes weight loss.

Since many factors contribute to obesity, good approaches to weight management are comprehensive in nature. Proper nutrition and management of caloric intake are key parts of a comprehensive approach to weight management. Consumption of “junk food” that is high in simple sugars and saturated fats has increased dramatically during the past couple decades, particularly in the United States. This has contributed significantly to the obesity epidemic. For many people, relying on willpower and dieting is not sufficient to moderate their consumption of unhealthy “junk food.” The results are dire consequences for their health and well-being.

The invention that is disclosed herein directly addresses this problem by helping a person to monitor their nutritional intake. The invention that is disclosed herein is an innovative technology that can be a key part of a comprehensive system that helps a person to reduce their consumption of unhealthy food, to better manage their energy balance, and to lose weight in a healthy and sustainable manner. This invention is a wearable spectroscopic device for compositional analysis of food. In an example, this invention can be embodied in a spectroscopic finger ring. This invention can also be useful for applications other than monitoring nutritional intake when convenient, gesture-directed compositional analysis of environmental objects is needed.

### REVIEW OF THE RELATED ART

Application WO 2010/070645 by Einav et al. entitled “Method and System for Monitoring Eating Habits” discloses an apparatus for monitoring eating patterns which can include a spectrometer for detecting nutritious properties of a bite of food.

U.S. Pat. No. 8,355,875 by Hyde et al. entitled “Food Content Detector” discloses a utensil for portioning a foodstuff into first and second portions which can include a spectroscopy sensor.

U.S. patent application 20140061486 by Bao et al. entitled “Spectrometer Devices” discloses a spectrometer including a plurality of semiconductor nanocrystals which can serve as a personal UV exposure tracking device. Other applications include a smartphone or medical device wherein a semiconductor nanocrystal spectrometer is integrated.

SCiO is a molecular sensor which has been disclosed by Consumer Physics which appears to use near-infrared spectroscopy to analyze the composition of nearby objects and may be used to analyze the composition of food. U.S. patent 20140320858 by Goldring et al. (who appears to be part of the Consumer Physics team) is entitled “Low-Cost Spectrometry System for End-User Food Analysis” and discloses a compact spectrometer that can be used in mobile devices such as cellular telephones.

U.S. patent application 20140347491 by Connor entitled “Smart Watch and Food-Imaging Member for Monitoring Food Consumption” discloses a device and system for monitoring a person's food consumption comprising: a wearable sensor that automatically collects data to detect probable eating events; an imaging member that is used by the person to take pictures of food wherein the person is prompted to take pictures of food when an eating event is detected by the wearable sensor; and a data analysis component that analyzes these food pictures to estimate the types and amounts of foods, ingredients, nutrients, and/or calories that are consumed by the person.

TellSpec, which raised funds via Indiegogo in 2014, is intended to be a hand-held device which uses spectroscopy to measure the nutrient composition of food. Their U.S. patent application 20150036138 by Watson et al. entitled “Analyzing and Correlating Spectra, Identifying Samples and Their Ingredients, and Displaying Related Personalized Information” describes obtaining two spectra from the same sample under two different conditions at about the same time for comparison. Further, this application describes how computing correlations between data related to food and ingredient consumption by users and personal log data (and user entered feedback, user interaction data or personal information related to those users) can be used to detect foods to which a user may be allergic.

U.S. patent application 20150126873 by Connor entitled “Wearable Spectroscopy Sensor to Measure Food Consumption” discloses a wearable device to measure a person's consumption of selected types of food, ingredients, or nutrients comprising: a housing that is configured to be worn on the person's wrist, arm, hand, or finger; a spectroscopy sensor that collects data concerning light energy reflected from the person's body and/or absorbed by the person's body, wherein this data is used to measure the person's consumption of selected types of food, ingredients, or nutrients; a data processing unit; and a power source.

U.S. patent application 20150148632 by Benaron entitled “Calorie Monitoring Sensor and Method for Cell Phones, Smart Watches, Occupancy Sensors, and Wearables” discloses a sensor for calorie monitoring in mobile devices, wearables, security, illumination, photography, and other devices and systems which uses an optional phosphor-coated broadband white LED to produce broadband light, which is then transmitted along with any ambient light to a target such as the ear, face, or wrist of a living subject. Calorie monitoring systems incorporating the sensor as well as methods are also disclosed.

U.S. patent application 20150148636 by Benaron entitled “Ambient Light Method for Cell Phones, Smart Watches, Occupancy Sensors, and Wearables” discloses a sensor for respiratory and metabolic monitoring in mobile devices, wearables, security, illumination, photography, and other devices and systems that uses a broadband ambient light. The sensor can provide identifying features of type or status of a tissue target, such calories used or ingested.

U.S. patent application 20150168365 by Connor entitled “Caloric Intake Measuring System Using Spectroscopic and 3D Imaging Analysis” discloses a caloric intake measuring system comprising: a spectroscopic sensor that collects data concerning light that is absorbed by or reflected from food, wherein this food is to be consumed by a person, and wherein this data is used to estimate the composition of this food; and an imaging device that takes images of this food from different angles, wherein these images from different angles are used to estimate the quantity of this food.

U.S. patent application 20150302160 by Muthukumar et al. entitled “Method and Apparatus for Monitoring Diet and Activity” discloses a method and apparatus including a camera and spectroscopy module for determining food types and amounts.

### SUMMARY AND ADVANTAGES OF THE INVENTION

This invention is a wearable spectroscopic device for compositional analysis of food (or other environmental objects) which projects a beam of light that serves as a fiducial marker for image analysis to better estimate the size of the food (or other object) and/or which includes a laser pointer which the wearer directs toward the food (or other object) to guide spectroscopic analysis of the food (or other object). Such a wearable spectroscopic device provides advantages over hand-held or counter-top spectroscopic devices. These advantages include: greater convenience; more subtle use; activation and control based on continuous monitoring of body motion and/or hand gestures; and continuous proximity of the spectroscopic sensor to a hand-held object during eating.

In an example, this invention can be embodied in a wearable device for food identification and quantification comprising: (a) a camera which takes pictures of nearby food, wherein these food pictures are analyzed in order to identify the types and quantities of food; (b) a light-emitting member which projects a light-based fiducial marker on, or in proximity to, the nearby food as an aid in estimating food size; (c) a spectroscopic optical sensor, wherein this spectroscopic optical sensor collects data concerning light that is reflected from, or has passed, through the nearby food and wherein this data is analyzed to identify the types of food, the types of ingredients in the food, and/or the types of nutrients in the food; (d) an attachment mechanism, wherein this attachment mechanism is configured to hold the camera, the light-emitting member, and the spectroscopic optical sensor in close proximity to the surface of a person's body; and (e) an image-analyzing member which analyzes the food pictures.

In an example, this invention can be embodied in a wearable spectroscopic device for compositional analysis of environmental objects comprising: a finger ring, wherein this finger ring further comprises: (a) a finger-encircling portion, wherein this finger-encircling portion is configured to encircle at least 70% of the circumference of a person's finger, wherein this finger-encircling portion has an interior surface which is configured to face toward the surface of the person's finger when worn, wherein this finger-encircling portion has a central proximal-to-distal axis which is defined as the straight line which most closely fits a proximal-to-distal series of centroids of cross-sections of the interior surface, and wherein proximal is defined as being closer to a person's elbow and distal is defined as being further from a person's elbow when the person's arm, hand, and fingers are fully extended; (b) a light-emitting member which projects a beam of light along a proximal-to-distal vector toward an object in the person's environment, wherein this vector, or a virtual extension of this vector, is either parallel to the central proximal-to-distal axis or intersects a line which is parallel to the central proximal-to-distal axis forming a distally-opening angle whose absolute value is less than 45 degrees; and (c) a spectroscopic optical sensor which collects data concerning the spectrum of light which is reflected from, or has passed through, the object in the person's environment, wherein data from the spectroscopic optical sensor is used to analyze the composition of this object, and wherein this spectroscopic optic sensor is selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

In an example, a beam of light projected by a light-emitting member can be near-infrared light, infrared light, or ultra-violet light. In an example, a beam of light projected by a light-emitting member can be white light and/or reflected ambient light. In an example, a beam of light projected by a light-emitting member can be coherent light. In an example, this device can further comprise a laser pointer which is moved by a person in order to direct a visible beam of coherent light toward an object in the environment in order to guide, direct, select, adjust, and/or trigger spectroscopic analysis of this object.

In an example, the vector of a beam of light projected by a light-emitting member can be automatically changed in response to detection of an object in the environment and/or changes in the location of an object in the environment. In an example, the vector of a beam of light projected by a light-emitting member can be selected in order to direct reflected light back to a spectroscopic optical sensor from an object at a selected focal distance, wherein this selected focal distance can be selected based on detection of the object at the selected distance, and wherein measurement of the object's distance can be based on image analysis, reflection of light energy, reflection of radio waves, reflection of sonic energy, and/or gesture recognition. In an example, the vector of a beam of light emitted by a light-emitting member can be varied in order to scan for objects in the environment at different distances and/or to scan a larger portion of the surface of an object in the environment.

In an example, this invention can further comprise a data processing unit which at least partially processes data from the spectroscopic optical sensor. In an example, this invention can further comprise a wireless data transmitter through which the device is in wireless communication with another wearable device and/or a remote computer and wherein information concerning the composition of an environmental object is displayed by the other wearable device and/or remote computer. In an example, this invention can further comprise a motion sensor. Motion patterns can be analyzed in order to trigger or adjust the parameters of a spectroscopic scan of an object in the environment. In an example, a spectroscopic scan can be triggered when motion patterns indicate that a person is eating. In an example, this invention can perform multiple spectroscopic scans, at different times, while a person is eating in order to better analyze the overall composition of food with different internal layers and/or a non-uniform ingredient structure.

### BRIEF INTRODUCTION TO THE FIGURES

FIGS. 1 through 30 show various examples of how this invention can be embodied in a wearable device for spectroscopic analysis of food or other environmental objects. However, these figures do not limit the full generalizability of the claims.

FIGS. 1 through 4 show an example of a device to monitor a person's food consumption comprising a smart watch (with a motion sensor) to detect eating events and a smart spoon (with a built-in chemical composition sensor), wherein the person is prompted to use the smart spoon to eat food when the smart watch detects an eating event.

FIGS. 5 through 8 show an example of a device to monitor a person's food consumption comprising a smart watch (with a motion sensor) to detect eating events and a smart spoon (with a built-in camera), wherein the person is prompted to use the smart spoon to take pictures of food when the smart watch detects an eating event.

FIGS. 9 through 12 show an example of a device to monitor a person's food consumption comprising a smart watch (with a motion sensor) to detect eating events and a smart phone (with a built-in camera), wherein the person is prompted to use the smart phone to take pictures of food when the smart watch detects an eating event.

FIGS. 13 through 15 show an example of a device to monitor a person's food consumption comprising a smart necklace (with a microphone) to detect eating events and a smart phone (with a built-in camera), wherein the person is prompted to use the smart phone to take pictures of food when the smart necklace detects an eating event.

FIGS. 16 through 18 show an example of a device to monitor a person's food consumption comprising a smart necklace (with a microphone) to detect eating events and a smart spoon (with a built-in chemical composition sensor), wherein the person is prompted to use the smart spoon to eat food when the smart necklace detects an eating event.

FIG. 19 shows an example of a wearable device for food identification and quantification comprising an imaging member (e.g. camera), an optical sensor (e.g. spectroscopic optical sensor), an attachment mechanism (e.g. wrist band), and an image-analyzing member (e.g. data control unit), wherein the imaging member and optical sensor are on the anterior/palmar/lower side of a person's wrist.

FIG. 20 shows an example that is like the example in FIG. 19 except that FIG. 20 further comprises a projected light-based fiducial marker.

FIG. 21 shows an example of a wearable device for food identification and quantification comprising an imaging member (e.g. camera), an optical sensor (e.g. spectroscopic optical sensor), an attachment mechanism (e.g. wrist band), and an image-analyzing member (e.g. data control unit), wherein the imaging member and optical sensor are on the lateral/narrow side of a person's wrist.

FIG. 22 shows an example that is similar to the example in FIG. 21 except that FIG. 22 further comprises a computer-to-human interface that is an implanted substance-releasing device that releases an absorption-reducing substance into the person's stomach.

FIG. 23 shows an example that is similar to the example in FIG. 21 except that FIG. 23 further comprises a computer-to-human interface that is an implanted electromagnetic energy emitter that delivers electromagnetic energy to a portion of the person's gastrointestinal tract and/or to nerves which innervate that portion.

FIG. 24 shows an example that is similar to the example in FIG. 21 except that FIG. 24 further comprises a computer-to-human interface that is an implanted electromagnetic energy emitter that delivers electromagnetic energy to nerves which innervate a person's tongue and/or nasal passages.

FIG. 25 shows an example that is similar to the example in FIG. 21 except that FIG. 25 further comprises a computer-to-human interface that is an implanted substance-releasing device that releases a taste and/or smell modifying substance into a person's oral cavity and/or nasal passages.

FIG. 26 shows an example that is similar to the example in FIG. 21 except that FIG. 26 further comprises a computer-to-human interface that is an implanted gastrointestinal constriction device.

FIG. 27 shows an example that is similar to the example in FIG. 21 except that FIG. 27 further comprises eyewear and a virtually-displayed image.

FIG. 28 shows an example that is similar to the example in FIG. 21 except that FIG. 28 further comprises an audio message to the person wearing the device.

FIGS. 29 and 30 show an example of a spectroscopic finger ring for analyzing the composition of food or other environmental objects.

### DETAILED DESCRIPTION OF THE FIGURES

### Device for Food Identification and Quantification:

In an example, a wearable device for food identification and quantification can comprise: at least one imaging member, wherein this imaging member takes pictures and/or records images of nearby food, and wherein these food pictures and/or images are automatically analyzed to identify the types and quantities of food; an optical sensor, wherein this optical sensor collects data concerning light that is transmitted through or reflected from nearby food, and wherein this data is automatically analyzed to identify the types of food, the types of ingredients in the food, and/or the types of nutrients in the food; one or more attachment mechanisms, wherein these one or more attachment mechanisms are configured to hold the imaging member and the optical sensor in close proximity to the surface of a person's body; and an image-analyzing member which automatically analyzes food pictures and/or images.

With respect to the imaging member, a device, system, or method for measuring types of food, ingredients, and/or nutrients can include a camera or other picture-taking device that takes pictures of food. In an example, a camera can be positioned on a person's wrist at a location from which it takes pictures along an imaging vector that is directed generally downward toward a reachable food source. In an example, a device, system, or method for measuring types of food, ingredients, and/or nutrients can take pictures of food using a device selected from the group consisting of: smart watch, smart bracelet, fitness watch, fitness bracelet, watch phone, bracelet phone, wrist band, or other wrist-worn device; arm bracelet; and smart ring or finger ring.

With respect to analyzing pictures or images of nearby food, one or more methods to analyze pictures or images in order to estimate types and quantities of food can be selected from the group consisting of: pattern recognition; food recognition; word recognition; logo recognition; bar code recognition; face recognition; gesture recognition; and human motion recognition. In various examples, a picture or image of a person's mouth and/or a reachable food source can be analyzed with one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling.

In an example, a device can measure a person's consumption of at least one type of food, ingredient, or nutrient. In an example, a device can identify and track in an entirely automatic manner the types and amounts of foods, ingredients, or nutrients that a person consumes. In an example, such identification can occur in a partially-automatic manner in which there is interaction between automated and human identification methods. In an example, identification (from pictures of food) of the types and quantities of food, ingredients, or nutrients that a person consumes can be a combination of, or interaction between, automated food identification methods and human-based food identification methods. In various examples, automatic identification of food types and quantities can be based on: color and texture analysis; image segmentation; image pattern recognition; volumetric analysis based on a fiducial marker or other object of known size; and/or three-dimensional modeling based on pictures from multiple perspectives.

The term “food” is broadly defined herein to include liquid nourishment, such as beverages, in addition to solid food. Food consumption is broadly defined to include consumption of liquid beverages and gelatinous food as well as consumption of solid food. In an example, nearby food can also be referred to as a “reachable food source” and can be defined as a source of food that a person can access and from which they can bring a piece (or portion) of food to their mouth by moving their arm and hand. In an example, nearby food can be selected from the group consisting of: food on a plate, food in a bowl, food in a glass, food in a cup, food in a bottle, food in a can, food in a package, food in a container, food in a wrapper, food in a bag, food in a box, food on a table, food on a counter, food on a shelf, and food in a refrigerator.

With respect to different types of food, a device, system, or method for measuring types of food, ingredients, and/or nutrients should be able to differentiate between healthy foods vs unhealthy foods. This requires the ability to identify consumption of selected types of food, ingredients, and/or nutrients, as well as estimate the amounts of such consumption. It also requires selection of certain types and/or amounts of food, ingredients, and/or nutrients as healthy vs. unhealthy. In an example, a food-identifying device can selectively detect one or more types of unhealthy food, wherein unhealthy food is selected from the group consisting of: food that is high in simple carbohydrates; food that is high in simple sugars; food that is high in saturated or trans fat; fried food; food that is high in Low Density Lipoprotein (LDL); and food that is high in sodium.

In an example, a device can identify and quantify one or more selected types of food, ingredients, and/or nutrients selected from the group consisting of: a specific type of carbohydrate, a class of carbohydrates, or all carbohydrates; a specific type of sugar, a class of sugars, or all sugars; a specific type of fat, a class of fats, or all fats; a specific type of cholesterol, a class of cholesterols, or all cholesterols; a specific type of protein, a class of proteins, or all proteins; a specific type of fiber, a class of fiber, or all fiber; a specific sodium compound, a class of sodium compounds, and all sodium compounds; high-carbohydrate food, high-sugar food, high-fat food, fried food, high-cholesterol food, high-protein food, high-fiber food, and high-sodium food.

In an example, a device can identify and quantify a person's consumption of food that is high in simple carbohydrates. In an example, a device can identify and quantify a person's consumption of food that is high in simple sugars. In an example, a device can identify and quantify a person's consumption of food that is high in saturated fats. In an example, a device can identify and quantify a person's consumption of food that is high in trans fats. In an example, a device can identify and quantify a person's consumption of food that is high in Low Density Lipoprotein (LDL). In an example, a device can identify and quantify a person's consumption of food that is high in sodium.

In an example, a device can measure a person's consumption of food wherein a high proportion of its calories comes from simple carbohydrates. In an example, a device can measure a person's consumption of food wherein a high proportion of its calories comes from simple sugars. In an example, a device can measure a person's consumption of food wherein a high proportion of its calories comes from saturated fats. In an example, a device can measure a person's consumption of food wherein a high proportion of its calories comes from trans fats. In an example, a device can measure a person's consumption of food wherein a high proportion of its calories comes from Low Density Lipoprotein (LDL). In an example, a device can measure a person's consumption of food wherein a high proportion of its weight or volume is comprised of sodium compounds.

In an example, a device can measure a person's consumption of one or more selected types of food, ingredients, and/or nutrients selected from the group consisting of: simple carbohydrates, simple sugars, saturated fat, trans fat, Low Density Lipoprotein (LDL), and salt. In an example, a device can measure a person's consumption of simple carbohydrates. In an example, a device can measure a person's consumption of simple sugars. In an example, a device can measure a person's consumption of saturated fats. In an example, a device can measure a person's consumption of trans fats. In an example, a device can measure a person's consumption of Low Density Lipoprotein (LDL). In an example, a device can measure a person's consumption of sodium.

In an example, a device can identify and quantify one or more selected types of food, ingredients, and/or nutrients selected from the group consisting of: amino acid or protein (a selected type or general class), carbohydrate (a selected type or general class, such as single carbohydrates or complex carbohydrates), cholesterol (a selected type or class, such as HDL or LDL), dairy products (a selected type or general class), fat (a selected type or general class, such as unsaturated fat, saturated fat, or trans fat), fiber (a selected type or class, such as insoluble fiber or soluble fiber), mineral (a selected type), vitamin (a selected type), nuts (a selected type or general class, such as peanuts), sodium compounds (a selected type or general class), sugar (a selected type or general class, such as glucose), and water. In an example, food can be classified into general categories such as fruits, vegetables, or meat.

In an example, a device can identify one or more potential food allergens, toxins, or other substances selected from the group consisting of: ground nuts, tree nuts, dairy products, shell fish, eggs, gluten, pesticides, animal hormones, and antibiotics. In an example, a device can identify one or more types of food whose consumption is prohibited or discouraged for religious, moral, and/or cultural reasons, such as pork or meat products of any kind. In an example, a device for measuring nutrient consumption can track the quantities of selected chemicals that a person consumes via food consumption. In various examples, these consumed chemicals can be selected from the group consisting of carbon, hydrogen, nitrogen, oxygen, phosphorus, and sulfur.

In an example, a device can identify and quantify one or more types of food, ingredients, and/or nutrients selected from the group consisting of: a selected food, ingredient, or nutrient that has been designated as unhealthy by a health care professional organization or by a specific health care provider for a specific person; a selected substance that has been identified as an allergen for a specific person; peanuts, shellfish, or dairy products; a selected substance that has been identified as being addictive for a specific person; alcohol; a vitamin or mineral; vitamin A, vitamin B1, thiamin, vitamin B12, cyanocobalamin, vitamin B2, riboflavin, vitamin C, ascorbic acid, vitamin D, vitamin E, calcium, copper, iodine, iron, magnesium, manganese, niacin, pantothenic acid, phosphorus, potassium, riboflavin, thiamin, and zinc; a selected type of carbohydrate, class of carbohydrates, or all carbohydrates; a selected type of sugar, class of sugars, or all sugars; simple carbohydrates, complex carbohydrates; simple sugars, complex sugars, monosaccharides, glucose, fructose, oligosaccharides, polysaccharides, starch, glycogen, disaccharides, sucrose, lactose, starch, sugar, dextrose, disaccharide, fructose, galactose, glucose, lactose, maltose, monosaccharide, processed sugars, raw sugars, and sucrose; a selected type of fat, class of fats, or all fats; fatty acids, monounsaturated fat, polyunsaturated fat, saturated fat, trans fat, and unsaturated fat; a selected type of cholesterol, a class of cholesterols, or all cholesterols; Low Density Lipoprotein (LDL), High Density Lipoprotein (HDL), Very Low Density Lipoprotein (VLDL), and triglycerides; a selected type of protein, a class of proteins, or all proteins; dairy protein, egg protein, fish protein, fruit protein, grain protein, legume protein, lipoprotein, meat protein, nut protein, poultry protein, tofu protein, vegetable protein, complete protein, incomplete protein, or other amino acids; a selected type of fiber, a class of fiber, or all fiber; dietary fiber, insoluble fiber, soluble fiber, and cellulose; a specific sodium compound, a class of sodium compounds, and all sodium compounds; salt; a selected type of meat, a class of meats, and all meats; a selected type of vegetable, a class of vegetables, and all vegetables; a selected type of fruit, a class of fruits, and all fruits; a selected type of grain, a class of grains, and all grains; high-carbohydrate food, high-sugar food, high-fat food, fried food, high-cholesterol food, high-protein food, high-fiber food, and high-sodium food.

With respect to different quantities of food, there can be different metrics for measuring amounts of food, ingredients, and nutrients. Overall, amounts or quantities of food, ingredients, and nutrients can be measured in terms of volume, mass, or weight. Volume measures how much space the food occupies. Mass measures how much matter the food contains. Weight measures the pull of gravity on the food. The concepts of mass and weight are related, but not identical. Food, ingredient, or nutrient density can also be measured, sometimes as a step toward measuring food mass. In an example, volume can be expressed in metric units (such as cubic millimeters, cubic centimeters, or liters) or U.S. (historically English) units (such as cubic inches, teaspoons, tablespoons, cups, pints, quarts, gallons, or fluid ounces). Mass (and often weight in colloquial use) can be expressed in metric units (such as milligrams, grams, and kilograms) or U.S. (historically English) units (ounces or pounds). The density of specific ingredients or nutrients within food is sometimes measured in terms of the volume of specific ingredients or nutrients per total food volume or measured in terms of the mass of specific ingredients or nutrients per total food mass.

The optical sensor of a device can be a spectroscopic optical sensor. In an example, an optical sensor can be selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer. In an example, a device can include a light-based approach to food identification, such as spectroscopy. In an example, types of food, ingredients, and/or nutrients can be identified by the patterns of light that are reflected from, or absorbed by, the food at different wavelengths. In an example, an optical sensor can detect whether food reflects light at a different wavelength than the wavelength of light shone on food. In an example, an optical sensor can detect modulation of light reflected from, or absorbed by, a receptor when the receptor is exposed to food.

In an example, an optical sensor can analyze modulation of light wave parameters by the interaction of that light with a portion of food. In an example, an optical sensor can be a chromatographic sensor, spectrographic sensor, analytical chromatographic sensor, liquid chromatographic sensor, gas chromatographic sensor, optoelectronic sensor, photochemical sensor, or photocell. In an example, a device can comprise a sensor that is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

With respect to the one or more attachment mechanisms, an imaging member and an optical sensor can be attached to a person's body or clothing. In an example, an attachment mechanism can be selected from the group consisting of: band, strap, chain, hook and eye fabric, ring, adhesive, bracelet, buckle, button, clamp, clip, elastic band, eyewear, magnet, necklace, piercing, pin, string, suture, tensile member, wrist band, and zipper.

In an example, a device can be worn on a person in a manner like a clothing accessory or piece of jewelry selected from the group consisting of: wristwatch, wristphone, wristband, bracelet, cufflink, armband, armlet, and finger ring; necklace, neck chain, pendant, dog tags, locket, amulet, necklace phone, and medallion; eyewear, eyeglasses, spectacles, sunglasses, contact lens, goggles, monocle, and visor; clip, tie clip, pin, brooch, clothing button, and pin-type button; headband, hair pin, headphones, ear phones, hearing aid, earring; and dental appliance, palatal vault attachment, and nose ring. In an example, a device can be incorporated or integrated into an article of clothing or a clothing-related accessory. In various examples, a device can be incorporated or integrated into one of the following articles of clothing or clothing-related accessories: belt or belt buckle; neck tie; shirt or blouse; shoes or boots; underwear, underpants, briefs, undershirt, or bra; cap, hat, or hood; coat, jacket, or suit; dress or skirt; pants, jeans, or shorts; purse; socks; and sweat suit.

In an example, a device can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

In an example, the image-analyzing member can be a data control unit. In an example, the image-analyzing member can be a data control unit, data processing unit, data analysis component, Central Processing Unit (CPU), and/or microprocessor. In an example, an image-analyzing member can analyze pictures or images of food taken by the imaging member in order to estimate types and amounts of food, ingredients, nutrients, and/or calories. In an example, a device can comprise one or more components selected from the group consisting of: a data processing unit, data analysis component, Central Processing Unit (CPU), or microprocessor; a food-consumption monitoring component (motion sensor, electromagnetic sensor, optical sensor, and/or chemical sensor); a graphic display component (display screen and/or coherent light projection); a human-to-computer communication component (speech recognition, touch screen, keypad or buttons, and/or gesture recognition); a memory component (flash, RAM, or ROM); a power source and/or power-transducing component; a time keeping and display component; and a wireless data transmission and reception component.

In an example, a device can serve as the energy-input measuring component of an overall system for energy balance and weight management. In an example, a device can estimate the energy-input component of energy balance. In an example, information from a device can be combined with information from a separate caloric expenditure monitoring device that measures a person's caloric expenditure in order to comprise an overall system for energy balance, fitness, weight management, and health improvement. In an example, a device can be in wireless communication with a separate fitness monitoring device. In an example, the capability for monitoring food consumption can be combined with capability for monitoring caloric expenditure within a single device. In an example, a single device can be used to measure the types and amounts of food, ingredients, and/or nutrients that a person consumes as well as the types and durations of the calorie-expending activities in which the person engages.

### Using a Camera as an Imaging Member:

In an example, at least one imaging member can be a camera. In an example, a device, system, or method for measuring types of food, ingredients, or nutrients can include a camera, or other picture-taking device, that takes pictures of food. In an example, a device can comprise a camera with a field of vision which extends outwards from the camera aperture and downwards toward a reachable food source. In an example, a reachable food source can be food on a plate. In an example, a reachable food source can be encompassed by the field of vision. In an example, a camera can have an imaging vector that is generally perpendicular to the longitudinal bones of a person's upper arm. In an example, a camera can be positioned on a person's wrist at a location from which it takes pictures along an imaging vector that is directed generally downward from the imaging member toward a reachable food source as the person eats.

In an example, a camera can take pictures of the interaction between a person and food, including food apportionment, hand-to-mouth movements, and chewing movements. In an example, a device can be embodied in a device, system, and method for monitoring food consumption which comprises an imaging member, wherein this imaging member is used to take pictures of food that the person eats.

In an example, a device, system, or method for measuring food can include taking multiple pictures of food. In an example, taking pictures of food from at least two different angles can better segment a meal into different types of food, estimate the three-dimensional volume of each type of food, and control for lighting and shading differences. In an example, a camera or other imaging device can take pictures of food from multiple perspectives in order to create a virtual three-dimensional model of food in order to determine food volume. In an example, an imaging device can estimate the quantities of specific foods from pictures or images of those foods by volumetric analysis of food from multiple perspectives and/or by three-dimensional modeling of food from multiple perspectives.

In an example, a device can comprise at least two cameras or other imaging members. A first camera may be worn on a location on the human body from which it takes pictures along an imaging vector which points toward a person's mouth while the person eats. A second camera may be worn on a location on the human body from which it takes pictures along an imaging vector which points toward a reachable food source. In an example, a device can comprise two imaging members. A first imaging member can be worn on a person's wrist like a wrist watch. This first member can take pictures of the person's mouth. A second imaging member can be worn on a person's neck like a necklace. This second member takes pictures of the person's hand and a reachable food source.

Imaging Member that Faces Outward:

In an example, at least one imaging member can be configured to have a focal direction which points outward from the surface of a person's body or clothing. In an example, an imaging member can point outward and/or downward from the surface of a person's body or clothing in order to capture images of nearby food. In an example, an imaging member can point outward and/or downward from the surface of a person's body or clothing in order to capture images of the interaction between a person's hand and food. In an example, an imaging member can point outward and/or upward from the surface of a person's body or clothing in order to capture images of a person's mouth. In an example, an imaging member can point outward and/or upward from the surface of a person's body or clothing in order to capture images of the interaction between a person's mouth and food conveyed by person's hand. In an example, an imaging member can have a focal direction which is substantially perpendicular to the longitudinal bones of a person's upper arm. In an example, the focal direction of an imaging member can be configured along a vector which: points outward from a person's wrist or arm; and which is substantially perpendicular to the surface of a person's arm and/or the longitudinal bones of a person's arm.

In an example, a device can include a camera with a field of vision which extends outwards from the camera aperture and downwards toward a reachable food source. In an example, a reachable food source can be food on a plate. In an example, a reachable food source can be encompassed by the field of vision. In an example, a camera can be positioned on a person's wrist at a location from which it takes pictures along an imaging vector that is directed generally downward from the imaging member toward a reachable food source as the person eats. In an example, a camera can be positioned on a person's wrist at a location from which it takes pictures along an imaging vector that is directed generally upward from the imaging member toward the person's mouth as the person eats. In an example, a camera can have a field of vision which extends outwards from the camera aperture and upwards toward a person's mouth.

In an example, an imaging member can maintain a line of sight to one or both of a person's hands. In an example, an imaging member can scan for (and identify and maintain a line of sight to) a person's hand when one or more sensors indicate that the person is eating. In an example, an imaging member can scan for, acquire, and maintain a line of sight to a reachable food source when a sensor indicates that a person is probably eating. In an example, a device can monitor the location of a person's mouth. In an example, a device can monitor space around a person, especially space in the vicinity of the person's hand, to detect possible reachable food sources. In an example, a device can only monitor the location of a person's mouth, or scan for possible reachable food sources, when one or more sensors indicate that the person is probably eating.

In an example, a device can comprise at least two cameras or other imaging members. A first camera may be worn on a location on the human body from which it takes pictures along an imaging vector which points toward a person's mouth while the person eats. A second camera may be worn on a location on the human body from which it takes pictures along an imaging vector which points toward a reachable food source. In an example, a device may comprise two imaging members, or two cameras mounted on a single member, which are generally perpendicular to the longitudinal bones of the upper arm. In an example, one of these imaging members can have an imaging vector that points toward a food source at different times. In an example, another one of these imaging members may have an imaging vector that points toward the person's mouth at different times. In an example, these different imaging vectors may occur simultaneously as a body moves and/or food travels. In another example, these different imaging vectors may occur sequentially as a body moves and/or food travels. This device and method can provide images from multiple imaging vectors, such that these images from multiple perspectives are automatically and collectively analyzed to identify the types and quantities of food consumed by a person.

In an example, a camera that is used for identifying food can have a variable focal length. In an example, the imaging vector and/or focal distance of a camera can be actively and automatically adjusted to focus on: the person's hands, space surrounding the person's hands, a reachable food source, a food package, a menu, the person's mouth, and the person's face. In an example, in the interest of privacy, the focal length of a camera can be automatically adjusted in order to focus on food and not other people.

### Spectroscopic Optical Sensor:

In an example, the optical sensor can be a spectroscopic optical sensor. In an example, an optical sensor can be a spectroscopic optical sensor that collects data concerning the spectrum of light that is transmitted through and/or reflected from nearby food. In an example, an optical sensor can be selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer. In an example, an optical sensor can analyze modulation of light wave parameters by the interaction of that light with a portion of food. In an example, an optical sensor can detect modulation of light reflected from, or absorbed by, a receptor when the receptor is exposed to food.

In an example, a device can comprise a sensor selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor. In an example, an optical sensor can be a chromatographic sensor, spectrographic sensor, analytical chromatographic sensor, liquid chromatographic sensor, gas chromatographic sensor, optoelectronic sensor, photochemical sensor, and photocell.

In an example, a device can identify a type of food by optically analyzing food. In an example, a device can identify types and amounts of food by recording the effects of light that is interacted with food. In an example, a device can identify the types and amounts of food consumed via spectroscopy. In an example, types of food, ingredients, and/or nutrients can be identified by the patterns of light that are reflected from, or absorbed by, food at different wavelengths. In an example, a light-based sensor can detect food consumption or can identify consumption of a specific food, ingredient, or nutrient based on the reflection of light from food or the absorption of light by food at different wavelengths. In an example, an optical sensor can detect whether food reflects light at a different wavelength than the wavelength of light shone on food. In an example, a light-based sensor can identify consumption of a selected type of food, ingredient, or nutrient with a spectral analysis sensor. In an example, a device can comprise a light-based approach to food identification such as spectroscopy. In an example, an optical sensor can emit and/or detect white light, infrared light, or ultraviolet light.

In an example, a device can comprise a sensor which collects information concerning the wavelength spectra of light reflected from, or absorbed by, food. In an example, a device can comprise a sensor that identifies types of food, ingredients, or nutrients by detecting light reflection spectra, light absorption spectra, or light emission spectra. In an example, a spectral measurement sensor can be a spectroscopy sensor or a spectrometry sensor. In an example, a spectral measurement sensor can be a white light spectroscopy sensor, an infrared spectroscopy sensor, a near-infrared spectroscopy sensor, an ultraviolet spectroscopy sensor, an ion mobility spectroscopic sensor, a mass spectrometry sensor, a backscattering spectrometry sensor, or a spectrophotometer. In an example, light at different wavelengths can be absorbed by, or reflected off, food and the results can be analyzed in spectral analysis.

In an example, a device can analyze the chemical composition of food by measuring the effects of the interaction between food and light energy. In an example, this interaction can comprise the degree of reflection or absorption of light by food at different light wavelengths. In an example, this interaction can include spectroscopic analysis. In an example, a device can collect data that is used to analyze the chemical composition of food by measuring the absorption of light, sound, or electromagnetic energy by food that is in proximity to a person. In an example, a device can collect data that is used to analyze the chemical composition of food by measuring the reflection of different wavelengths of light, sound, or electromagnetic energy by food that is in proximity to a person. In an example, a device can comprise a sensor that identifies a selected type of food, ingredient, or nutrient by detecting light reflection spectra, light absorption spectra, or light emission spectra.

### Outward-Facing Optical Sensor:

In an example, an optical sensor can be configured to have a sensing direction which points outward from the surface of a person's body or clothing. In an example, an optical sensor can point outward and/or downward from the surface of a person's body or clothing in order to capture light transmitted through and/or reflected from nearby food. In an example, an optical sensor can have a sensing direction which is substantially perpendicular to the longitudinal bones of a person's upper arm. In an example, the sensing direction of an optical sensor can be configured along a vector which: points outward from a person's wrist or arm; and which is substantially perpendicular to the surface of a person's arm and/or the longitudinal bones of a person's arm.

In an example, a device can collect data that is used to analyze the chemical composition of food by measuring the absorption of light, sound, or electromagnetic energy by food that is in proximity to the person whose consumption is being monitored. In an example, a device can collect data that is used to analyze the chemical composition of food by measuring the reflection of different wavelengths of light, sound, or electromagnetic energy by food that is in proximity to the person whose consumption is being monitored. In an example, a device can comprise a sensor which collects information concerning the wavelength spectra of light reflected from, or absorbed by, food.

### Attachment Mechanisms:

In an example, one or more attachment mechanisms can be selected from the group consisting of: arm band, bracelet, brooch, collar, cuff link, dog tags, ear ring, ear-mounted bluetooth device, eyeglasses, finger ring, headband, hearing aid, necklace, pendant, wearable mouth microphone, wrist band, and wrist watch. In an example, one or more attachment mechanisms can be selected from the group consisting of: wrist watch, wrist band, bracelet, arm band, necklace, pendant, brooch, collar, eyeglasses, ear ring, headband, or ear-mounted bluetooth device. In an example, one or more attachment mechanisms can be selected from the group consisting of: wrist watch, bracelet, finger ring, necklace, or ear ring. In an example, one or more attachment mechanisms can be selected from the group consisting of: necklace; pendant, dog tags; brooch; cuff link; ear ring; eyeglasses; wearable mouth microphone; and hearing aid.

In an example, one or more attachment mechanisms can be worn like a clothing accessory or piece of jewelry selected from the group consisting of: wristwatch, wristphone, wristband, bracelet, cufflink, armband, armlet, and finger ring; necklace, neck chain, pendant, dog tags, locket, amulet, necklace phone, and medallion; eyewear, eyeglasses, spectacles, sunglasses, contact lens, goggles, monocle, and visor; clip, tie clip, pin, brooch, clothing button, and pin-type button; headband, hair pin, headphones, ear phones, hearing aid, earring; and dental appliance, palatal vault attachment, and nose ring.

In an example, one or more attachment mechanisms can be worn like a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

In an example, a device or system for measuring a person's consumption of types of food, ingredients, and/or nutrients can take pictures of food using a device selected from the group consisting of: smart watch, smart bracelet, fitness watch, fitness bracelet, watch phone, bracelet phone, wrist band, or other wrist-worn device; arm bracelet; and smart ring or finger ring. In an example, a wearable sensor can be part of an electronically-functional wrist band or smart watch.

In an example, a device or system can be attached to a person's body or clothing. In an example, an attachment mechanism can be selected from the group consisting of: band, strap, chain, hook and eye fabric, ring, adhesive, bracelet, buckle, button, clamp, clip, elastic band, eyewear, magnet, necklace, piercing, pin, string, suture, tensile member, wrist band, and zipper. In an example, a device or system can be attached to a person or to a person's clothing by a means selected from the group consisting of: strap, clip, clamp, snap, pin, hook and eye fastener, magnet, and adhesive.

In an example, a device can be worn on, or attached to, a person's body. In an example, a device can be worn on, or attached to, a person's clothing. In an example, a device can be incorporated into the creation of a specific article of clothing. In an example, a device can be integrated into a specific article of clothing by a means selected from the group consisting of: adhesive, band, buckle, button, clip, elastic band, hook and eye fabric, magnet, pin, pocket, pouch, sewing, strap, tensile member, and zipper. In an example, a device for measuring a person's food consumption can be incorporated or integrated into an article of clothing or a clothing-related accessory.

In an example, a device or system can be worn on, or attached to, one or more parts of a person's body that are selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg. In various examples, a device can be incorporated or integrated into one of the following articles of clothing or clothing-related accessories: belt or belt buckle; neck tie; shirt or blouse; shoes or boots; underwear, underpants, briefs, undershirt, or bra; cap, hat, or hood; coat, jacket, or suit; dress or skirt; pants, jeans, or shorts; purse; socks; and sweat suit.

In an example, a device can have an unobtrusive, or even attractive, design like a piece of jewelry. In an example, a device for measuring a person's consumption of at least one selected type of food, ingredient, or nutrient can be worn in a manner similar to a piece of jewelry or accessory. In an example, a wearable sensor can be part of an electronically-functional adhesive patch that can be worn on a person's skin.

### Image-Analyzing Member and Methods of Image Analysis:

In an example, the image-analyzing member can be a data control unit. In an example, the image-analyzing member can be selected from the group consisting of: a data control unit, a data processing unit, a data analysis component, a Central Processing Unit (CPU), and a microprocessor. In an example, an image-analyzing member can analyze pictures or images of food taken by an imaging member in order to estimate types and amounts of foods, ingredients, nutrients, and/or calories. In an example, a device can comprise a data analysis component, wherein this component analyzes pictures of food taken by an imaging member to estimate types and amounts of foods, ingredients, nutrients, and/or calories.

In an example, an image-analyzing member and/or a data control unit can comprise one or more components selected from the group consisting of: a data processing unit, data analysis component, Central Processing Unit (CPU), or microprocessor; a food-consumption monitoring component (motion sensor, electromagnetic sensor, optical sensor, and/or chemical sensor); a graphic display component (display screen and/or coherent light projection); a human-to-computer communication component (speech recognition, touch screen, keypad or buttons, and/or gesture recognition); a memory component (flash, RAM, or ROM); a power source and/or power-transducing component; a time keeping and display component; and a wireless data transmission and reception component.

In an example, a image-analyzing member and/or a data control unit can comprise one or more components selected from the group consisting of: a food-consumption monitor or food-identifying sensor; a central processing unit (CPU) such as a microprocessor; a database of different types of food and food attributes; a memory to store, record, and retrieve data such as the cumulative amount consumed for at least one selected type of food, ingredient, or nutrient; a communications member to transmit data to from external sources and to receive data from external sources; a power source such as a battery or power transducer; a human-to-computer interface such as a touch screen, keypad, or voice recognition interface; and a computer-to-human interface such as a display screen or voice-producing interface.

In an example, a device can further comprise one or more components selected from the group consisting of: a data processing unit, data analysis component, Central Processing Unit (CPU), or microprocessor; a food-consumption monitoring component (motion sensor, electromagnetic sensor, optical sensor, and/or chemical sensor); a graphic display component (display screen and/or coherent light projection); a human-to-computer communication component (speech recognition, touch screen, keypad or buttons, and/or gesture recognition); a memory component (flash, RAM, or ROM); a power source and/or power-transducing component; a time keeping and display component; and a wireless data transmission and reception component.

In an example, a device can further comprise one or more components selected from the group consisting of: a food-consumption monitor or food-identifying sensor; a central processing unit (CPU) such as a microprocessor; a database of different types of food and food attributes; a memory to store, record, and retrieve data such as the cumulative amount consumed for at least one selected type of food, ingredient, or nutrient; a communications member to transmit data to from external sources and to receive data from external sources; a power source such as a battery or power transducer; a human-to-computer interface such as a touch screen, keypad, or voice recognition interface; and a computer-to-human interface such as a display screen or voice-producing interface.

In an example, an image-analyzing member and/or a data control unit can be part of a wearable device or can be the wearable component of a system. In an example, data concerning food consumption that is collected by a wearable device can be analyzed by an image-analyzing member and/or a data control unit within the wearable device in order to identify the types and amounts of foods, ingredients, or nutrients that a person consumes. In another example, an image-analyzing member and/or a data control unit can be in a remote location and in wireless communication to receive data from a wearable device or the wearable component of a system.

In an example, automated identification of types of food based on images and/or automated association of selected types of ingredients or nutrients with that food can occur within a wearable device. In an example, data collected by a wearable device can be transmitted to an external device wherein automated identification occurs and the results can then be transmitted back to the wearable device. In an example, food image information can be transmitted from a wearable device to a remote location wherein automatic food identification occurs and the results can be transmitted back to the wearable device. In another example, data concerning food consumption that is collected by a wearable device can be transmitted to an external device or system for analysis at a remote location. In an example, pictures of food can be transmitted to an external device or system for food identification at a remote location. In an example, chemical analysis results can be transmitted to an external device or system for food identification at a remote location. In an example, the results of analysis at a remote location can be transmitted back to a wearable device.

In an example, a food-consumption monitoring and nutrient identifying system can include a component that is selected from the group consisting of: smart phone, mobile phone, cell phone, or application of such a phone; electronic tablet, other flat-surface mobile electronic device, Personal Digital Assistant (PDA), or laptop; digital camera; and smart eyewear, electronically-functional eyewear, or augmented reality eyewear. In an example, such a component can be in wireless communication with another component of such a system. In an example, a device for measuring food consumption can be in wireless communication with an external device selected from the group consisting of: internet portal; smart phone, mobile phone, cell phone, or application of such a phone; electronic tablet, other flat-surface mobile electronic device, Personal Digital Assistant (PDA), remote control unit, or laptop; smart eyewear, electronically-functional eyewear, or augmented reality eyewear; electronic store display, electronic restaurant menu, or vending machine; and desktop computer, television, or mainframe computer. In an example, a device, method, or system for detecting food consumption or measuring consumption of a selected type of food, ingredient, or nutrient can include integration with a general-purpose mobile device that is used to collects data concerning food consumption. In an example, a component of such a system can be a general purpose device, of which collecting data for food identification is only one among many functions that it performs.

In an example, an imaging member and an optical sensor can be in wireless communication with each other or other devices. In an example, a device or system for measuring a person's consumption of types of food, ingredients, or nutrients can include one or more communications components for wireless transmission and reception of data. In an example, multiple communications components can enable wireless communication (including data exchange) between separate components of such a device and system. In an example, a communications component can enable wireless communication with an external device or system. In various examples, the means of this wireless communication can be selected from the group consisting of: radio transmission, Bluetooth transmission, Wi-Fi, and infrared energy.

In an example, food can be identified directly by wireless information received from a food display, RFID tag, electronically-functional restaurant menu, or vending machine. In an example, food or its nutritional composition can be identified directly by wireless transmission of information from a food display, menu, food vending machine, food dispenser, or other point of food selection or sale and a device that is worn, held, or otherwise transported with a person. In various examples, a device can receive food-identifying information from a source selected from the group consisting of: electromagnetic transmissions from a food display or RFID food tag in a grocery store, electromagnetic transmissions from a physical menu or virtual user interface at a restaurant, and electromagnetic transmissions from a vending machine. With respect to meals ordered at restaurants, some restaurants (especially fast-food restaurants) have standardized menu items with standardized food ingredients. In such cases, identification of types and amounts of food, ingredients, or nutrients can be conveyed at the point of ordering (via an electronically-functional menu) or purchase (via purchase transaction).

In an example, a device and system for measuring a person's consumption of at least one selected type of food, ingredient, or nutrient can identify and track food consumption at the point of selection or point of sale. In an example, a device or system for monitoring food consumption or consumption of selected types of food, ingredients, or nutrients can approximate such measurements by tracking a person's food selections and purchases at a grocery store, at a restaurant, or via a vending machine. In an example, such tracking can be done with specific methods of payment, such as a credit card or bank account. In an example, such tracking can be done with electronically-functional food identification means such as bar codes, RFID tags, or electronically-functional restaurant menus. Electronic communication for food identification can also occur between a food-consumption monitoring device and a vending machine.

In various examples, food may be identified by pattern recognition of food itself, by recognition of words on food packaging or containers, by recognition of food brand images and logos, or by recognition of product identification codes (such as “bar codes”). In an example, a device for measuring a person's consumption of at least one selected type of food, ingredient, or nutrient can identify food using information from a food's packaging or container. In an example, food can be identified directly by automated recognition of information on food packaging, such as a logo, label, or barcode. In various examples, information on a food's packaging or container that is used to identify the type and/or amount of food can be selected from the group consisting of: bar code, food logo, food trademark design, nutritional label, optical text recognition, and UPC code. Food can be identified by scanning a barcode or other machine-readable code on the food's packaging (such as a Universal Product Code or European Article Number), on a menu, on a store display sign, or otherwise in proximity to food at the point of food selection, sale, or consumption. In an example, the type of food (and/or specific ingredients or nutrients within the food) can be identified by machine-recognition of a food label, nutritional label, or logo on food packaging, menu, or display sign.

In an example, a device for measuring types of food, ingredients, or nutrients can identify the types and amounts of food in an automated manner based on analyzing pictures or images of that food. In an example, identification of the types and quantities of foods, ingredients, or nutrients from pictures or images of food can be a combination of, or interaction between, automated food identification methods and human-based food identification methods. In an example, a device can identify and track the selected types and amounts of foods, ingredients, or nutrients in an entirely automatic manner. In an example, such identification can occur in a partially automatic manner in which there is interaction between automated and human identification methods.

In an example, methods for automatic identification of food types and amounts from food pictures can include: color analysis, image pattern recognition, image segmentation, texture analysis, three-dimensional modeling based on pictures from multiple perspectives, and volumetric analysis based on a fiducial marker or other object of known size. In an example, a device can use one or more methods to analyze pictures of images of food wherein these methods are selected from the group consisting of: 3D modeling, bar code recognition or identification, changes in food at a reachable food source, face recognition or identification, food recognition or identification, gesture recognition or identification, human motion recognition or identification, logo recognition or identification, pattern recognition or identification, number of cycles of food moving along a food consumption pathway, and word recognition or identification. In an example, images of a person's mouth and a reachable food source may be taken from at least two different perspectives in order to enable the creation of three-dimensional models of food.

In example, a device can comprise one or more image-analyzing members that analyze one or more factors selected from the group consisting of: number and type of reachable food sources; changes in the volume of food observed at a reachable food source; number and size of chewing movements; number and size of swallowing movements; number of times that pieces (or portions) of food travel along the food consumption pathway; and size of pieces (or portions) of food traveling along the food consumption pathway. In various examples, one or more of these factors may be used to analyze images to estimate the types and quantities of food consumed by a person. In example, a device can comprise one or more image-analyzing members that analyze one or more factors selected from the group consisting of: one or more factors selected from the group consisting of: number of reachable food sources; types of reachable food sources; changes in the volume of food at a reachable food source; number of times that the person brings food to their mouth; sizes of portions of food that the person brings to their mouth; number of chewing movements; frequency or speed of chewing movements; and number of swallowing movements.

In an example, a device can use one or more methods to analyze pictures of images of food wherein these methods are selected from the group consisting of: image attribute adjustment or normalization; inter-food boundary determination and food portion segmentation; image pattern recognition and comparison with images in a food database to identify food type; comparison of a vector of food characteristics with a database of such characteristics for different types of food; scale determination based on a fiducial marker and/or three-dimensional modeling to estimate food quantity; and association of selected types and amounts of ingredients or nutrients with selected types and amounts of food portions based on a food database that links common types and amounts of foods with common types and amounts of ingredients or nutrients.

In an example, a device can use one or more methods to analyze pictures of images of food wherein these methods are selected from the group consisting of: analysis of variance (ANOVA), Chi-squared analysis, cluster analysis, color and texture analysis, comparison of a vector of food parameters with a food database containing such parameters, comparison with food images with food images in a food database, energy balance tracking, factor analysis, food portion segmentation, Fourier transformation and/or fast Fourier transform (FFT), image attribute adjustment or normalization, image pattern recognition, image segmentation, inter-food boundary determination, linear discriminant analysis, linear regression, logistic regression, multivariate linear regression, neural network and machine learning, non-linear programming, pattern recognition, principal components analysis, probit analysis, scale determination using a physical or virtual fiducial marker, survival analysis, three-dimensional modeling, time series analysis, volumetric analysis based on a fiducial marker or other object of known size, and volumetric modeling.

In an example, a device can take multiple still pictures or moving video pictures of food. In an example, a device can take multiple pictures of food from different angles in order to perform three-dimensional analysis or modeling of the food to better determine the volume of food. In an example, a device can take multiple pictures of food from different angles in order to better control for differences in lighting and portions of food that are obscured from some perspectives. In an example, a device can take multiple pictures of food from different angles in order to perform three-dimensional modeling or volumetric analysis to determine the three-dimensional volume of food in the picture. In an example, volume estimation can include obtaining video images of food or multiple still pictures of food in order to obtain pictures of food from multiple perspectives. In an example, pictures of food from multiple perspectives can be used to create three-dimensional or volumetric models of that food in order to estimate food volume. In an example, multiple pictures of food from different angles can enable three-dimensional modeling of food volume.

In an example, a device can comprise two or more imaging members wherein a first imaging member is pointed toward a person's mouth most of the time, as the person moves their arm to move food, and wherein a second imaging member is pointed toward a reachable food source most of the time, as the person moves their arm to move food. In an example, a device can comprise one or more imaging members wherein: a first imaging member points toward a person's mouth at least once as the person brings a piece (or portion) of food to their mouth from a reachable food source; and a second imaging member points toward the reachable food source at least once as the person brings a piece (or portion) of food to their mouth from the reachable food source.

In an example, a device can further comprise a locally or remotely housed food database. In an example, a food database can be used to identify food types and quantify food amounts. In an example, a device can collect food images that are automatically associated with images of food in a food database for food identification. In an example, analysis of images can occur in real time, as a person is consuming food. In an example, analysis of images by this device and method can occur after a person has consumed food.

In an example, a food database can include one or more elements selected from the group consisting of: food name, food picture (individually or in combinations with other foods), food color, food shape, food texture, food type, food packaging bar code or nutritional label, food packaging or logo pattern, common geographic or intra-building locations for serving or consumption, common or standardized ingredients (per serving, per volume, or per weight), common or standardized number of calories (per serving, per volume, or per weight), common or standardized nutrients (per serving, per volume, or per weight), common or standardized size (per serving), common times or special events for serving or consumption, and commonly associated or jointly-served foods.

The concepts of food identification, ingredient identification, and nutrient identification are closely related. Various embodiments of a device can identify specific ingredients or nutrients indirectly (through food identification and use of a database) or directly (through the use of nutrient-specific sensors such as a spectroscopic optical sensor). In an example, a food database can be used to link common types and quantities of ingredients or nutrients with common types and quantities of food. In an example, types and quantities of ingredients and/or nutrients can be estimated indirectly using a database that links common types and amounts of food with common types and amounts of ingredients or nutrients. In an example, a device can directly identify types and quantities of ingredients and/or nutrients. The latter does not rely on estimates from a database, but does require ingredient-specific or nutrient-specific sensors (such as a spectroscopic optical sensor).

In an example, the amount of a specific ingredient or nutrient within (a portion of) food can be measured directly by a sensing mechanism. In an example, the amount of a specific ingredient or nutrient within (a portion of) food can be estimated indirectly by measuring the amount of food and then linking this amount of food to amounts of ingredients or nutrients using a database that links specific foods with standard amounts of ingredients or nutrients. In an example, specific ingredients or nutrients that are associated with selected types of food can be estimated based on a database linking foods to ingredients and nutrients.

In an example, a device, method, or system for measuring a person's consumption of at least one selected type of food, ingredient, or nutrient can identify and track a person's food consumption at the point of consumption. In an example, such a device, method, or system can include a database of different types of food. In an example, such a device, method, or system can be in wireless communication with an externally-located database of different types of food. In an example, such a database of different types of food and their associated attributes can be used to help identify selected types of food, ingredients, or nutrients. In an example, a database of attributes for different types of food can be used to associate types and amounts of specific ingredients, nutrients, and/or calories with selected types and amounts of food.

In an example, a food database can be used to identify the amount of calories that are associated with an indentified type and amount of food. In an example, a food database can be used to identify the type and amount of at least one selected type of food that a person consumes. In an example, a food database can be used to identify the type and amount of at least one selected type of ingredient that is associated with an identified type and amount of food. In an example, a food database can be used to identify the type and amount of at least one selected type of nutrient that is associated with an identified type and amount of food. In an example, an ingredient or nutrient can be associated with a type of food on a per-portion, per-volume, or per-weight basis.

In an example, for some foods with standardized sizes (such as foods that are manufactured in standard sizes at high volume), food weight can be estimated as part of food identification. In an example, information concerning the weight of food consumed can be linked to nutrient quantities in a computer database in order to estimate cumulative consumption of selected types of nutrients. In an example, a food database can also include average amounts of specific ingredients and/or nutrients associated with specific types and amounts of foods for measurement of at least one selected type of ingredient or nutrient. In an example, a food database can be used to identify the type and amount of at least one selected type of ingredient that is associated with an identified type and amount of food.

In an example, attributes of food in an image can be represented by a multi-dimensional food attribute vector. In an example, this food attribute vector can be statistically compared to the attribute vector of known foods in order to automate food identification. In an example, multivariate analysis can be done to identify the most likely identification category for a particular portion of food in an image. In an example, automatic identification of food amounts and types can include extracting a vector of food parameters (such as color, texture, shape, and size) from a food picture and comparing this vector with vectors of these parameters in a food database. In various examples, a multi-dimensional food attribute vector can include attributes selected from the group consisting of: food color; food texture; food shape; food size or scale; geographic location of selection, purchase, or consumption; timing of day, week, or special event; common food combinations or pairings; image brightness, resolution, or lighting direction; infrared light reflection; spectroscopic analysis; and person-specific historical eating patterns.

In an example, images of food can be automatically analyzed in order to identify types and quantities of food. In an example, pictures of food taken by a camera or other picture-taking device can be automatically analyzed to estimate the types and amounts of food, ingredients, or nutrients. In an example, an initial stage of an image analysis system can comprise adjusting, normalizing, or standardizing image elements for better food segmentation, identification, and volume estimation. In an example, a device can identify specific foods from pictures or images by image segmentation, color analysis, texture analysis, and pattern recognition.

In an example, there can be a preliminary stage of processing or analysis of food pictures wherein image elements and/or attributes are adjusted, normalized, or standardized. In an example, a food picture can be adjusted, normalized, or standardized before it is compared with food pictures in a food database. This can improve segmentation of a meal into different types of food, identification of foods, and estimation of food volume or mass.

In an example, food lighting or shading can be adjusted, normalized, or standardized before comparison with pictures in a food database. In an example, food size or scale can be adjusted, normalized, or standardized before comparison with pictures in a food database. In an example, food texture can be adjusted, normalized, or standardized before comparison with pictures in a food database. In various examples a preliminary stage of food picture processing and/or analysis can include adjustment, normalization, or standardization based on one or more factors selected from the group consisting of: adjacent foods, context, food color, food shape, food size, food texture, food texture, geographic location, image brightness, image resolution, light angle, place setting context, scale, and temperature (infrared).

In an example, analysis of food images can include the step of automatically segmenting regions of a food image into different types or portions of food. In an example, a picture of a meal as a whole can be automatically segmented into portions of different types of food for comparison with different types of food in a food database. In an example, a device can automatically identify boundaries between different types of food in an image that contains multiple types or portions of food. In an example, the creation of boundaries between different types of food and/or segmentation of a meal into different food types can include edge detection, shading analysis, texture analysis, and three-dimensional modeling. In an example, this process can also be informed by common patterns of jointly-served foods and common boundary characteristics of such jointly-served foods.

In an example, an imaging device can take pictures of food at different times, such as before and after an eating event, in order to better determine how much food the person actually ate (as compared to the amount of food served or nearby). In an example, pictures of food at different times (such as before and after a meal) can enable estimation of the amount of proximal food that is actually consumed vs. just being served in proximity to the person. In an example, changes in the volume of food in sequential pictures before and after consumption can be compared to the cumulative volume of food conveyed to a person's mouth to determine a more accurate estimate of food volume consumed.

In an example, a method for measuring a person's consumption of types of food, ingredients, or nutrients can include monitoring changes in the volume or weight of food at a reachable location near the person. In an example, pictures of food can be taken at multiple times before, during, and after food consumption in order to better estimate the amount of food that the person actually consumes, which can differ from the amount of food served to the person or the amount of food left over after the person eats. In an example, estimates of the amount of food that the person actually consumes can be made by digital image subtraction and/or 3D modeling. In an example, changes in the volume or weight of nearby food can be correlated with hand motions in order to estimate the amount of food that a person actually eats. In an example, a device can track the cumulative number of hand-to-mouth motions, number of chewing motions, or number of swallowing motions.

In an example, a device can collect data that enables tracking the cumulative amount of foods, ingredients, and/or nutrients which a person consumes during a period of time (such as an hour, day, week, or month) or during a particular eating event. In an example, the time boundaries of a particular eating event can be defined by a maximum time between chews or mouthfuls during a meal and/or a minimum time between chews or mouthfuls between meals. In an example, the time boundaries of a particular eating event can be defined by Fourier Transformation analysis of the variable frequencies of chewing, swallowing, or biting during meals vs. between meals.

In an example, a standard or target cumulative amount of food, ingredient, or nutrient consumption can be selected from the group consisting of: daily recommended minimum amount; daily recommended maximum amount or allowance; weekly recommended minimum amount; weekly recommended maximum amount or allowance; target amount to achieve a health goal; and maximum amount or allowance per meal. In an example, a standard amount can be a Reference Daily Intake (RDI) value or a Daily Reference Value.

In an example, analysis of cumulative food consumption can include comparison of food consumption parameters between a specific person and a reference population. In an example, data analysis can include analysis of a person's food consumption patterns over time. In an example, such analysis can track the cumulative amount of at least one selected type of food, ingredient, or nutrient that a person consumes during a selected period of time. In an example, an amount of a selected type of food, ingredient, or nutrient consumed can be expressed as an absolute amount. In an example, an amount of a selected type of food, ingredient, or nutrient consumed can be expressed as a percentage of a standard amount.

In an example, a target amount of cumulative food, ingredient, or nutrient consumption can be based on one or more factors selected from the group consisting of: the selected type of selected food, ingredient, or nutrient; amount of this type recommended by a health care professional or governmental agency; specificity or breadth of the selected nutrient type; the person's age, gender, and/or weight; the person's diagnosed health conditions; the person's exercise patterns and/or caloric expenditure; the person's physical location; the person's health goals and progress thus far toward achieving them; one or more general health status indicators; magnitude and/or certainty of the effects of past consumption of the selected nutrient on the person's health; the amount and/or duration of the person's consumption of healthy food or nutrients; changes in the person's weight; time of day; day of the week; occurrence of a holiday or other occasion involving special meals; dietary plan created for the person by a health care provider; input from a social network and/or behavioral support group; input from a virtual health coach; health insurance copay and/or health insurance premium; financial payments, constraints, and/or incentives; cost of food; speed or pace of nutrient consumption; and accuracy of a sensor in detecting the selected nutrient.

In an example, a device can include a computer-to-human interface. In an example, a computer-to-human interface can provide information and/or feedback to a person wearing a device, wherein the person's food consumption and/or nutritional intake is changed if the person volitionally changes their food consumption behavior based on this information and/or feedback. In an example, a device can provide information and/or feedback concerning food consumption to a person. In an example, a computer-to-human interface can communicate information about the types and amounts of food that a person has consumed, should consume, or should not consume. In an example, a computer-to-human interface can provide feedback to a person concerning their eating habits and the effects of those eating habits.

In an example, a device can provide information and/or feedback to a person that is selected from the group consisting of: feedback concerning food consumption (such as types and amounts of foods, ingredients, and nutrients consumed, calories consumed, calories expended, and net energy balance during a period of time); information about good or bad ingredients in nearby food; information concerning financial incentives or penalties associated with acts of food consumption and achievement of health-related goals; information concerning progress toward meeting a weight, energy-balance, and/or other health-related goal; information concerning the calories or nutritional components of specific food items; and number of calories consumed per eating event or time period.

Information from a device can be combined with a computer-to-human interface that provides feedback to encourage the person to eat healthy foods and to limit excess consumption of unhealthy foods. In order to be really useful for achieving good nutrition and health goals, a device, system, and method for measuring food consumption should differentiate between a person's consumption of healthy foods versus unhealthy foods. A device, system, or method can monitor a person's eating habits to encourage consumption of healthy foods and to discourage excess consumption of unhealthy foods.

In an example, a device can provide information and/or feedback concerning the types and quantities of nearby food. In an example, a device can provide information and/or feedback on the types and quantities of ingredients or nutrients in nearby food. In an example, a device can provide a person with information and/or feedback on the types and quantities of food that the person is consuming. In an example, a device can provide a person with information and/or feedback on the types and quantities ingredients or nutrients in food that the person is consuming. In an example, a device can provide a person with information and/or feedback on their cumulative consumption types of food, ingredients, or nutrients.

In an example, a device can track the cumulative amount of a food, ingredient, or nutrient consumed by the person and provide feedback to the person based on the person's cumulative consumption relative to a target amount. In an example, a device can provide negative feedback when a person exceeds a target amount of cumulative consumption. In an example, a device and system can sound an alarm or provide other real-time feedback to a person when the consumed amount of a selected type of food, ingredient, or nutrient exceeds an allowable amount (in total, per meal, or per unit of time).

Information from a food-consumption monitoring device that measures a person's consumption of at least one selected type of food, ingredient, and/or nutrient can also be combined with a computer-to-human interface that provides feedback to encourage the person to eat healthy foods and to limit excess consumption of unhealthy foods. In an example, capability for monitoring food consumption can be combined with capability for providing behavior-modifying feedback within a single device. In an example, a single device can be used to measure the selected types and amounts of foods, ingredients, and/or nutrients that a person consumes and to provide visual, auditory, tactile, or other feedback to encourage the person to eat in a healthier manner.

In an example, a device can provide information and/or feedback to a person that is selected from the group consisting of: augmented reality feedback (such as virtual visual elements superimposed on foods within a person's field of vision); changes in a picture or image of a person reflecting the likely effects of a continued pattern of food consumption; display of a person's progress toward achieving energy balance, weight management, dietary, or other health-related goals; graphical display of foods, ingredients, or nutrients consumed relative to standard amounts (such as embodied in pie charts, bar charts, percentages, color spectrums, icons, emoticons, animations, and morphed images); graphical representations of food items; graphical representations of the effects of eating particular foods; information on a computer display screen (such as a graphical user interface); lights, pictures, images, or other optical feedback; touch screen display; and visual feedback through electronically-functional eyewear. In an example, an amount of a selected type of food, ingredient, or nutrient consumed can be displayed as a portion of a standard amount such as in a bar chart, pie chart, thermometer graphic, or battery graphic.

In an example, a computer-to-human interface of a device can be used to not just provide information concerning eating behavior, but also to actively change eating behavior, nutritional intake, and/or nutritional absorption. In an example, a device can be in wireless communication with a separate feedback device that modifies the person's nutritional intake. In an example, a device can deliver neural stimulation (or be in wireless communication with a separate device which delivers neural stimulation) in order to modify a person's nutritional intake. In an example, a device can create a phantom taste or smell (or be in wireless communication with a separate device which creates a phantom taste or smell) in order to modify a person's nutritional intake. In an example, a device can exert pressure (or be in wireless communication with a separate device which exerts pressure) in order to modify a person's nutritional intake.

In an example, a device can include a computer-to-human interface that is selected from the group consisting of: auditory feedback (such as a voice message, alarm, buzzer, ring tone, or song); feedback via computer-generated speech; mild external electric charge or neural stimulation; periodic feedback at a selected time of the day or week; phantom taste or smell; phone call; pre-recorded audio or video message by the person from an earlier time; television-based messages; and tactile, vibratory, or pressure-based feedback. In another example, a computer-to-human interface can comprise one or more mechanisms which actively change a person's food consumption and/or nutritional intake from consumed food.

In an example, a device can engage other people as well as the person wearing the device. In an example, a device can provide feedback selected from the group consisting of: advice concerning consumption of specific foods or suggested food alternatives (such as advice from a dietician, nutritionist, nurse, physician, health coach, other health care professional, virtual agent, or health plan); electronic verbal or written feedback (such as phone calls, electronic verbal messages, or electronic text messages); live communication from a health care professional; questions to the person that are directed toward better measurement or modification of food consumption; real-time advice concerning whether to eat specific foods and suggestions for alternatives if foods are not healthy; social feedback (such as encouragement or admonitions from friends and/or a social network); suggestions for meal planning and food consumption for an upcoming day; and suggestions for physical activity and caloric expenditure to achieve desired energy balance outcomes.

In an example, a device can also include a human-to-computer interface for communication from a human to a computer. This human-to-computer interface can be selected from the group consisting of: speech recognition or voice recognition interface; touch screen or touch pad; physical keypad/keyboard, virtual keypad or keyboard, control buttons, or knobs; gesture recognition interface; motion recognition clothing; eye movement detector, smart eyewear, and/or electronically-functional eyewear; head movement tracker; conventional flat-surface mouse, 3D blob mouse, track ball, or electronic stylus; graphical user interface, drop down menu, pop-up menu, or search box; and neural interface or EMG sensor.

In an example, a device can further comprise a power source that is selected from the group consisting of: power from a power source that is internal to a device during regular operation (such as an internal battery, capacitor, energy-storing microchip, or wound coil or spring); power that is obtained, harvested, or transduced from a power source other than the person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from the person's body (such as kinetic or mechanical energy from body motion, electromagnetic energy from the person's body, blood flow or other internal fluid flow, glucose metabolism, or thermal energy from the person's body).

In addition to at least one imaging member (e.g. camera) and optical sensor (e.g. spectroscopic optical sensor), a device can also comprise one or more sensors selected from the group consisting of: accelerometer (single or multiple axis), chemical sensor, chewing sensor, cholesterol sensor, electrogoniometer or strain gauge, electromagnetic sensor, EMG sensor, glucose sensor, infrared sensor, miniature microphone, motion sensor, pulse sensor, skin galvanic response (Galvanic Skin Response) sensor, sodium sensor, sound sensor, speech recognition sensor, swallowing sensor, temperature sensor, thermometer, and ultrasound sensor.

### Quantifying Close Proximity:

In an example, close proximity can be defined as being less than three inches away. In an example, close proximity can be defined as being less than six inches away from the surface of a person's body. In an example, close proximity can be defined as being less than one inch away from the surface of a person's body.

Imaging Member on the Wrist, Finger, Hand, and/or Arm:

In an example, one or more attachment mechanisms can be configured to hold at least one imaging member in close proximity to a person's wrist, finger, hand, and/or arm. In an example, a device can comprise one or more imaging members worn on a body member selected from the group consisting of: wrist, hand, finger, upper arm, and lower arm. In various examples, one or more attachment mechanisms can be selected from the group consisting of: wrist watch; bracelet; arm band; and finger ring.

In an example, this device and method can comprise an imaging member that is worn on a person's finger in a manner similar to wearing a finger ring, such that the imaging member automatically takes pictures of the person's mouth, a reachable food source, or both as the person moves their arm and hand as the person eats. In an example, a device or system can be worn on, or attached to, one or more parts of a person's body that are selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

Imaging Member on or within a Wrist Band, Bracelet, and/or Smart Watch:

In an example, one or more attachment mechanisms can comprise a wrist band, bracelet, and/or smart watch which is configured to hold at least one imaging member on a person's wrist. In an example, one or more imaging members can be integrated into one or more wearable members that appear similar to a wrist watch, wrist band, bracelet, arm band, necklace, pendant, brooch, collar, eyeglasses, ear ring, headband, or ear-mounted bluetooth device. In an example, one or more attachment mechanisms can be selected from the group consisting of: wrist watch; bracelet; arm band; and finger ring. In an example, a device can comprise one or more imaging members that are worn in a manner similar to a wearable member selected from the group consisting of: wrist watch; bracelet; arm band; and finger ring. In an example, an imaging member can be a smart watch.

In an example, a device and system for measuring a person's consumption of at least one selected type of food, ingredient, or nutrient can take pictures of food using a device selected from the group consisting of: smart watch, smart bracelet, fitness watch, fitness bracelet, watch phone, bracelet phone, wrist band, or other wrist-worn device; arm bracelet; and smart ring or finger ring. In an example, a device can look similar to an attractive wrist watch, bracelet, finger ring, necklace, or ear ring. In an example, a device or system can be worn on, or attached to, one or more parts of a person's body that are selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

In an example, a device can comprise two imaging members. A first imaging member can be worn on a person's wrist like a wrist watch. In an example, two cameras can be worn on the narrow sides of a person's wrist, between the posterior and anterior surfaces of the wrist, such that the moving field of vision from the first of these cameras automatically encompasses the person's mouth (as the person moves their arm when they eat) and the moving field of vision from the second of these cameras automatically encompasses the reachable food source (as the person moves their arm when they eat). This example is comparable to a (conventional) wrist-watch that has been rotated 90 degrees around the person's wrist, with a first camera located where the watch face would be and a second camera located on the opposite side of the wrist.

In an example, a device for measuring a person's consumption can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

### Imaging Member on the Anterior/Palmar/Lower Side or a Lateral/Narrow Side of the Wrist:

In an example, one or more attachment mechanisms can comprise a wrist band, bracelet, and/or smart watch which is configured to hold at least one imaging member on the anterior/palmar/lower side or a lateral/narrow side of a person's wrist for imaging nearby food. In an example, this device can comprise a camera that is worn on the anterior surface of a person's wrist or upper arm, in a manner similar to wearing a (conventional) watch or bracelet that is rotated approximately 180 degrees. In another example, this device can comprise an imaging member with a camera that is worn on the narrow side of a person's wrist or upper arm, in a manner similar to wearing a (conventional) watch or bracelet that is rotated approximately 90 degrees.

In an example, a device can have two cameras attached to a wrist band on opposite (narrow) sides of the person's wrist. In an example, two cameras can be worn on the narrow sides of a person's wrist, between the posterior and anterior surfaces of the wrist. This example is comparable to a (conventional) wrist-watch that has been rotated 90 degrees around the person's wrist, with a first camera located where the (conventional) watch face would be and a second camera located on the opposite side of the wrist.

### Imaging Member Around the Neck or on the Head:

In an example, one or more attachment mechanisms can be configured to hold at least one imaging member in close proximity to a person's neck or head. In an example, a system and device can include one or more imaging members that are worn on a body member selected from the group consisting of: neck; head; and torso. In an example, a device or system can be worn on, or attached to, one or more parts of a person's body that are selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

Imaging Member on or within a Necklace:

In an example, one or more attachment mechanisms can comprise a neck-encircling member which is configured to hold at least one imaging member in proximity to a person's neck. In an example, one or more imaging members can be integrated into one or more wearable members that appear similar to a wrist watch, wrist band, bracelet, arm band, necklace, pendant, brooch, collar, eyeglasses, ear ring, headband, or ear-mounted bluetooth device. In an example, this device can look similar to an attractive wrist watch, bracelet, finger ring, necklace, or ear ring. In an example, a device and system for measuring a person's consumption of at least one selected type of food, ingredient, or nutrient can take pictures of food using a device selected from the group consisting of: smart necklace, smart beads, smart button, neck chain, and neck pendant. In an example, a device can comprise an electronically-functional necklace.

In an example, a device for measuring a person's food consumption can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

In an example, a device can include one or more imaging members that are worn in a manner similar to a wearable member selected from the group consisting of: necklace; pendant, dog tags; brooch; cuff link; ear ring; eyeglasses; wearable mouth microphone; and hearing aid. In an example, a device or system can comprise two imaging members. One imaging member can be worn on a person's neck like a necklace.

Imaging Member on or within Eyewear:

In an example, one or more attachment mechanisms can comprise eyewear which is configured to hold at least one imaging member in proximity to a person's head. In an example, a device can include one or more imaging members that are worn in a manner similar to a wearable member selected from the group consisting of: necklace; pendant, dog tags; brooch; cuff link; ear ring; eyeglasses; wearable mouth microphone; and hearing aid. In an example, a device can comprise a device selected from the group consisting of: smart glasses, visor, or other eyewear; electronically-functional glasses, visor, or other eyewear; augmented reality glasses, visor, or other eyewear; virtual reality glasses, visor, or other eyewear; and electronically-functional contact lens. In an example, an imaging member can be electronically-functional eyewear.

In an example, a device for measuring a person's food consumption can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

Optical Sensor on the Wrist, Finger, Hand, and/or Arm:

In an example, one or more attachment mechanisms can be configured to hold an optical sensor in close proximity to a person's wrist, finger, hand, and/or arm. In an example, one or more attachment mechanisms can be configured to hold a spectroscopic optical sensor in close proximity to a person's wrist, finger, hand, and/or arm. In an example, a wearable sensor can be worn on a person's wrist, hand, finger, and/or arm. In various examples, a sensor can be worn on a person in a location selected from the group consisting of: wrist, neck, finger, hand, head, ear, eyes, nose, teeth, mouth, torso, chest, waist, and leg. In an example, a wearable sensor can be part of an electronically-functional wrist band or smart watch. In an example, a device or system can be worn on, or attached to, one or more parts of a person's body that are selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

Optical Sensor on or within a Wrist Band, Bracelet, and/or Smart Watch:

In an example, one or more attachment mechanisms can comprise a wrist band, bracelet, and/or smart watch which is configured to hold an optical sensor on a person's wrist. In an example, one or more attachment mechanisms can comprise a wrist band, bracelet, and/or smart watch which is configured to hold a spectroscopic optical sensor on a person's wrist. In an example, a wearable sensor can be part of an electronically-functional wrist band or smart watch. In an example, this device can look similar to an attractive wrist watch, bracelet, finger ring, necklace, or ear ring.

In various examples, a wearable sensor can be worn on a person in a manner like a clothing accessory or piece of jewelry selected from the group consisting of: wristwatch, wristphone, wristband, bracelet, cufflink, armband, armlet, and finger ring; necklace, neck chain, pendant, dog tags, locket, amulet, necklace phone, and medallion; eyewear, eyeglasses, spectacles, sunglasses, contact lens, goggles, monocle, and visor; clip, tie clip, pin, brooch, clothing button, and pin-type button; headband, hair pin, headphones, ear phones, hearing aid, earring; and dental appliance, palatal vault attachment, and nose ring.

In an example, a device for measuring a person's consumption can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

Optical Sensor on the Dorsal (or Posterior) Side or the Lateral Side of the Wrist:

In an example, one or more attachment mechanisms can comprise a wrist band, bracelet, and/or smart watch which is configured to hold an optical sensor on the anterior/palmar/lower side or a lateral/narrow side of a person's wrist for scanning nearby food. In an example, one or more attachment mechanisms can comprise a wrist band, bracelet, and/or smart watch which is configured to hold a spectroscopic optical sensor on the anterior/palmar/lower side or a lateral/narrow side of a person's wrist for easier scanning of nearby food. In an example, a wearable sensor can be part of an electronically-functional wrist band or smart watch. In an example, this device can look similar to an attractive wrist watch, bracelet, finger ring, necklace, or ear ring.

### Projected Light-Based Fiducial Marker:

In an example, this system and device further can comprise a light-emitting member which projects a light-based fiducial marker on, or in proximity to, nearby food to estimate food size. In an example, an object of known size can be used as a fiducial marker in order to measure the size or scale of food. In an example, a laser beam can be projected to create a virtual or optical fiducial marker in order to measure food size or scale.

In an example, the volume of food consumed can be estimated by analyzing one or more pictures of that food. In an example, volume estimation can include the use of a physical or virtual fiducial marker or object of known size for estimating the size of a portion of food. In an example, a physical fiducial marker can be placed in the field of view of an imaging system for use as a point of reference or a measure. In an example, this fiducial marker can be a plate, utensil, or other physical place setting member of known size. In an example, this fiducial marker can be created virtually by the projection of coherent light beams. In an example, a device can project (laser) light points onto food and, in conjunction with infrared reflection or focal adjustment, use those points to create a virtual fiducial marker. A fiducial marker may be used in conjunction with a distance-finding mechanism (such as infrared range finder) that determines the distance from the camera and the food.

### Method Embodiment for Food Identification and Quantification:

In an example, a device can be embodied in a method for food identification and quantification comprising the following steps: taking pictures and/or recording images of nearby food using at least one imaging member which is worn in proximity to a person's body; collecting data concerning the spectrum of light that is transmitted through and/or reflected from nearby food using at least one optical sensor which is worn in proximity to a person's body; and automatically analyzing the food pictures and/or images in order to identify the types and quantities of food, ingredients, and/or nutrients using an image-analyzing member.

In various examples, one or more methods to analyze pictures (in order to estimate the types and quantities of food consumed) can be selected from the group consisting of: pattern recognition; food recognition; word recognition; logo recognition; bar code recognition; face recognition; gesture recognition; and human motion recognition. In various examples, a picture of the person's mouth and/or nearby food can be analyzed with one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling.

### Device, System, or Method for Food Identification and Nutritional Intake Modification:

In an example, a wearable device or system for food identification and quantification can comprise: at least one imaging member, wherein this imaging member takes pictures and/or records images of nearby food, and wherein these food pictures and/or images are automatically analyzed to identify the types and quantities of food; an optical sensor, wherein this optical sensor collects data concerning light that is transmitted through or reflected from nearby food, and wherein this data is automatically analyzed to identify the types of food, the types of ingredients in the food, and/or the types of nutrients in the food; one or more attachment mechanisms, wherein these one or more attachment mechanisms are configured to hold the imaging member and the optical sensor in close proximity to the surface of a person's body; an image-analyzing member which automatically analyzes food pictures and/or images; and a computer-to-human interface which modifies the person's nutritional intake.

In an example, a computer-to-human interface can passively provide a person with information concerning food which can modify the person's eating behavior and food consumption. In an example, a computer-to-human interface can provide information to discourage a person from eating unhealthy food and/or encourage a person to eat healthy food. In an example, food can be identified as unhealthy or healthy using the definitions disclosed herein elsewhere.

In an example, a computer-to-human interface can provide information and/or feedback concerning nearby food. In an example, a computer-to-human interface can provide information and/or feedback concerning food that a person is ordering or purchasing. In an example, a computer-to-human interface can provide information and/or feedback concerning food that a person is consuming. In an example, a computer-to-human interface can provide information and/or feedback concerning food that a person has consumed.

In an example, a computer-to-human interface can modify a person's nutritional intake by actively modifying the person's eating behavior, food consumption, and/or nutritional absorption from consumed food. In an example, a computer-to-human interface can be used to not just provide information concerning eating behavior, but also to change a person's eating behavior in a more-active manner. In an example, a food-consumption monitoring device can be in wireless communication with a separate device that modifies a person's eating behavior in a more-active manner. In an example, a computer-to-human interface can comprise one or more mechanisms which actively change a person's food consumption and/or nutritional intake from consumed food.

In an example, a computer-to-human interface can provide a person with one or more stimuli related to food consumption, wherein these stimuli are selected from the group consisting of: auditory feedback (such as a voice message, alarm, buzzer, ring tone, or song); feedback via computer-generated speech; mild external electric charge or neural stimulation; periodic feedback at a selected time of the day or week; phantom taste or smell; phone call; pre-recorded audio or video message by the person from an earlier time; television-based messages; and tactile, vibratory, or pressure-based feedback.

In an example, a computer-to-human interface can create neural stimulation in order to modify a person's eating behavior and/or nutritional intake. In an example, a wearable device can be in wireless communication with a separate device which creates neural stimulation in order to modify a person's eating behavior and/or nutritional intake. In an example, a wearable device and a neural-stimulation implanted device can together comprise a system for modification of nutritional intake.

In an example, a computer-to-human interface can create pressure in order to modify a person's eating behavior and/or nutritional intake. In an example, a wearable device can be in wireless communication with a separate device which creates pressure in order to modify a person's eating behavior and/or nutritional intake. In an example, a wearable device and a pressure-generating device can together comprise a system for modification of nutritional intake.

In an example, a computer-to-human interface can create a phantom taste or smell in order to modify a person's eating behavior and/or nutritional intake. In an example, a wearable device can be in wireless communication with a separate device which creates a phantom taste or smell in order to modify a person's eating behavior and/or nutritional intake. In an example, a wearable device and a taste-or-smell-creating device can together comprise a system for modification of nutritional intake.

In an example, a computer-to-human interface can create an auditory stimulus in order to modify a person's eating behavior and/or nutritional intake. In an example, a wearable device can be in wireless communication with a separate device which creates an auditory stimulus in order to modify a person's eating behavior and/or nutritional intake. In an example, a wearable device and a sound-producing device can together comprise a system for modification of nutritional intake.

In an example, a computer-to-human interface can create a mild external electric charge in order to modify a person's eating behavior and/or nutritional intake. In an example, a wearable device can be in wireless communication with a separate device which creates an electrical charge in order to modify a person's eating behavior and/or nutritional intake. In an example, a wearable device and a charge-generating device can together comprise a system for modification of nutritional intake.

In an example, a computer-to-human interface can create an augmented reality image in order to modify a person's eating behavior and/or nutritional intake. In an example, a wearable device can be in wireless communication with a separate device which creates an augmented reality image in order to modify a person's eating behavior and/or nutritional intake. In an example, an augmented reality image can be displayed in proximity to food in a person's field of view.

In an example, information from a food-consumption monitoring device that measures a person's consumption of at least one selected type of food, ingredient, and/or nutrient can be combined with a computer-to-human interface that provides feedback to encourage the person to eat healthy foods and to limit excess consumption of unhealthy foods. In an example, a food-consumption monitoring device can be in wireless communication with a separate feedback device that modifies a person's eating behavior. In an example, capability for monitoring food consumption can be combined with capability for providing behavior-modifying feedback within a single device. In an example, a single device can be used to measure the selected types and amounts of foods, ingredients, and/or nutrients that a person consumes and to provide visual, auditory, tactile, or other feedback to encourage the person to eat in a healthier manner.

In an example, a device can comprise a computer-to-human interface which modifies a person's nutritional intake based on the types and quantities of foods, ingredients, and/or nutrients consumed by the person. In an example, a computer-to-human interface can modify a person's nutritional intake by modifying the type and/or amount of food which the person consumes. In an example, a computer-to-human interface can modify a person's nutritional intake by modifying the absorption of nutrients from food which the person consumes.

In an example, a computer-to-human interface can reduce a person's consumption of an unhealthy type and/or quantity of food. In an example, a computer-to-human interface can reduce a person's absorption of nutrients from an unhealthy type and/or quantity of food which the person has consumed. In an example, a computer-to-human interface can allow normal (or encourage additional) consumption of a healthy type and/or quantity of food. In an example, a computer-to-human interface can allow normal absorption of nutrients from a healthy type and/or quantity of food which a person has consumed.

In an example, a type of food can be identified as being unhealthy based on analysis of images from an imaging device, analysis of data from one or more wearable sensors, analysis of data from one or more implanted sensors, or a combination thereof. In an example, unhealthy food can be identified as having a high amount or concentration of one or more nutrients selected from the group consisting of: sugars, simple sugars, simple carbohydrates, fats, saturated fats, cholesterol, and sodium. In an example, unhealthy food can be identified as having an amount of one or more nutrients selected from the group consisting of sugars, simple sugars, simple carbohydrates, fats, saturated fats, cholesterol, and sodium that is more than the recommended amount of such nutrient for the person during a given period of time.

In an example, a quantity of food or nutrient which is identified as being unhealthy can be based on one or more factors selected from the group consisting of: the type of food or nutrient; the specificity or breadth of the selected food or nutrient type; the accuracy of a sensor in detecting the selected food or nutrient; the speed or pace of food or nutrient consumption; a person's age, gender, and/or weight; changes in a person's weight; a person's diagnosed health conditions; one or more general health status indicators; the magnitude and/or certainty of the effects of past consumption of the selected nutrient on a person's health; achievement of a person's health goals; a person's exercise patterns and/or caloric expenditure; a person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of a person's consumption of healthy food or nutrients; a dietary plan created for a person by a health care provider; and the severity of a food allergy.

In an example, a computer-to-human interface can be part of a wearable device. In an example, a computer-to-human interface can be part of a wrist band, bracelet, or smart watch. In an example, a computer-to-human interface can be part of electronically-functional eyewear. In an example, a computer-to-human interface can be part of an implanted device which is in electronic communication with a wearable device. In an example, a computer-to-human interface can be a hardware component. In an example, a computer-to-human interface can be a software component.

In an example, a computer-to-human interface can provide feedback to a person and its effect on nutritional intake can depend on the person voluntarily changing their behavior in response to this feedback. In an example, a computer-to-human interface can directly modify the consumption and/or absorption of nutrients in a manner which does not rely on voluntary changes in a person's behavior.

In an example, a computer-to-human interface can provide negative stimuli in association with unhealthy types and quantities of food and/or provide positive stimuli in association with healthy types and quantities of food. In an example, a computer-to-human interface can allow normal absorption of nutrients from healthy types and/or quantities of food, but reduce absorption of nutrients from unhealthy types and/or quantities of food.

In an example, a computer-to-human interface can allow normal absorption of nutrients from a healthy type of food in a person's gastrointestinal tract, but can reduce absorption of nutrients from an unhealthy type of food by releasing an absorption-affecting substance into the person's gastrointestinal tract when the person consumes an unhealthy type of food. In an example, a computer-to-human interface can allow normal absorption of nutrients from a healthy quantity of food in a person's gastrointestinal tract, but can reduce absorption of nutrients from an unhealthy quantity of food by releasing an absorption-affecting substance into the person's gastrointestinal tract when the person consumes an unhealthy quantity of food.

In an example, a computer-to-human interface can reduce absorption of nutrients from an unhealthy type and/or quantity of consumed food by releasing a substance which coats the food as it passes through a person's gastrointestinal tract. In an example, a computer-to-human interface can reduce absorption of nutrients from an unhealthy type and/or quantity of consumed food by releasing a substance which coats a portion of the person's gastrointestinal tract as (or before) that food passes through the person's gastrointestinal tract. In an example, a computer-to-human interface can reduce absorption of nutrients from an unhealthy type and/or quantity of consumed food by releasing a substance which increases the speed with which that food passes through a portion of the person's gastrointestinal tract.

In an example, a computer-to-human interface can comprise an implanted reservoir of a food absorption affecting substance which is released in a person's gastrointestinal tract when the person consumes an unhealthy type and/or quantity of food. In an example, the amount of substance which is released degree to which absorption of food through a person's gastrointestinal tract can be remotely adjusted based on the degree to which a type and/or quantity of consumed food is identified as being unhealthy for that person. In an example, a computer-to-human interface can reduce consumption and/or absorption of nutrients from unhealthy types and/or quantities of food by releasing an absorption-reducing substance into the person's gastrointestinal tract.

In an example, a computer-to-human interface can allow normal consumption and absorption of healthy food, but can reduce a person's consumption and/or absorption of unhealthy food by delivering electromagnetic energy to a portion of the person's gastrointestinal tract (and/or to nerves which innervate that portion of the person's gastrointestinal tract) when the person consumes unhealthy food. In an example, a computer-to-human interface can allow normal consumption and absorption of a healthy quantity of food, but can reduce a person's consumption and/or absorption of an unhealthy quantity of food by delivering electromagnetic energy to a portion of the person's gastrointestinal tract (and/or to nerves which innervate that portion of the person's gastrointestinal tract) when the person consumes an unhealthy quantity of food.

In an example, a computer-to-human interface can deliver electromagnetic energy to a person's stomach and/or to a nerve which innervates the person's stomach. In an example, delivery of electromagnetic energy to a nerve can decrease transmission of natural impulses through that nerve. In an example, delivery of electromagnetic energy to a nerve can simulate natural impulse transmissions through that nerve. In an example, delivery of electromagnetic energy to a person's stomach or associated nerve can cause a feeling of satiety which, in turn, causes the person to consume less food. In an example, delivery of electromagnetic energy to a person's stomach or associated nerve can cause a feeling of nausea which, in turn, causes the person to consume less food.

In an example, delivery of electromagnetic energy to a person's stomach can interfere with the stomach's preparation to receive food, thereby causing the person to consume less food. In an example, delivery of electromagnetic energy to a person's stomach can slow the passage of food through a person's stomach, thereby causing the person to consume less food. In an example, delivery of electromagnetic energy to a person's stomach can interfere with the stomach's preparation to digest food, thereby causing less absorption of nutrients from consumed food. In an example, delivery of electromagnetic energy to a person's stomach can accelerate passage of food through a person's stomach, thereby causing less absorption of nutrients from consumed food. In an example, delivery of electromagnetic energy to a person's stomach can interfere with a person's sensory enjoyment of food and thus cause the person to consume less food.

In an example, a computer-to-human interface can comprise a gastric electric stimulator (GES). In an example, a computer-to-human interface can deliver electromagnetic energy to the wall of a person's stomach. In an example, a computer-to-human interface can be a neurostimulation device. In an example, a computer-to-human interface can be a neuroblocking device. In an example, a computer-to-human interface can stimulate, simulate, block, or otherwise modify electromagnetic signals in a peripheral nervous system pathway.

In an example, a computer-to-human interface can deliver electromagnetic energy to the vagus nerve. In an example, the magnitude and/or pattern of electromagnetic energy which is delivered to a person's stomach (and/or to a nerve which innervates the person's stomach) can be adjusted based on the degree to which a type and/or quantity of consumed food is identified as being unhealthy for that person. Selective interference with the consumption and/or absorption of unhealthy food (versus normal consumption and absorption of healthy food) is an advantage over food-blind gastric stimulation devices and methods in the prior art. In an example, a computer-to-human interface can reduce consumption and/or absorption of nutrients from unhealthy types and/or quantities of food by delivering electromagnetic energy to a portion of the person's gastrointestinal tract and/or to nerves which innervate that portion.

In an example, a computer-to-human interface can allow normal sensory perception of a healthy type of food, but can modify sensory perception of unhealthy food by delivering electromagnetic energy to nerves which innervate a person's tongue and/or nasal passages when the person consumes an unhealthy type of food. In an example, a computer-to-human interface can allow normal sensory perception of a healthy quantity of food, but can modify sensory perception of an unhealthy quantity of food by delivering electromagnetic energy to nerves which innervate a person's tongue and/or nasal passages when the person consumes an unhealthy quantity of food.

In an example, a computer-to-human interface can cause a person to experience an unpleasant virtual taste and/or smell when the person consumes an unhealthy type or quantity of food by delivering electromagnetic energy to afferent nerves which innervate a person's tongue and/or nasal passages. In an example, a computer-to-human interface can cause temporary dysgeusia when a person consumes an unhealthy type or quantity of food. In an example, a computer-to-human interface can cause a person to experience reduced taste and/or smell when the person consumes an unhealthy type or quantity of food by delivering electromagnetic energy to afferent nerves which innervate a person's tongue and/or nose. In an example, a computer-to-human interface can cause temporary ageusia when a person consumes an unhealthy type or quantity of food.

In an example, a computer-to-human interface can stimulate, simulate, block, or otherwise modify electromagnetic signals in an afferent nerve pathway that conveys taste and/or smell information to the brain. In an example, electromagnetic energy can be delivered to synapses between taste receptors and afferent neurons. In an example, a computer-to-human interface can deliver electromagnetic energy to a person's CN VII (Facial Nerve), CN IX (Glossopharyngeal Nerve) CN X (Vagus Nerve), and/or CN V (Trigeminal Nerve). In an example, a computer-to-human interface can inhibit or block the afferent nerves which are associated with selected T1R receptors in order to diminish or eliminate a person's perception of sweetness. In an example, a computer-to-human interface can stimulate or excite the afferent nerves which are associated with T2R receptors in order to create a virtual or phantom bitter taste.

In an example, a computer-to-human interface can deliver a selected pattern of electromagnetic energy to afferent nerves in order to make unhealthy food taste and/or smell bad. In an example, a computer-to-human interface can deliver a selected pattern of electromagnetic energy to afferent nerves in order to make healthy food taste and/or smell good. In an example, the magnitude and/or pattern of electromagnetic energy which is delivered to an afferent nerve can be adjusted based on the degree to which a type and/or quantity of consumed food is identified as being unhealthy for that person. In an example, a computer-to-human interface can reduce consumption and/or absorption of nutrients from unhealthy types and/or quantities of food by delivering electromagnetic energy to nerves which innervate a person's tongue and/or nasal passages.

In an example, a computer-to-human interface can allow normal sensory perception of a healthy type of food, but can modify the taste and/or smell of an unhealthy type of food by releasing a taste and/or smell modifying substance into a person's oral cavity and/or nasal passages. In an example, a computer-to-human interface can allow normal sensory perception of a healthy quantity of food, but can modify the taste and/or smell of an unhealthy quantity of food by releasing a taste and/or smell modifying substance into a person's oral cavity and/or nasal passages. In an example, a computer-to-human interface can release a substance with a strong flavor into a person's oral cavity when the person consumes an unhealthy type and/or quantity of food. In an example, a computer-to-human interface can release a substance with a strong smell into a person's nasal passages when the person consumes an unhealthy type and/or quantity of food. In an example, the release of a taste-modifying or smell-modifying substance can be triggered based on analysis of the type and/or quantity of food consumed.

In an example, a taste-modifying substance can be contained in a reservoir which is attached or implanted within a person's oral cavity. In an example, a taste-modifying substance can be contained in a reservoir which is attached to a person's upper palate. In an example, a taste-modifying substance can be contained in a reservoir within a dental appliance or a dental implant. In an example, a taste-modifying substance can be contained in a reservoir which is implanted so as to be in fluid or gaseous communication with a person's oral cavity. In an example, a smell-modifying substance can be contained in a reservoir which is attached or implanted within a person's nasal passages. In an example, a smell-modifying substance can be contained in a reservoir which is implanted so as to be in gaseous or fluid communication with a person's nasal passages.

In an example, a taste-modifying substance can have a strong flavor which overpowers the natural flavor of food when the substance is released into a person's oral cavity. In an example, a taste-modifying substance can be bitter, sour, hot, or just plain noxious. In an example, a taste-modifying substance can anesthetize or otherwise reduce the taste-sensing function of taste buds on a person's tongue. In an example, a taste-modifying substance can cause temporary ageusia. In an example, a smell-modifying substance can have a strong smell which overpowers the natural smell of food when the substance is released into a person's nasal passages. In an example, a smell-modifying substance can anesthetize or otherwise reduce the smell-sensing function of olfactory receptors in a person's nasal passages. In an example, a computer-to-human interface can reduce consumption and/or absorption of nutrients from unhealthy types and/or quantities of food by releasing a taste and/or smell modifying substance into a person's oral cavity and/or nasal passages.

In an example, a computer-to-human interface can modify a person's food consumption by sending a communication or message to the person wearing the device and/or to another person. In an example, a computer-to-human interface can display information on a wearable or mobile device, send a text, make a phone call, or initiate another form of electronic communication regarding food that is near a person and/or consumed food. In an example, a computer-to-human interface can display information on a wearable or mobile device, send a text, make a phone call, or initiate another form of electronic communication when a person is near food, purchasing food, ordering food, preparing food, and/or consuming food. In an example, information concerning a person's food consumption can be stored in a remote computing device, such as via the internet, and be available for the person to view.

In an example, a computer-to-human interface can send a communication or message to a person who is wearing a device. In an example, a computer-to-human interface can send the person nutritional information concerning food that the person is near, food that the person is purchasing, food that the person is ordering, and/or food that the person is consuming. This nutritional information can include food ingredients, nutrients, and/or calories. In an example, a computer-to-human interface can send the person information concerning the likely health effects of consuming food that the person is near, food that the person is purchasing, food that the person is ordering, and/or food that the person has already starting consuming. In an example, food information which is communicated to the person can be in text form. In an example, a communication can recommend a healthier substitute for unhealthy food which the person is considering consuming.

In an example, food information which is communicated to the person can be in graphic form. In an example, food information which is communicated to the person can be in spoken and/or voice form. In an example, a communication can be in a person's own voice. In an example, a communication can be a pre-recorded message from the person. In an example, a communication can be in the voice of a person who is significant to the person wearing a device. In an example, a communication can be a pre-recorded message from that significant person. In an example, a communication can provide negative feedback in association with consumption of unhealthy food. In an example, a communication can provide positive feedback in association with consumption of healthy food and/or avoiding consumption of unhealthy food. In an example, negative information associated with unhealthy food can encourage the person to eat less unhealthy food and positive information associated with healthy foods can encourage the person to eat more healthy food.

In an example, a computer-to-human interface can send a communication to a person other than the person who is wearing a device. In an example, this other person can provide encouragement and support for the person wearing the device to eat less unhealthy food and/or eat more healthy food. In an example, this other person can be a friend, support group member, family member, or a health care provider. In an example, this device could send a text to Kevin Bacon, or someone who knows him, or someone who knows someone who knows him. In an example, a computer-to-human interface can comprise connectivity with a social network website and/or an internet-based support group. In an example, a computer-to-human interface can encourage a person to reduce consumption of unhealthy types and/or quantities of food (and increase consumption of healthy food) in order to achieve personal health goals. In an example, a computer-to-human interface can encourage a person to reduce consumption of unhealthy types and/or quantities of food (and increase consumption of healthy food) in order to compete with friends and/or people in a peer group with respect to achievement of health goals. In an example, a computer-to-human interface can function as a virtual dietary health coach. In an example, a computer-to-human interface can reduce consumption and/or absorption of nutrients from unhealthy types and/or quantities of food by constricting, slowing, and/or reducing passage of food through the person's gastrointestinal tract.

In an example, a computer-to-human interface can display images or other visual information in a person's field of view which modify the person's consumption of food. In an example, a computer-to-human interface can display images or other visual information in proximity to food in the person's field of view in a manner which modifies the person's consumption of that food. In an example, a computer-to-human interface can be part of an augmented reality system which displays virtual images and/or information in proximity to real world objects. In an example, a nutritional intake modification system can superimpose virtual images and/or information on food in a person's field of view.

In an example, a computer-to-human interface can display virtual nutrition information concerning food that is in a person's field of view. In an example, a computer-to-human interface can display information concerning the ingredients, nutrients, and/or calories in a portion of food which is within a person's field of view. In an example, this information can be based on analysis of images from the imaging device, one or more (other) wearable sensors, or both. In an example, virtual nutrition information can be displayed on a screen (or other display mode) which is separate from a person's view of their environment.

In an example, virtual nutrition information can be superimposed on a person's view of their environment as part of an augmented reality system. In an augmented reality system, virtual nutrition information can be superimposed directly over the food in question. In an example, display of negative nutritional information and/or information about the potential negative effects of unhealthy nutrients can reduce a person's consumption of an unhealthy type or quantity of food. In an example, a computer-to-human interface can display warnings about potential negative health effects and/or allergic reactions. In an example, display of positive nutritional information and/or information on the potential positive effects of healthy nutrients can increase a person's consumption of healthy food. In an example, a computer-to-human interface can display encouraging information about potential health benefits of selected foods or nutrients.

In an example, a computer-to-human interface can display virtual images in response to food that is in a person's field of view. In an example, virtual images can be displayed on a screen (or other display mode) which is separate from a person's view of their environment. In an example, virtual images can be superimposed on a person's view of their environment, such as part of an augmented reality system. In an augmented reality system, a virtual image can be superimposed directly over the food in question. In an example, display of unpleasant image (or one with negative connotations) can reduce a person's consumption of an unhealthy type or quantity of food. In an example, display of an appealing image (or one with positive connotations) can increase a person's consumption of healthy food. In an example, a computer-to-human interface can display an image of a virtual person in response to food, wherein the weight, size, shape, and/or health status of this person is based on the potential effects of (repeatedly) consuming this food. In an example, this virtual person can be a modified version of the person wearing a device, wherein the modification is based on the potential effects of (repeatedly) consuming the food in question. In an example, a device can show the person how they will probably look if they (repeatedly) consume this type and/or quantity of food.

In an example, a computer-to-human interface can be part of an augmented reality system which changes a person's visual perception of unhealthy food to make it less appealing and/or changes the person's visual perception of healthy food to make it more appealing. In an example, a change in visual perception of food can be selected from the group consisting of: a change in perceived color and/or light spectrum; a change in perceived texture or shading; and a change in perceived size or shape. In an example, a computer-to-human interface can display an unappealing image which is unrelated to food but which, when shown in juxtaposition with unhealthy food, will decrease the appeal of that food by association. In an example, a computer-to-human interface can display an appealing image which is unrelated to food but which, when shown in juxtaposition with healthy food, will increase the appeal of that food by association. In an example, a computer-to-human interface can reduce consumption and/or absorption of nutrients from unhealthy types and/or quantities of food by displaying images or other visual information in a person's field of view.

In an example, a computer-to-human interface can allow normal passage of a healthy type of food through a person's gastrointestinal tract, but can constrict, slow, and/or reduce passage of an unhealthy type of food through the person's gastrointestinal tract. In an example, a computer-to-human interface can allow normal passage of up to a healthy cumulative quantity of food (during a meal or selected period of time) through a person's gastrointestinal tract, but can constrict, slow, and/or reduce passage of food in excess of this quantity. In an example, a type and/or quantity of food can be identified as healthy or unhealthy based on analysis of images from the imaging member. In an example, a type and/or quantity of food can be identified as unhealthy based on analysis of images from an imaging device, analysis of data from one or more wearable or implanted sensors, or both. In an example, unhealthy food can be identified as having large (relative) quantities of simple sugars, carbohydrates, saturated fats, bad cholesterol, and/or sodium compounds.

In an example, a computer-to-human interface can selectively constrict, slow, and/or reduce passage of food through a person's gastrointestinal tract by adjustably constricting or resisting jaw movement, adjustably changing the size or shape of the person's oral cavity, adjustably changing the size or shape of the entrance to a person's stomach, adjustably changing the size, shape, or function of the pyloric sphincter, and/or adjustably changing the size or shape of the person's stomach. In an example, such adjustment can be done in a non-invasive (such as through wireless communication) and reversible manner after an operation in which a device is implanted. In an example, the degree to which passage of food through a person's gastrointestinal tract is constricted, slowed, and/or reduced can be adjusted based on the degree to which a type and/or quantity of food is identified as being unhealthy for that person.

In an example, a computer-to-human interface can allow normal absorption of nutrients from consumed food which is identified as a healthy type of food, but can reduce absorption of nutrients from consumed food which is identified as an unhealthy type of food. In an example, a computer-to-human interface can allow normal absorption of nutrients from consumed food up to a selected cumulative quantity (during a meal or selected period of time) which is identified as a healthy quantity of food, but can reduce absorption of nutrients from consumed food greater than this selected cumulative quantity. In an example, a type and/or quantity of food can be identified as healthy or unhealthy based on analysis of images from the imaging member. In an example, a type and/or quantity of food can be identified as unhealthy based on analysis of images from an imaging device, analysis of data from one or more wearable or implanted sensors, or both. In an example, unhealthy food can be identified as having large (relative) quantities of simple sugars, carbohydrates, saturated fats, bad cholesterol, and/or sodium compounds.

In an example, a computer-to-human interface can selectively reduce absorption of nutrients from consumed food by changing the route through which that food passes as that food travels through the person's gastrointestinal tract. In an example, a computer-to-human interface can comprise an adjustable valve within a person's gastrointestinal tract. In an example, an adjustable valve of an intake modification component can be located within a person's stomach. In an example, an adjustable food valve can have a first configuration which directs food through a first route through a person's gastrointestinal tract and can have a second configuration which directs food through a second configuration in a person's gastrointestinal tract. In an example, the first configuration can be shorter or bypass key nutrient-absorbing structures (such as the duodenum) in the gastrointestinal tract. In an example, a computer-to-human interface can direct a healthy type and/or quantity of food through a longer route through a person's gastrointestinal tract and can direct an unhealthy type and/or quantity of food through a shorter route through a person's gastrointestinal tract. In an example, a computer-to-human interface can reduce consumption and/or absorption of nutrients from unhealthy types and/or quantities of food by sending a communication to the person wearing the imaging member and/or to another person.

In an example, a computer-to-human interface can comprise one or more actuators which exert inward pressure on the exterior surface of a person's body in response to consumption of an unhealthy type and/or quantity of food. In an example a computer-to-human interface can comprise one or more actuators which are incorporated into an article of clothing or a clothing accessory, wherein these one or more actuators are constricted when a person consumes an unhealthy type and/or amount of food. In an example, an article of clothing can be smart shirt. In an example, a clothing accessory can be a belt. In an example, an actuator can be a piezoelectric actuator. In an example, an actuator can be a piezoelectric textile or fabric.

In an example, a computer-to-human interface can deliver a low level of electromagnetic energy to the exterior surface of a person's body in response to consumption of an unhealthy type and/or quantity of food. In an example, this electromagnetic energy can act as an adverse stimulus which reduces a person's consumption of unhealthy food. In an example, this electromagnetic energy can interfere with the preparation of the stomach to receive and digest. In an example, a computer-to-human interface can comprise a financial restriction function which impedes the purchase of an unhealthy type and/or quantity of food. In an example, a device can reduce the ability of a person to purchase or order food when the food is identified as being unhealthy.

In an example, a computer-to-human interface can be implanted so as to deliver electromagnetic energy to one or more organs or body tissues selected from the group consisting of: brain, pyloric sphincter, small intestine, large intestine, liver, pancreas, and spleen. In an example, a computer-to-human interface can be implanted so as to deliver electromagnetic energy to the muscles which move one or more organs or body tissues selected from the group consisting of: esophagus, stomach, pyloric sphincter, small intestine, large intestine, liver, pancreas, and spleen. In an example, a computer-to-human interface can be implanted so as to deliver electromagnetic energy to the nerves which innervate one or more organs or body tissues selected from the group consisting of: esophagus, stomach, pyloric sphincter, small intestine, large intestine, liver, pancreas, and spleen.

In an example, a computer-to-human interface can comprise an implanted or wearable drug dispensing device which dispenses an appetite and/or digestion modifying drug in response to consumption of an unhealthy type and/or quantity of food. In an example, a computer-to-human interface can comprise a light-based computer-to-human interface which emits light in response to consumption of an unhealthy type and/or quantity of food. In an example, this interface can comprise an LED array. In an example, a computer-to-human interface can comprise a sound-based computer-to-human interface which emits sound in response to consumption of an unhealthy type and/or quantity of food. In an example, this sound can be a voice, tones, and/or music. In an example, a computer-to-human interface can comprise a tactile-based computer-to-human interface which creates tactile sensations in response to consumption of an unhealthy type and/or quantity of food. In an example, this tactile sensation can be a vibration.

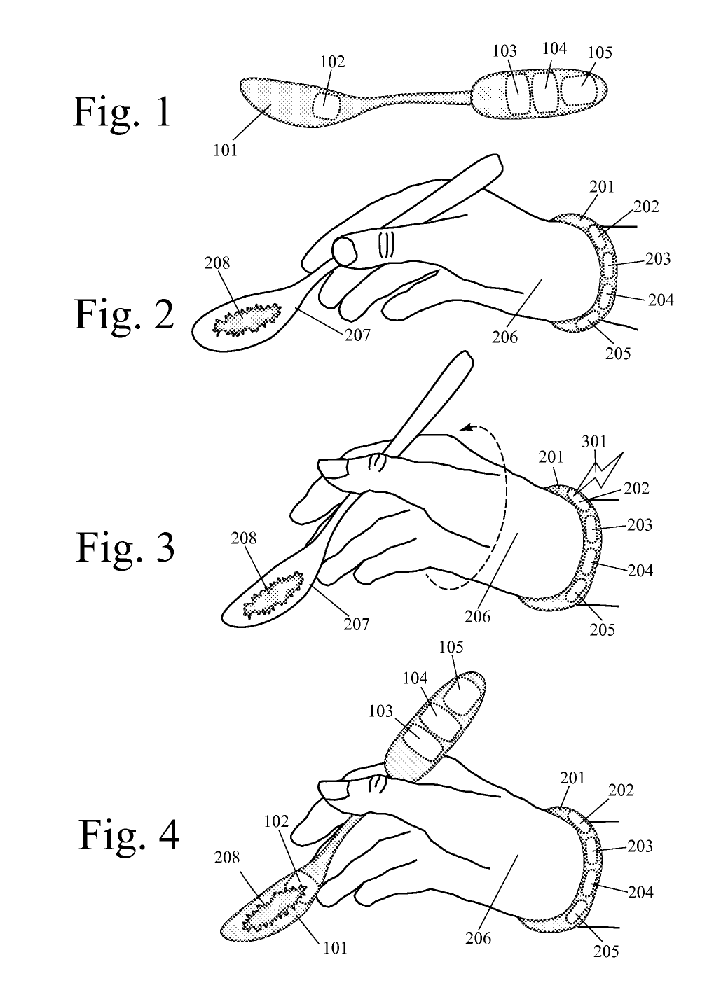

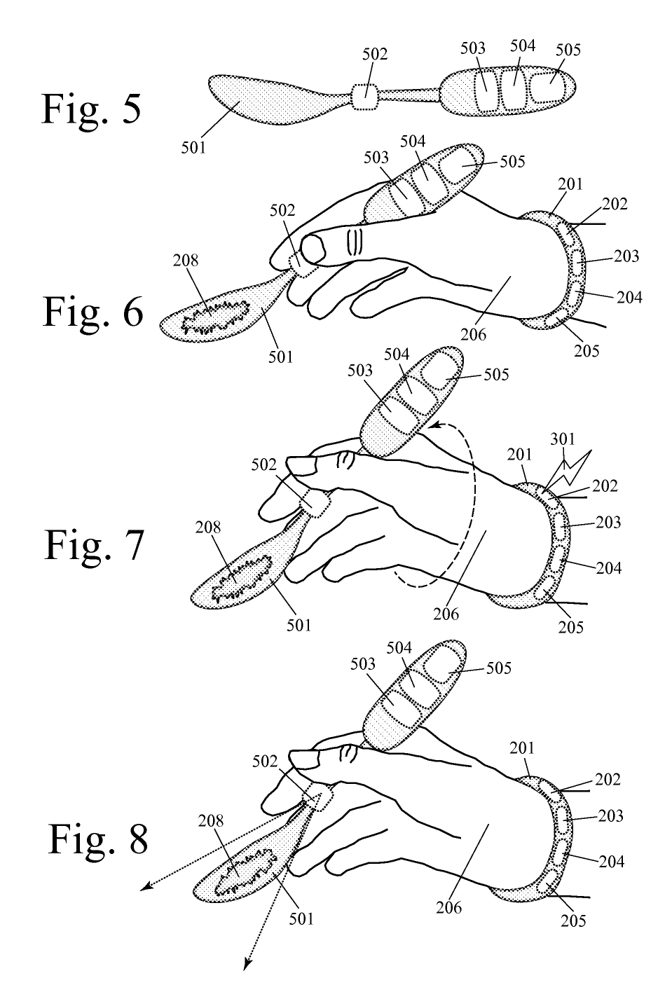

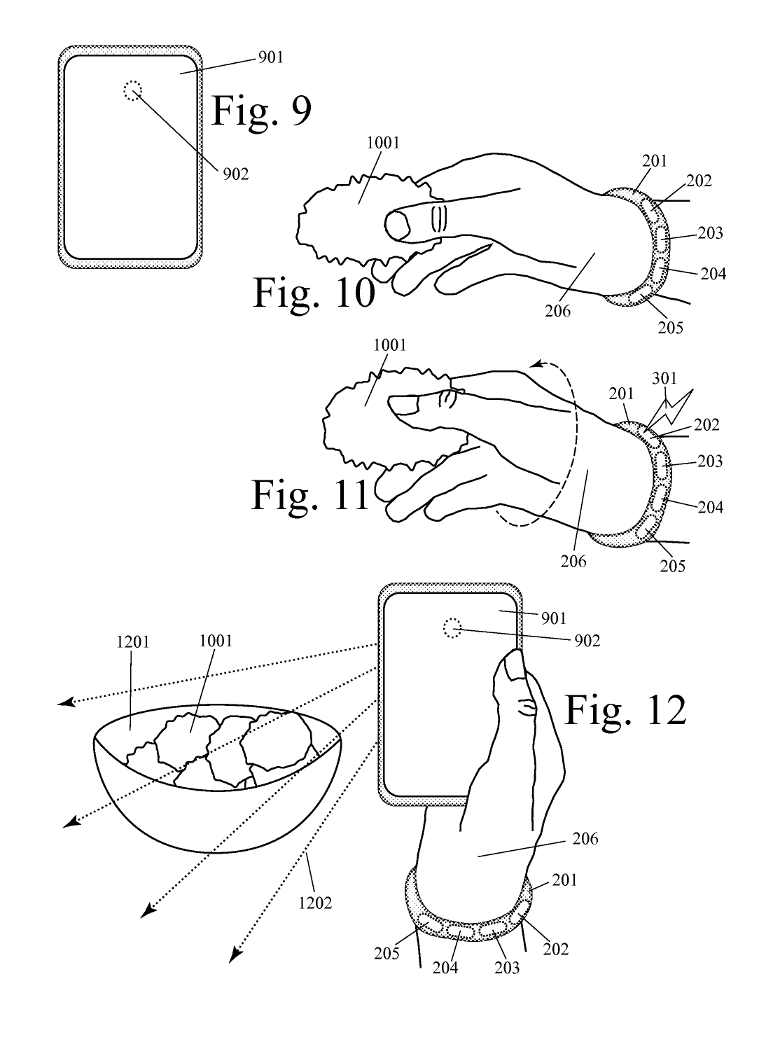

### Narrative to Accompany FIGS. 1 Through 4:

First we will provide an introductory overview to FIGS. 1 through 4. FIGS. 1 through 4 show an example of how a device can be embodied in a device and system for measuring a person's consumption of at least one specific type of food, ingredient, or nutrient, wherein this device and system has two components. The first component is a wearable food-consumption monitor that is worn on a person's body or clothing. In this example, the wearable food-consumption monitor is a smart watch that is worn on a person's wrist. The smart watch automatically collects primary data that is used to detect when a person is consuming food. The second component is a hand-held food-identifying sensor. In this example, the hand-held food-identifying sensor is a smart spoon. The smart spoon collects secondary data that is used to identify the person's consumption of at least one specific type of food, ingredient, or nutrient.

In the example shown in FIGS. 1 through 4, the smart watch collects primary data automatically, without requiring any specific action by the person in association with a specific eating event apart from the actual act of eating. As long as the person continues to wear the smart watch, the smart watch collects the primary data that is used to detect food consumption. In an example, primary data can be motion data concerning the person's wrist movements. In an example, primary data can be up-and-down and tilting movements of the wrist that are generally associated with eating food. In contrast to primary data collection by the smart watch, which is automatic and relatively-continuous, secondary data collection by the smart spoon depends on the person using that particular spoon to eat. In other words, secondary data collection by the smart spoon requires specific action by the person in association with a specific eating event apart from the actual act of eating.

This device and system includes both a smart watch and a smart spoon that work together as an integrated system. Having the smart watch and smart spoon work together provides advantages over use of either a smart watch or a smart spoon by itself. The smart watch provides superior capability for food consumption monitoring (as compared to a smart spoon) because the person wears the smart watch all the time and the smart watch monitors for food consumption continually. The smart spoon provides superior capability for food identification (as compared to a smart watch) because the spoon has direct contact with the food and can directly analyze the chemical composition of food in a manner that is difficult to do with a wrist-worn member. Having both the smart watch and smart spoon work together as an integrated system can provide better monitoring compliance and more-accurate food identification than either working alone.

As FIGS. 1 through 4 collectively show, an integrated device and system that comprises both a smart watch and a smart spoon, working together, can measure a person's consumption of at least one selected type of food, ingredient, or nutrient in a more consistent and accurate manner than either a smart watch or a smart spoon operating alone. One way in which the smart watch and smart spoon can work together is for the smart watch to track whether or not the smart spoon is being used when the smart watch detects that the person is eating food. If the smart spoon is not being used when the person eats, then the smart watch can prompt the person to use the smart spoon. This prompt can range from a relatively-innocuous tone or vibration (which the person can easily ignore) to a more-substantive aversive stimulus, depending on the strength of the person's desire for measurement accuracy and self-control.

Having provided an introductory overview for FIGS. 1 through 4 collectively, we now discuss them individually. FIG. 1 introduces the hand-held food-identifying sensor of this device, which is a smart spoon in this example. In this example, a smart spoon is a specialized electronic spoon that includes food sensors as well as wireless data communication capability. In this example, the smart spoon includes a chemical sensor which analyzes the chemical composition of food with which the spoon comes into contact. FIG. 2 introduces the wearable food-consumption monitor of this device, which is a smart watch in this example. In this example, a smart watch is a wrist-worn electronic device that includes body sensors, a data processing unit, and wireless data communication capability. In this example, the body sensor is a motion sensor. FIGS. 3 and 4 show how the smart spoon and smart watch work together as an integrated system to monitor and measure a person's consumption of at least one selected type of food, ingredient, or nutrient. We now discuss FIGS. 1 through 4 individually in more detail.

FIG. 1 shows that the hand-held food-identifying sensor in this device is a smart spoon 101 that comprises at least four operational components: a chemical composition sensor 102; a data processing unit 103; a communication unit 104; and a power supply and/or transducer 105. In other examples, the hand-held food-identifying sensor component of this device can be a different kind of smart utensil, such as a smart fork, or can be a hand-held food probe. In an example, smart spoon 101 can include other components, such as a motion sensor or camera. The four operational components 102-105 of smart spoon 101 in this example are in electronic communication with each other. In an example, this electronic communication can be wireless. In another example, this electronic communication can be through wires. Connecting electronic components with wires is well-known in the prior art and the precise configuration of possible wires is not central to this invention, so connecting wires are not shown.

In an example, power supply and/or transducer 105 can be selected from the group consisting of: power from a power source that is internal to the device during regular operation (such as an internal battery, capacitor, energy-storing microchip, or wound coil or spring); power that is obtained, harvested, or transduced from a power source other than the person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from the person's body (such as kinetic or mechanical energy from body motion.

In the example shown in FIG. 1, chemical composition sensor 102 on the food-carrying scoop end of smart spoon 101 can identify at least one selected type of food, ingredient, or nutrient by analyzing the chemical composition of food that is carried by smart spoon 101. In this example, chemical composition sensor 102 analyzes the chemical composition of food by being in direct fluid communication with food that is carried in the scoop end of smart spoon 101. In this example, chemical composition sensor 102 includes at least one chemical receptor to which chemicals in a selected type of food, ingredient, or nutrient bind. This binding action creates a signal that is detected by the chemical composition sensor 102, received by the data processing unit 103, and then transmitted to a smart watch or other location via communication unit 104.

In another example, chemical composition sensor 102 can analyze the chemical composition of food by measuring the effects of the interaction between food and light energy. In an example, this interaction can comprise the degree of reflection or absorption of light by food at different light wavelengths. In an example, this interaction can include spectroscopic analysis.

In an example, chemical composition sensor 102 can directly identify at least one selected type of food by chemical analysis of food contacted by the spoon. In an example, chemical composition sensor 102 can directly identify at least one selected type of ingredient or nutrient by chemical analysis of food. In an example, at least one selected type of ingredient or nutrient can be indentified indirectly by: first identifying a type and amount of food; and then linking that identified food to common types and amounts of ingredients or nutrients, using a database that links specific foods to specific ingredients or nutrients. In various examples, such a food database can be located in the data processing unit 103 of smart spoon 101, in the data processing unit 204 of a smart watch 201, or in an external device with which smart spoon 101 and/or a smart watch 201 are in wireless communication.

In various examples, a selected type of food, ingredient, or nutrient that is identified by chemical composition sensor 102 can be selected from the group consisting of: a specific type of carbohydrate, a class of carbohydrates, or all carbohydrates; a specific type of sugar, a class of sugars, or all sugars; a specific type of fat, a class of fats, or all fats; a specific type of cholesterol, a class of cholesterols, or all cholesterols; a specific type of protein, a class of proteins, or all proteins; a specific type of fiber, a class of fiber, or all fiber; a specific sodium compound, a class of sodium compounds, and all sodium compounds; high-carbohydrate food, high-sugar food, high-fat food, fried food, high-cholesterol food, high-protein food, high-fiber food, and high-sodium food.

In various examples, chemical composition sensor 102 can analyze food composition to identify one or more potential food allergens, toxins, or other substances selected from the group consisting of: ground nuts, tree nuts, dairy products, shell fish, eggs, gluten, pesticides, animal hormones, and antibiotics. In an example, a device can analyze food composition to identify one or more types of food (such as pork) whose consumption is prohibited or discouraged for religious, moral, and/or cultural reasons.

In various examples, chemical composition sensor 102 can be selected from the group of sensors consisting of: receptor-based sensor, enzyme-based sensor, reagent based sensor, antibody-based receptor, biochemical sensor, membrane sensor, pH level sensor, osmolality sensor, nucleic acid-based sensor, or DNA/RNA-based sensor; biomimetic sensor (such as an artificial taste bud or an artificial olfactory sensor), chemiresistor, chemoreceptor sensor, electrochemical sensor, electroosmotic sensor, electrophoresis sensor, or electroporation sensor; specific nutrient sensor (such as a glucose sensor, a cholesterol sensor, a fat sensor, a protein-based sensor, or an amino acid sensor); color sensor, colorimetric sensor, photochemical sensor, chemiluminescence sensor, fluorescence sensor, chromatography sensor (such as an analytical chromatography sensor, a liquid chromatography sensor, or a gas chromatography sensor), spectrometry sensor (such as a mass spectrometry sensor), spectrophotometer sensor, spectral analysis sensor, or spectroscopy sensor (such as a near-infrared spectroscopy sensor); and laboratory-on-a-chip or microcantilever sensor.

In an example, smart spoon 101 can measure the quantities of foods, ingredients, or nutrients consumed as well as the specific types of foods, ingredients, or nutrients consumed. In an example, smart spoon 101 can include a scale which tracks the individual weights (and cumulative weight) of mouthfuls of food carried and/or consumed during an eating event. In an example, smart spoon 101 can approximate the weights of mouthfuls of food carried by the spoon by measuring the effect of those mouthfuls on the motion of the spoon as a whole or the relative motion of one part of the spoon relative to another. In an example, smart spoon 101 can include a motion sensor and/or inertial sensor. In an example, smart spoon 101 can include one or more accelerometers in different, motion-variable locations along the length of the spoon. In an example, smart spoon 101 can include a spring and/or strain gauge between the food-carrying scoop of the spoon and the handle of the spoon. In an example, food weight can estimated by measuring distension of the spring and/or strain gauge as food is brought up to a person's mouth.

In an example, smart spoon 101 can use a motion sensor or an inertial sensor to estimate the weight of the food-carrying scoop of the spoon at a first point in time (such as during an upswing motion as the spoon carries a mouthful of food up to the person's mouth) and also at a second point in time (such as during a downswing motion as the person lowers the spoon from their mouth). In an example, smart spoon 101 can estimate the weight of food actually consumed by calculating the difference in food weights between the first and second points in time. In an example, a device can track cumulative food consumption by tracking the cumulative weights of multiple mouthfuls of (different types of) food during an eating event or during a defined period of time (such as a day or week).

FIG. 2 shows that, in this example, the wearable food-consumption monitor component of the device is a smart watch 201. Smart watch 201 is configured to be worn around the person's wrist, adjoining the person's hand 206. In other examples, the wearable food-consumption monitor component of this device can be embodied in a smart bracelet, smart arm band, or smart finger ring. In this example, smart watch 201 includes four operational components: a communication unit 202; a motion sensor 203; a data processing unit 204; and a power supply and/or transducer 205. In other examples, a wearable food-consumption monitor component of this device can be embodied in a smart necklace. In the case of a smart necklace, monitoring for food consumption would more likely be done with a sound sensor rather than a motion sensor. In the case of a smart necklace, food consumption can be monitored and detected by detecting swallowing and/or chewing sounds, rather than monitoring and detecting hand-to-mouth motions.

The four components 202-205 of smart watch 201 are in electronic communication with each other. In an example, this electronic communication can be wireless. In another example, this electronic communication can be through wires. Connecting electronic components with wires is well-known in the prior art and the precise configuration of possible wires is not central to this invention, so a configuration of connecting wires is not shown.

In an example, power supply and/or transducer 205 can be selected from the group consisting of: power from a power source that is internal to the device during regular operation (such as an internal battery, capacitor, energy-storing microchip, or wound coil or spring); power that is obtained, harvested, or transduced from a power source other than the person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from the person's body (such as kinetic or mechanical energy from body motion.

In an example, motion sensor 203 of smart watch 201 can be selected from the group consisting of: bubble accelerometer, dual-axial accelerometer, electrogoniometer, gyroscope, inclinometer, inertial sensor, multi-axis accelerometer, piezoelectric sensor, piezo-mechanical sensor, pressure sensor, proximity detector, single-axis accelerometer, strain gauge, stretch sensor, and tri-axial accelerometer. In an example, motion sensor 203 can collect primary data concerning movements of a person's wrist, hand, or arm.

In an example, there can be an identifiable pattern of movement that is highly-associated with food consumption. Motion sensor 203 can continuously monitor a person's wrist movements to identify times when this pattern occurs to detect when the person is probably eating. In an example, this movement can include repeated movement of the person's hand 206 up to their mouth. In an example, this movement can include a combination of three-dimensional roll, pitch, and yaw by a person's wrist. In an example, motion sensor 203 can also be used to estimate the quantity of food consumed based on the number of motion cycles. In an example, motion sensor 203 can be also used to estimate the speed of food consumption based on the speed or frequency of motion cycles.

In various examples, movements of a person's body that can be monitored and analyzed can be selected from the group consisting of: hand movements, wrist movements, arm movements, tilting movements, lifting movements, hand-to-mouth movements, angles of rotation in three dimensions around the center of mass known as roll, pitch and yaw, and Fourier Transformation analysis of repeated body member movements.

In various examples, smart watch 201 can include a sensor to monitor for possible food consumption other than a motion sensor. In various examples, smart watch 201 can monitor for possible food consumption using one or more sensors selected from the group consisting of: electrogoniometer or strain gauge; optical sensor, miniature still picture camera, miniature video camera, miniature spectroscopy sensor; sound sensor, miniature microphone, speech recognition software, pulse sensor, ultrasound sensor; electromagnetic sensor, skin galvanic response (Galvanic Skin Response) sensor, EMG sensor, chewing sensor, swallowing sensor; and temperature sensor, thermometer, or infrared sensor.

In addition to smart watch 201 that is worn around the person's wrist, FIG. 2 also shows that the person's hand 206 holding a regular spoon 207 that is carrying a mouthful of food 208. It is important to note that this is a regular spoon 207 (with no sensor or data transmission capability), not the smart spoon 101 that was introduced in FIG. 1. There are multiple possible reasons for use of a regular spoon 207 rather than smart spoon 101. In various examples, the person may simply have forgotten to use the smart spoon, may be intentionally trying to “cheat” on dietary monitoring by not using the smart spoon, or may be in dining setting where they are embarrassed to use the smart spoon.

In any event, if the person continues to use the regular spoon 207 instead of the smart spoon 101, then the device and system will not be able to accurately identify the amounts and types of food that they are eating. If the person were not wearing smart watch 201, then the person could continue eating with regular spoon 207 and the device would be completely blind to the eating event. This would lead to low accuracy and low consistency in measuring food consumption. This highlights the accuracy, consistency, and compliance problems that occur if a device relies only on a hand-held food-identifying sensor (without integration with a wearable food-consumption monitor). FIGS. 3 and 4 show how the embodiment disclosed here, comprising both a wearable food-consumption monitor (smart watch 201) and a hand-held food-identification sensor (smart spoon 101) that work together, can correct these problems.

In FIG. 3, motion sensor 203 of smart watch 201 detects the distinctive pattern of wrist and/or arm movement (represented symbolically by the rotational dotted line arrow around hand 206) that indicates that the person is probably consuming food. In an example, a three-dimensional accelerometer on smart watch 201 can detect a distinctive pattern of upward (hand-up-to-mouth) arm movement, followed by a distinctive pattern of tilting or rolling motion (food-into-mouth) wrist movement, followed by a distinctive pattern of downward (hand-down-from-mouth) movement.

If smart watch 201 detects a distinctive pattern of body movements that indicates that the person is probably eating and smart watch 201 has not yet received food identifying secondary data from the use of smart spoon 101, then smart watch 201 can prompt the person to start using smart spoon 101. In an example, this prompt can be relatively-innocuous and easy for the person to ignore if they wish to ignore it. In an example, this prompt can be a quiet tone, gentle vibration, or modest text message to a mobile phone. In another example, this prompt can be a relatively strong and aversive negative stimulus. In an example, this prompt can be a loud sound, graphic warning, mild electric shock, and/or financial penalty.

In the example shown in FIG. 3, the person is not using smart spoon 101 (as they should). This is detected by smart watch 201, which prompts the person to start using smart spoon 101. In FIG. 3, this prompt 301 is represented by a “lightning bolt symbol”. In this example, the prompt 301 represented by the lightning bolt symbol is a mild vibration. In an example, a prompt 301 can be more substantive and/or adverse. In an example, the prompt 301 can involve a wireless signal that to a mobile phone or other intermediary device. In an example, the prompt to the person be communicated through an intermediary device and result in an automated text message or phone call (through a mobile phone, for example) to the person to prompt them to use the smart spoon.

In an example, communication unit 202 of smart watch 201 comprises a computer-to-human interface. In an example, part of this computer-to-human interface 202 can include having the computer prompt the person to collect secondary data concerning food consumption when primary data indicates that the person is probably consuming food. In various examples, communication unit 202 can use visual, auditory, tactile, electromagnetic, gustatory, and/or olfactory signals to prompt the person to use the hand-held food-identifying sensor (smart spoon 101 in this example) to collect secondary data (food chemical composition data in this example) when primary data (motion data in this example) collected by the smart watch indicates that the person is probably eating and the person has not already collected secondary data in association with a specific eating event.

In this example, the person's response to the prompt 301 from smart watch 201 is entirely voluntary; the person can ignore the prompt and continue eating with a regular spoon 207 if they wish. However, if the person wishes to have a stronger mechanism for self-control and measurement compliance, then the person can select (or adjust) a device to make the prompt stronger and less voluntary. In an example, a stronger prompt can be a graphic display showing the likely impact of excessive food consumption, a mild electric shock, an automatic message to a health care provider, and an automatic message to a supportive friend or accountability partner. In an example, the prompt can comprise playing the latest inane viral video song that is sweeping the internet—which the person finds so annoying that they comply and switch from using regular spoon 207 to using smart spoon 101. The strength of the prompt can depend on how strongly the person feels about self-constraint and self-control in the context of monitoring and modifying their patterns of food consumption.

In an example, even if a person's response to prompt 301 is entirely voluntary and the person ignores prompt 301 to use the smart spoon to collect detailed secondary data concerning the meal or snack that the person is eating, the device can still be aware that a meal or snack has occurred. In this respect, even if the person's response to prompt 301 is voluntary, the overall device and system disclosed herein can still track all eating events. This disclosed device provides greater compliance and measurement information than is likely with a hand-held device only. With a hand-held device only, if the person does not use the hand-held member for a particular eating event, then the device is completely oblivious to that eating event. For example, if a device relies on taking pictures from a smart phone to measure food consumption and a person just keeps the phone in their pocket or purse when they eat a snack or meal, then the device is oblivious to that snack or meal. The device disclosed herein corrects this problem. Even if the person does not respond to the prompt, the device still knows that an eating event has occurred.

In an example, there are other ways by which smart watch 201 can detect if smart spoon 101 is being properly used or not. In an example, both smart watch 201 and smart spoon 101 can have integrated motion sensors (such as paired accelerometers) and their relative motions can be compared. If the movements of smart watch 201 and smart spoon 101 are similar during a time when smart watch 201 detects that the person is probably consuming food, then smart spoon 101 is probably being properly used to consume food. However, if smart spoon is not moving when smart watch 201 detects food consumption, then smart spoon 101 is probably just lying somewhere unused and smart watch 201 can prompt the person to use smart spoon 101.

In a similar manner, there can be a wireless (or non-wireless physical linkage) means of detecting physical proximity between smart watch 201 and smart spoon 101. When the person is eating and the smart spoon 101 is not close to smart watch 201, then smart watch 201 can prompt the person to use smart spoon 101. In an example, physical proximity between smart watch 201 and smart spoon 101 can be detected by electromagnetic signals. In an example, physical proximity between smart watch 201 and smart spoon 101 can be detected by optical signals.

If a person feels very strongly about the need for self-constraint and self-control in the measurement and modification of their food consumption, then a device for measuring consumption of at least one selected type of food, ingredient, or nutrient can be made tamper-resistant. In the example shown in FIGS. 1 through 4, smart watch 201 can include a mechanism for detecting when it is removed from the person's body. This can help make it tamper-resistant. In an example, smart watch 201 can monitor signals related to the person's body selected from the group consisting of: pulse, motion, heat, electromagnetic signals, and proximity to an implanted device. In an example, smart watch 201 can detect when it is been removed from the person's wrist by detecting a lack of motion, lack of a pulse, and/or lack of electromagnetic response from skin. In various examples, smart watch 201 can continually monitor optical, electromagnetic, temperature, pressure, or motion signals that indicate that smart watch 201 is properly worn by a person. In an example, smart watch 201 can trigger feedback if it is removed from the person.

In the final figure of this sequence, FIG. 4 shows that the person has responded positively to prompting signal 301 and has switched from using regular spoon 207 (without food sensing and identification capability) to using smart spoon 101 (with food sensing and identification capability). In FIG. 4, the mouthful of food 208 that is being carried by smart spoon 101 is now in fluid or optical communication with chemical composition sensor 102. This enables identification of at least one selected type of food, ingredient, or nutrient by chemical composition sensor 102 as part of smart spoon 101.

In an example, secondary data concerning the type of food, ingredient, or nutrient carried by smart spoon 101 can be wirelessly transmitted from communication unit 104 on smart spoon 101 to communication unit 202 on smart watch 201. In an example, the data processing unit 204 on smart watch 201 can track the cumulative amount consumed of at least one selected type of food, ingredient, or nutrient. In an example, smart watch 201 can convey this data to an external device, such as through the internet, for cumulative tracking and analysis.

In some respects there can be a tradeoff between the accuracy and consistency of food consumption measurement and a person's privacy. The device disclosed herein offers good accuracy and consistency of food consumption measurement, with relatively-low privacy intrusion. In contrast, consider a first method of measuring food consumption that is based only on voluntary use of a hand-held smart phone or smart utensil, apart from any wearable food consumption monitor. This first method can offer relatively-low privacy intrusion, but the accuracy and consistency of measurement depends completely on the person's remembering to use it each time that the person eats a meal or snack—which can be problematic. Alternatively, consider a second method of measuring food consumption that is based only on a wearable device that continually records video pictures of views (or continually records sounds) around the person. This second method can offer relatively high accuracy and consistency of food consumption measurement, but can be highly intrusive with respect to the person's privacy.

The device disclosed herein provides a good solution to this problem of accuracy vs. privacy and is superior to either the first or second methods discussed above. This embodiment of this device that is shown in FIGS. 1 through 4 comprises a motion-sensing smart watch 201 and a chemical-detecting smart spoon 101 that work together to offer relatively-high food measurement accuracy with relatively-low privacy intrusion. Consistent use of the smart watch 201 does not require that a person remember to carry, pack, or otherwise bring a particular piece of portable electronic equipment like methods that rely exclusively on use of mobile phone or utensil. As long as the person does not remove the smart watch, the smart watch goes with them where ever they go and continually monitors for possible food consumption activity. Also, continually monitoring wrist motion is far less-intrusive with respect to a person's privacy than continually monitoring what the person sees (video monitoring) or hears (sound monitoring).

In this example, a smart watch 201 collects primary data concerning probable food consumption and prompts the person to collect secondary for food identification when primary data indicates that the person is probably eating food and the person has not yet collected secondary data. In this example, primary data is body motion data and secondary data comprises chemical analysis of food. In this example, smart watch 201 is the mechanism for collection of primary data and smart spoon 101 is the mechanism for collection of secondary data. In this example, collection of primary data is automatic, not requiring any action by the person in association with a particular eating event apart from the actual act of eating, but collection of secondary data requires a specific action (using the smart spoon to carry food) in association with a particular eating event apart from the actual act of eating. In this example, this combination of automatic primary data collection and non-automatic secondary data collection combine to provide relatively high-accuracy and high-compliance food consumption measurement with relatively low privacy intrusion. This is an advantage over food consumption devices and methods in the prior art.

In an example, information concerning a person's consumption of at least one selected type of food, ingredient, and/or nutrient can be combined with information from a separate caloric expenditure monitoring device that measures a person's caloric expenditure to comprise an overall system for energy balance, fitness, weight management, and health improvement. In an example, a food-consumption monitoring device (such as this smart watch) can be in wireless communication with a separate fitness monitoring device. In an example, capability for monitoring food consumption can be combined with capability for monitoring caloric expenditure within a single smart watch device. In an example, a smart watch device can be used to measure the types and amounts of food, ingredients, and/or nutrients that a person consumes as well as the types and durations of the calorie-expending activities in which the person engages.

### Narrative to Accompany FIGS. 5 Through 8:

The example that is shown in FIGS. 5 through 8 is similar to the one that was just shown in FIGS. 1 through 4, except that now food is identified by taking pictures of food rather than by chemical analysis of food. In FIGS. 5 through 8, smart spoon 501 of this device and system has a built-in camera 502. In an example, camera 502 can be used to take pictures of a mouthful of food 208 in the scoop portion of smart spoon 501. In another example, camera 502 can be used to take pictures of food before it is apportioned by the spoon, such as when food is still on a plate, in a bowl, or in original packaging. In an example, the types and amounts of food consumed can be identified, in a manner that is at least partially automated, by analysis of food pictures.

Like the example that was just shown in FIGS. 1 through 4, the example that is now shown in FIGS. 5 through 8 shows how a device can be embodied in a device and system for measuring a person's consumption that includes both a wearable food-consumption monitor (a smart watch in this example) and a hand-held food-identifying sensor (a smart spoon in this example). However, in this present example, instead of smart spoon 101 having a chemical composition sensor 102 that analyzes the chemical composition of food, smart spoon 501 has a camera 502 to take plain-light pictures of food. These pictures are then analyzed, in a manner that is at least partially automated, in order to identify the amounts and types of foods, ingredients, and/or nutrients that the person consumes. In an example, these pictures of food can be still-frame pictures. In an example, these pictures can be motion (video) pictures.

We now discuss the components of the example shown in FIGS. 5 through 8 in more detail. In FIG. 5, smart spoon 501 includes camera 502 in addition to a data processing unit 503, a communication unit 504, and a power supply and/or transducer 50. The latter three components are like those in the prior example, but the food-identifying sensor (camera 502 vs. chemical composition sensor 102) is different. In this example, camera 502 is built into smart spoon 501 and is located on the portion of smart spoon 501 between the spoon's scoop and the portion of the handle that is held by the person's hand 206.

In this example, camera 502 can be focuses in different directions as the person moves smart spoon 501. In an example, camera 502 can take a picture of a mouthful of food 208 in the scoop of spoon 501. In an example, camera 502 can be directed to take a picture of food on a plate, in a bowl, or in packaging. In this example, camera 502 is activated by touch. In an example, camera 502 can be activated by voice command or by motion of smart spoon 501.

FIG. 6 shows smart spoon 501 in use for food consumption, along with smart watch 201. Smart watch 201 in this example is like smart watch 201 shown in the previous example in FIGS. 1 through 4. As in the last example, smart watch 201 in FIG. 6 includes communication unit 202, motion sensor 203, data processing unit 204, and power supply and/or transducer 205. As in the last example, when the person starts moving their wrist and arm in the distinctive movements that are associated with food consumption, then these movements are recognized by motion sensor 203 on smart watch 201. This is shown in FIG. 7.

If the person has not already used camera 502 on smart spoon 501 to take pictures of food during a particular eating event detected by smart watch 201, then smart watch 201 prompts the person to take a picture of food using camera 502 on smart spoon 501. In this example, this prompt 301 is represented by a “lightning bolt” symbol in FIG. 7. In this example, the person complies with prompt 301 and activates camera 502 by touch in FIG. 8. In this example, a picture is taken of a mouthful of food 208 in the scoop of smart spoon 501. In another example, the person could aim camera 502 on smart spoon 501 toward food on a plate, food in a bowl, or food packaging to take a picture of food before it is apportioned by spoon 501.

In this example, smart watch 201 collects primary data concerning probable food consumption and prompts the person to collect secondary for food identification when primary data indicates that the person is probably eating food and the person has not yet collected secondary data. In this example, primary data is body motion data and secondary data comprises pictures of food. In this example, smart watch 201 is the mechanism for collecting primary data and smart spoon 101 is the mechanism for collecting secondary data. In this example, collection of primary data is automatic, not requiring any action by the person in association with a particular eating event apart from the actual act of eating, but collection of secondary data requires a specific action (triggering and possibly aiming the camera) in association with a particular eating event apart from the actual act of eating. In this example, automatic primary data collection and non-automatic secondary data collection combine to provide relatively high-accuracy and high-compliance food consumption measurement with relatively low privacy intrusion. This is an advantage over food consumption devices and methods in the prior art.

In an example, this device and system can prompt a person to use smart spoon 501 for eating and once the person is using smart spoon 501 for eating this spoon can automatically take pictures of mouthfuls of food that are in the spoon's scoop. In an example, such automatic picture taking can be triggered by infrared reflection, other optical sensor, pressure sensor, electromagnetic sensor, or other contact sensor in the spoon scoop. In another example, this device can prompt a person to manually trigger camera 502 to take a picture of food in the spoon's scoop. In another example, this device can prompt a person to aim camera 502 toward food on a plate, in a bowl, or in original packaging to take pictures of food before it is apportioned into mouthfuls by the spoon. In an example, food on a plate, in a bowl, or in original packaging can be easier to identify by analysis of its shape, texture, scale, and colors than food apportioned into mouthfuls.

In an example, use of camera 502 in smart spoon 501 can rely on having the person manually aim and trigger the camera for each eating event. In an example, the taking of food pictures in this manner requires at least one specific voluntary human action associated with each food consumption event, apart from the actual act of eating, in order to take pictures of food during that food consumption event. In an example, such specific voluntary human actions can be selected from the group consisting of: bringing smart spoon 501 to a meal or snack; using smart spoon 501 to eat food; aiming camera 502 of smart spoon 501 at food on a plate, in a bowl, or in original packaging; triggering camera 502 by touching a button, screen, or other activation surface; and triggering camera 502 by voice command or gesture command.

In an example, camera 502 of smart spoon 501 can be used to take multiple still-frame pictures of food. In an example, camera 502 of smart spoon 501 can be used to take motion (video) pictures of food from multiple angles. In an example, camera 502 can take pictures of food from at least two different angles in order to better segment a picture of a multi-food meal into different types of foods, better estimate the three-dimensional volume of each type of food, and better control for differences in lighting and shading. In an example, camera 502 can take pictures of food from multiple perspectives to create a virtual three-dimensional model of food in order to determine food volume. In an example, quantities of specific foods can be estimated from pictures of those foods by volumetric analysis of food from multiple perspectives and/or by three-dimensional modeling of food from multiple perspectives.

In an example, pictures of food on a plate, in a bowl, or in packaging can be taken before and after consumption. In an example, the amount of food that a person actually consumes (not just the amount ordered by the person or served to the person) can be estimated by measuring the difference in food volume from pictures before and after consumption. In an example, camera 502 can image or virtually create a fiduciary market to better estimate the size or scale of food. In an example, camera 502 can be used to take pictures of food which include an object of known size. This object can serve as a fiduciary marker in order to estimate the size and/or scale of food. In an example, camera 502, or another component on smart spoon 501, can project light beams within the field of vision to create a virtual fiduciary marker. In an example, pictures can be taken of multiple sequential mouthfuls of food being transported by the scoop of smart spoon 501 and used to estimate the cumulative amount of food consumed.

In an example, there can be a preliminary stage of processing or analysis of food pictures wherein image elements and/or attributes are adjusted, normalized, or standardized. In an example, a food picture can be adjusted, normalized, or standardized before it is compared with food pictures in a food database. This can improve segmentation of a meal into different types of food, identification of foods, and estimation of food volume or mass. In an example, food size or scale can be adjusted, normalized, or standardized before comparison with pictures in a food database. In an example, food texture can be adjusted, normalized, or standardized before comparison with pictures in a food database. In an example, food lighting or shading can be adjusted, normalized, or standardized before comparison with pictures in a food database. In various examples, a preliminary stage of food picture processing and/or analysis can include adjustment, normalization, or standardization of food color, texture, shape, size, context, geographic location, adjacent foods, place setting context, and temperature.

In an example, a food database can be used as part of a device and system for identifying types and amounts of food, ingredients, or nutrients. In an example, a food database can include one or more elements selected from the group consisting of: food name, food picture (individually or in combinations with other foods), food color, food packaging bar code or nutritional label, food packaging or logo pattern, food shape, food texture, food type, common geographic or intra-building locations for serving or consumption, common or standardized ingredients (per serving, per volume, or per weight), common or standardized nutrients (per serving, per volume, or per weight), common or standardized size (per serving), common or standardized number of calories (per serving, per volume, or per weight), common times or special events for serving or consumption, and commonly associated or jointly-served foods.

In an example, the boundaries between different types of food in a picture of a meal can be automatically determined to segment the meal into different food types before comparison with pictures in a food database. In an example, individual portions of different types of food within a multi-food meal can be compared individually with images of portions of different types of food in a food database. In an example, a picture of a meal including multiple types of food can be automatically segmented into portions of different types of food for comparison with different types of food in a food database. In an example, a picture of a meal with multiple types of food can be compared as a whole with pictures of meals with multiple types of food in a food database.

In an example, a food database can also include average amounts of specific ingredients and/or nutrients associated with specific types and amounts of foods for measurement of at least one selected type of ingredient or nutrient. In an example, a food database can be used to identify the type and amount of at least one selected type of ingredient that is associated with an identified type and amount of food. In an example, a food database can be used to identify the type and amount of at least one selected type of nutrient that is associated with an identified type and amount of food. In an example, an ingredient or nutrient can be associated with a type of food on a per-portion, per-volume, or per-weight basis.

In an example, automatic identification of food amounts and types can include extracting a vector of food parameters (such as color, texture, shape, and size) from a food picture and comparing this vector with vectors of these parameters in a food database. In various examples, methods for automatic identification of food types and amounts from food pictures can include: color analysis, image pattern recognition, image segmentation, texture analysis, three-dimensional modeling based on pictures from multiple perspectives, and volumetric analysis based on a fiduciary marker or other object of known size.

In various examples, food pictures can be analyzed in a manner which is at least partially automated in order to identify food types and amounts using one or more methods selected from the group consisting of: analysis of variance; chi-squared analysis; cluster analysis; comparison of a vector of food parameters with a food database containing such parameters; energy balance tracking; factor analysis; Fourier transformation and/or fast Fourier transform (FFT); image attribute adjustment or normalization; pattern recognition; comparison with food images with food images in a food database; inter-food boundary determination and food portion segmentation; linear discriminant analysis; linear regression and/or multivariate linear regression; logistic regression and/or probit analysis; neural network and machine learning; non-linear programming; principal components analysis; scale determination using a physical or virtual fiduciary marker; three-dimensional modeling to estimate food quantity; time series analysis; and volumetric modeling.

In an example, attributes of food in an image can be represented by a multi-dimensional food attribute vector. In an example, this food attribute vector can be statistically compared to the attribute vector of known foods in order to automate food identification. In an example, multivariate analysis can be done to identify the most likely identification category for a particular portion of food in an image. In various examples, a multi-dimensional food attribute vector can include attributes selected from the group consisting of: food color; food texture; food shape; food size or scale; geographic location of selection, purchase, or consumption; timing of day, week, or special event; common food combinations or pairings; image brightness, resolution, or lighting direction; infrared light reflection; spectroscopic analysis; and person-specific historical eating patterns. In an example, in some situations the types and amounts of food can be identified by analysis of bar codes, brand logos, nutritional labels, or other optical patterns on food packaging.

In an example, analysis of data concerning food consumption can include comparison of food consumption parameters between a specific person and a reference population. In an example, data analysis can include analysis of a person's food consumption patterns over time. In an example, such analysis can track the cumulative amount of at least one selected type of food, ingredient, or nutrient that a person consumes during a selected period of time.

In an example, pictures of food can be analyzed within the data processing unit of a hand-held device (such as a smart spoon) or a wearable device (such as a smart watch). In an example, pictures of food can be wirelessly transmitted from a hand-held or wearable device to an external device, wherein these food pictures are automatically analyzed and food identification occurs. In an example, the results of food identification can then be wirelessly transmitted back to the wearable or hand-held device. In an example, identification of the types and quantities of foods, ingredients, or nutrients that a person consumes can be a combination of, or interaction between, automated identification food methods and human-based food identification methods.

In the example shown in FIGS. 5 through 8, food-imaging camera 502 is built into smart spoon 501. In various alternative examples, a device and system for measuring a person's consumption of at least one selected type of food, ingredient, or nutrient can take pictures of food with an imaging device or component that is selected from the group consisting of: smart food utensil and/or electronically-functional utensil, smart spoon, smart fork, food probe, smart chop stick, smart plate, smart dish, or smart glass; smart phone, mobile phone, or cell phone; smart watch, watch cam, smart bracelet, fitness watch, fitness bracelet, watch phone, or bracelet phone; smart necklace, necklace cam, smart beads, smart button, neck chain, or neck pendant; smart finger ring or ring cam; electronically-functional or smart eyewear, smart glasses, visor, augmented or virtual reality glasses, or electronically-functional contact lens; digital camera; and electronic tablet.

### Narrative to Accompany FIGS. 9 Through 12:

The example that is shown in FIGS. 9 through 12 is similar to the one that was just shown in FIGS. 5 through 8, except that now food pictures are taken by a general-purpose mobile electronic device (such as a smart phone) rather than by a specialized food utensil (such as a smart spoon). In this example, the general-purpose mobile electronic device is a smart phone. In other examples, a general-purpose mobile electronic device can be an electronic tablet or a digital camera.

The wearable food-monitoring component of the example shown in FIGS. 9 through 12 is again a smart watch with a motion sensor, like the one in previous examples. The smart watch and smart phone components of this example work together in FIGS. 9 through 12 in a similar manner to the way in which the smart watch and smart spoon components worked together in the example shown in FIGS. 5 through 8. We do not repeat the methodological detail of possible ways to identify food based on food pictures here because this was already discussed in the narrative accompanying the previous example.

FIG. 9 shows a rectangular general-purpose smart phone 901 that includes a camera (or other imaging component) 902. FIG. 10 shows a person grasping food item 1001 in their hand 206. FIG. 10 also shows that this person is wearing a smart watch 201 that includes communication unit 202, motion sensor 203, data processing unit 204, and power supply and/or transducer 205. In an example, food item 1001 can be a deep-fried pork rind. In another example, food item 1001 can be a blob of plain tofu; however, it is unlikely that any person who eats a blob of plain tofu would even need a device like this.

FIG. 11 shows this person bringing food item 1001 up to their mouth with a distinctive rotation of their wrist that is represented by the dotted-line arrow around hand 206. This indicates that the person is probably eating food. Using motion sensor 203, smart watch 201 detects this pattern of movement and detects that the person is probably eating something. Since the person has not yet taken a picture of food in association with this eating event, smart watch 201 prompts the person to take a picture of food using smart phone 901. This prompt 301 is represented in FIG. 11 by a “lightning bolt” symbol coming out from communication unit 202. We discussed a variety of possible prompts in earlier examples and do not repeat them here.

FIG. 12 shows that this person responds positively to prompt 301. This person responds by taking a picture of food items 1001 in bowl 1201 using camera 902 in smart phone 901. The field of vision of camera 902 is represented by dotted-line rays 1202 that radiate from camera 902 toward bowl 1201. In an example, the person manually aims camera 902 of smart phone 901 toward the food source (bowl 1201 in this example) and then triggers camera 902 to take a picture by touching the screen of smart phone 901. In another example, the person could trigger camera 902 with a voice command or a gesture command.

In this example, smart watch 201 and smart phone 901 share wireless communication. In an example, communication with smart watch 201 can be part of a smart phone application that runs on smart phone 901. In an example, smart watch 201 and smart phone 901 can comprise part of an integrated system for monitoring and modifying caloric intake and caloric expenditure to achieve energy balance, weight management, and improved health.

In an example, smart watch 201 and/or smart phone 901 can also be in communication with an external computer. An external computer can provide advanced data analysis, data storage and memory, communication with health care professionals, and/or communication with a support network of friends. In an example, a general purpose smart phone can comprise the computer-to-human interface of a device and system for measuring a person's consumption of at least one selected type of food, ingredient, or nutrient. In an example, such a device and system can communicate with a person by making calls or sending text messages through a smart phone. In an alternative example, an electronic tablet can serve the role of a hand-held imaging and interface device instead of smart phone 901.

FIGS. 9 through 12 show an embodiment of a device for measuring a person's consumption of at least one selected type of food, ingredient, or nutrient comprising a wearable food-consumption monitor (a smart watch in this example) that is configured to be worn on the person's wrist, arm, hand or finger and a hand-held food-identifying sensor (a smart phone in this example). The person is prompted to use the smart phone to take pictures of food when the smart watch indicates that the person is consuming food. In this example, primary data concerning food consumption that is collected by a smart watch includes data concerning movement of the person's body and secondary data for food identification that is collected by a smart phone includes pictures of food. In this example, the person is prompted to take pictures of food when they are moving in a manner that indicates that they are probably eating and secondary data has not already been collected.

The system for measuring food consumption that is shown in FIGS. 9 through 12 combines continual motion monitoring by a smart watch and food imaging by a smart phone. It is superior to prior art that relies only on a smart phone. A system for measuring food consumption that depends only on the person using a smart phone to take a picture of every meal and every snack they eat will probably have much lower compliance and accuracy than the system disclosed herein. With the system disclosed herein, as long as the person wears the smart watch (which can be encouraged by making it comfortable and tamper resistant), the system disclosed herein continually monitors for food consumption. A system based on a stand-alone smart phone offers no such functionality.

Ideally, if the smart watch 201 herein is designed to be sufficiently comfortable and unobtrusive, it can be worn all the time. Accordingly, it can even monitor for night-time snacking. It can monitor food consumption at times when a person would be unlikely to bring out their smart phone to take pictures (at least not without prompting). The food-imaging device and system that is shown here in FIGS. 9 through 12, including the coordinated operation of a motion-sensing smart watch and a wirelessly-linked smart phone, can provide highly-accurate food consumption measurement with relatively-low privacy intrusion.

FIGS. 9 through 12 also show an example of how a device can be embodied in a device for monitoring food consumption comprising: (a) a wearable sensor that is configured to be worn on a person's body or clothing, wherein this wearable sensor automatically collects data that is used to detect probable eating events without requiring action by the person in association with a probable eating event apart from the act of eating, and wherein a probable eating event is a period of time during which the person is probably eating; (b) an imaging member, wherein this imaging member is used by the person to take pictures of food that the person eats, wherein using this imaging member to take pictures of food requires voluntary action by the person apart from the act of eating, and wherein the person is prompted to take pictures of food using this imaging member when data collected by the wearable sensor indicates a probable eating event; and (c) a data analysis component, wherein this component analyzes pictures of food taken by the imaging member to estimate the types and amounts of foods, ingredients, nutrients, and/or calories that are consumed by the person. In this example, the wearable sensor is motion sensor 203. In this example, the imaging member is camera 902 which is part of phone 901. In this example, the data analysis component is data processing unit 204.

In the example shown in FIGS. 9 through 12, motion sensor 203 automatically collects data that is used to detect probable eating events. In this example, this data comprises hand motion. When data collected by motion sensor 203 indicates a probable eating event, then communication unit 202 sends a signal that prompts the person to use imaging member 902 to take pictures of food 1001 which the person is eating. When prompted, the person uses camera 902 to take pictures of food 1001. Then, data analysis component 204 analyzes these food pictures to estimate the types and amounts of foods, ingredients, nutrients, and/or calories that are consumed by the person.

In this example, data analysis occurs in a wrist-based data analysis component. In other examples, analysis of food pictures can occur in other locations. In an example, analysis of food pictures can occur in a data analysis component that is located in phone 901. In another example, analysis of food pictures can occur in a remote computer with which phone 901 or communication unit 202 is in wireless communication.

In the example shown in FIGS. 9 through 12, a wearable sensor is worn on the person's wrist. In other examples, a wearable sensor can be worn on a person's hand, finger, or arm. In this example, a wearable sensor is part of an electronically-functional wrist band or smart watch. In another example, a wearable sensor can be an electronically-functional adhesive patch that is worn on a person's skin. In another example, a sensor can be worn on a person's clothing.

In the example shown in FIGS. 9 through 12, an imaging member is a mobile phone or mobile phone application. In another example, an imaging member can be electronically-functional eyewear. In another example, an imaging member can be a smart watch. In another example, an imaging member can be an electronically-functional necklace. In this example, a wearable sensor and imaging member are separate but in wireless communication with each other. In another example, a wearable sensor and an imaging member can be jointly located, such as in a smart watch, necklace, or eyewear.

In the example shown in FIGS. 9 through 12, a wearable sensor automatically collects data concerning motion of the person's body. In another example, a wearable sensor can automatically collect data concerning electromagnetic energy that is emitted from the person's body or transmitted through the person's body. In another example, a wearable sensor can automatically collect data concerning thermal energy that is emitted from the person's body. In another example, a wearable sensor can automatically collect data concerning light energy that is reflected from the person's body or absorbed by the person's body. In various examples, food events can be detected by monitoring selected from the group consisting of: monitoring motion of the person's body; monitoring electromagnetic energy that is emitted from the person's body or transmitted through the person's body; monitoring thermal energy that is emitted from the person's body; and monitoring light energy that is reflected from the person's body or absorbed by the person's body.

In the example shown in FIGS. 9 through 12, the person is prompted to take pictures of food using the imaging member when data collected by the wearable sensor indicates a probable eating event and the person does not take pictures of food for this probable eating event before or at the start of the probable eating event. In an example, the person can be prompted to take pictures of food using the imaging member when data collected by the wearable sensor indicates a probable eating event and the person does not take pictures of food for this probable eating event before a selected length of time after the start of the probable eating event. In an example, the person can be prompted to take pictures of food using the imaging member when data collected by the wearable sensor indicates a probable eating event and the person does not take pictures of food for this probable eating event before a selected quantity of eating-related actions occurs during the probable eating event. In an example, the person can be prompted to take pictures of food using the imaging member when data collected by the wearable sensor indicates a probable eating event and the person does not take pictures of food for this probable eating event at the end of the probable eating event.

In a variation on this example, a device can be embodied in a device for monitoring food consumption comprising: (a) a wearable sensor that is configured to be worn on a person's wrist, hand, finger, or arm, wherein this wearable sensor automatically collects data that is used to detect probable eating events without requiring action by the person in association with a probable eating event apart from the act of eating, and wherein a probable eating event is a period of time during which the person is probably eating; (b) an imaging member, wherein this imaging member is used by the person to take pictures of food that the person eats, wherein using this imaging member to take pictures of food requires voluntary action by the person apart from the act of eating, and wherein the person is prompted to take pictures of food using this imaging member when data collected by the wearable sensor indicates a probable eating event; and (c) a data analysis component, wherein this component analyzes pictures of food taken by the imaging member to estimate the types and amounts of foods, ingredients, nutrients, and/or calories that are consumed by the person.

In a variation on this example, a device can be embodied in a device for monitoring food consumption comprising: (a) a wearable sensor that is configured to be worn on a person's wrist, hand, finger, or arm, wherein this wearable sensor automatically collects data that is used to detect probable eating events without requiring action by the person in association with a probable eating event apart from the act of eating, wherein a probable eating event is a period of time during which the person is probably eating, and wherein this data is selected from the group consisting of data concerning motion of the person's body, data concerning electromagnetic energy emitted from or transmitted through the person's body, data concerning thermal energy emitted from the person's body, and light energy reflected from or absorbed by the person's body; (b) an imaging member, wherein this imaging member is used by the person to take pictures of food that the person eats, wherein using this imaging member to take pictures of food requires voluntary action by the person apart from the act of eating, wherein the person is prompted to take pictures of food using this imaging member when data collected by the wearable sensor indicates a probable eating event; and (c) a data analysis component, wherein this component analyzes pictures of food taken by the imaging member to estimate the types and amounts of foods, ingredients, nutrients, and/or calories that are consumed by the person, and wherein this component analyzes data received from the sensor and pictures of food taken by the imaging member to evaluate the completeness of pictures taken by the imaging member for tracking the person's total food consumption.

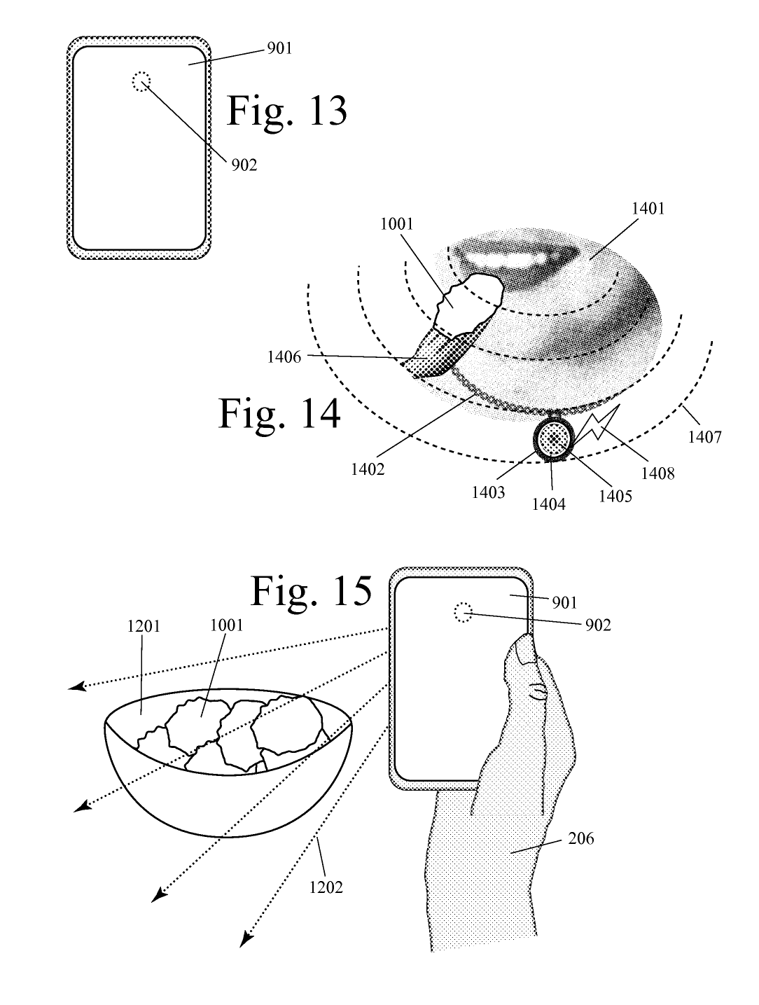

### Narrative to Accompany FIGS. 13 Through 18:

The example that is shown in FIGS. 13 through 15 is similar to the one that was just shown in FIGS. 9 through 12, except that the wearable food-monitoring component is now a smart necklace instead of a smart watch. The smart necklace in this example monitors for food consumption by monitoring sounds instead of motion. In this example, the smart necklace detects food consumption by detecting chewing or swallowing sounds.

FIG. 13 shows the smart phone 901 with camera 902 that was introduced in the previous example. FIG. 14 shows that the person 1401 is wearing smart necklace 1402 including communication unit 1403, data processing unit and power supply 1404, and microphone 1405. FIG. 14 also shows that the person is eating food item 1001 using fork 1406.

In FIG. 14, microphone 1405 of smart necklace 1402 detects that the person is consuming food based on chewing or swallowing sounds. In FIG. 14, chewing or swallowing sounds are represented by dotted-line curves 1407 expanding outwardly from the person's mouth. Smart necklace 1402 then prompts the person to take a picture of food using camera 902 on smart phone 901. In FIG. 14, this prompt 1408 is represented by a “lightning bolt” symbol coming out from communication unit 1403.

FIG. 15 shows that the person responds to prompt 1408 by aiming camera 902 of smart phone 901 toward bowl 1201 containing food items 1001. The field of vision of camera 902 is represented by dotted-line rays 1202 that radiate outwards from camera 902 toward bowl 1201.

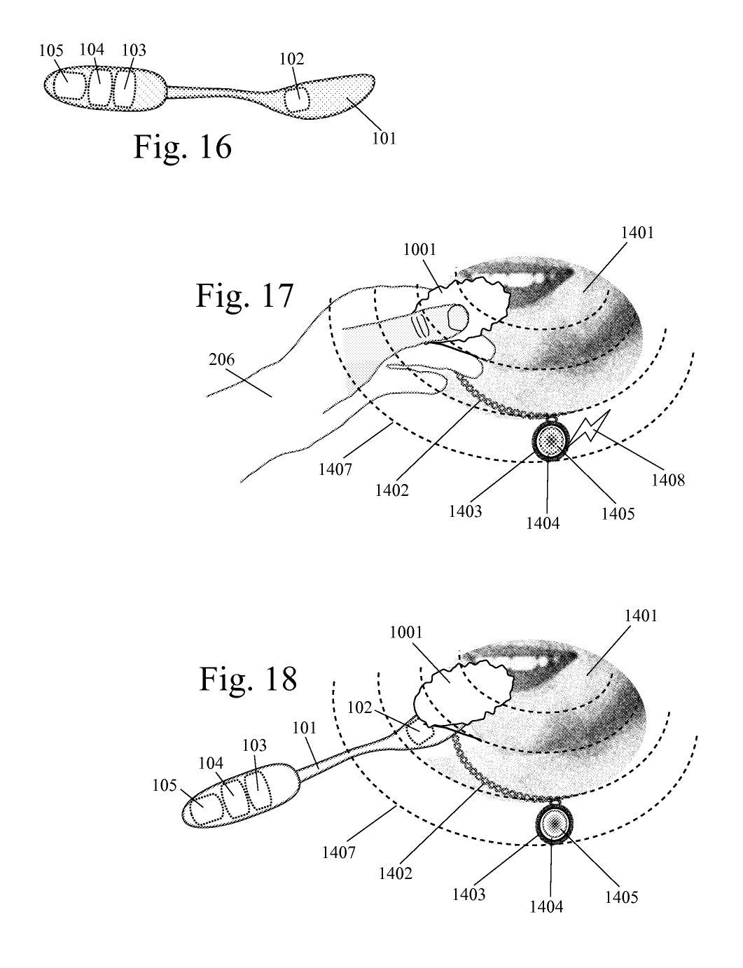

The example that is shown in FIGS. 16 through 18 is similar to the one that was just shown in FIGS. 13 through 15, except that hand-held food-identifying component is the smart spoon that was introduced earlier instead of a smart phone. FIG. 16 shows smart spoon 101 with chemical composition sensor 102, data processing unit 103, communication unit 104, and power supply and/or transducer 105.

FIG. 17 shows that the person is eating food item 1001 without using smart spoon 101. In FIG. 17, microphone 1405 of smart necklace 1402 detects that the person is consuming food based on chewing or swallowing sounds 1407. In FIG. 14, chewing or swallowing sounds are represented by dotted-line curves 1407 expanding outwardly from the person's mouth. Smart necklace 1402 then prompts the person to use smart spoon 101 to eat food item 1001. In FIG. 14, this prompt 1408 is represented by a “lightning bolt” symbol coming out from communication unit 1403.

FIG. 18 shows that the person responds to prompt 1408 by using smart spoon 101. Use of smart spoon 101 brings food item 1001 into contact with chemical composition sensor 102 on smart spoon 101. This contact enables identification of food item 1001.

FIGS. 1 through 18 show various examples of a device for measuring a person's consumption of at least one selected type of food, ingredient, or nutrient comprising: a wearable food-consumption monitor, wherein this food-consumption monitor is configured to be worn on a person's body or clothing, and wherein this food-consumption monitor automatically collects primary data that is used to detect when a person is consuming food, without requiring any specific action by the person in association with a specific eating event with the exception of the act of eating; and a hand-held food-identifying sensor, wherein this food-identifying sensor collects secondary data that is used to identify the person's consumption of at least one selected type of food, ingredient, or nutrient.

In FIGS. 1 through 18, the collection of secondary data by a hand-held food-identifying sensor requires a specific action by the person in association with a specific eating event apart from the act of eating. Also in FIGS. 1 through 18, the person whose food consumption is monitored is prompted to perform a specific action to collect secondary data when primary data collected by a food-consumption monitor indicates that the person is probably eating and the person has not already collected secondary data in association with a specific eating event.

FIGS. 1 through 12 show various examples of a device wherein a wearable food-consumption monitor is a smart watch or smart bracelet. FIGS. 9 through 15 show various examples of a device wherein a hand-held food-identifying sensor is a smart phone, cell phone, or mobile phone. FIGS. 1 through 8 and also FIGS. 16 through 18 show various examples of a device wherein a hand-held food-identifying sensor is a smart fork, smart spoon, other smart utensil, or food probe.

FIGS. 1 through 4 show an example of a device wherein a wearable food-consumption monitor is a smart watch or other electronic member that is configured to be worn on the person's wrist, arm, hand or finger; wherein a hand-held food-identifying sensor is a smart food utensil or food probe; and wherein a person is prompted to use the smart food utensil or food probe to analyze the chemical composition of food when the smart watch indicates that the person is consuming food.

FIGS. 1 through 4 show an example of a device wherein a wearable food-consumption monitor is a smart watch or other electronic member that is configured to be worn on the person's wrist, arm, hand or finger; wherein primary data collected by the smart watch or other electronic member that is configured to be worn on the person's wrist, arm, hand or finger includes data concerning movement of the person's body; wherein a hand-held food-identifying sensor is a smart food utensil or food probe; and wherein a person is prompted to use the smart food utensil or food probe to analyze the chemical composition of food when the smart watch indicates that the person is consuming food.

FIGS. 9 through 12 show an example of a device wherein a wearable food-consumption monitor is a smart watch or other electronic member that is configured to be worn on the person's wrist, arm, hand or finger; wherein a hand-held food-identifying sensor is a smart phone, cell phone, or mobile phone; and wherein a person is prompted to use the smart phone, cell phone, or mobile phone to take pictures of food or food packaging when the smart watch indicates that the person is consuming food.

FIGS. 9 through 12 show an example of a device wherein a wearable food-consumption monitor is a smart watch or other electronic member that is configured to be worn on the person's wrist, arm, hand or finger; wherein primary data collected by the smart watch or other electronic member that is configured to be worn on the person's wrist, arm, hand or finger includes data concerning movement of the person's body; wherein a hand-held food-identifying sensor is a smart phone, cell phone, or mobile phone; and wherein a person is prompted to use the smart phone, cell phone, or mobile phone to take pictures of food or food packaging when primary data indicates that the person is consuming food.

In another example: a wearable food-consumption monitor can be a smart watch or other electronic member that is configured to be worn on the person's wrist, arm, hand or finger wherein primary data collected by the smart watch or other electronic member that is configured to be worn on the person's wrist, arm, hand or finger includes data concerning electromagnetic energy received from the person's body; a hand-held food-identifying sensor can be a smart food utensil or food probe; and a person can be prompted to use the smart food utensil or food probe to analyze the chemical composition of food when the smart watch indicates that the person is consuming food.

In another example: a wearable food-consumption monitor can be a smart watch or other electronic member that is configured to be worn on the person's wrist, arm, hand or finger wherein primary data collected by the smart watch or other electronic member that is configured to be worn on the person's wrist, arm, hand or finger includes data concerning electromagnetic energy received from the person's body; a hand-held food-identifying sensor can be a smart phone, cell phone, or mobile phone; and a person can be prompted to use the smart phone, cell phone, or mobile phone to take pictures of food or food packaging when primary data indicates that the person is consuming food.

In another example: a wearable food-consumption monitor can be a smart watch or other electronic member that is configured to be worn on the person's wrist, arm, hand or finger wherein primary data collected by the smart watch or other electronic member that is configured to be worn on the person's wrist, arm, hand or finger includes images; a hand-held food-identifying sensor can be a smart food utensil or food probe; and a person can be prompted to use the smart food utensil or food probe to analyze the chemical composition of food when the smart watch indicates that the person is consuming food.

In another example: a wearable food-consumption monitor can be a smart watch or other electronic member that is configured to be worn on the person's wrist, arm, hand or finger wherein primary data collected by the smart watch or other electronic member that is configured to be worn on the person's wrist, arm, hand or finger includes images; a hand-held food-identifying sensor can be a smart phone, cell phone, or mobile phone; and a person can be prompted to use the smart phone, cell phone, or mobile phone to take pictures of food or food packaging when primary data indicates that the person is consuming food.

In another example: a wearable food-consumption monitor is a smart necklace or other electronic member that is configured to be worn on the person's neck, head, or torso wherein primary data collected by the smart watch or other electronic member that is configured to be worn on the person's wrist, arm, hand or finger includes patterns of sonic energy; a hand-held food-identifying sensor can be a smart food utensil or food probe; and a person can be prompted to use the smart food utensil or food probe to analyze the chemical composition of food when the smart watch indicates that the person is consuming food.

In another example: a wearable food-consumption monitor is a smart necklace or other electronic member that is configured to be worn on the person's neck, head, or torso wherein primary data collected by the smart watch or other electronic member that is configured to be worn on the person's wrist, arm, hand or finger includes patterns of sonic energy; a hand-held food-identifying sensor can be a smart phone, cell phone, or mobile phone; and a person can be prompted to use the smart phone, cell phone, or mobile phone to take pictures of food or food packaging when primary data indicates that the person is consuming food.

In an example, at least one selected type of food, ingredient, or nutrient for these examples can be selected from the group consisting of: a specific type of carbohydrate, a class of carbohydrates, or all carbohydrates; a specific type of sugar, a class of sugars, or all sugars; a specific type of fat, a class of fats, or all fats; a specific type of cholesterol, a class of cholesterols, or all cholesterols; a specific type of protein, a class of proteins, or all proteins; a specific type of fiber, a class of fiber, or all fiber; a specific sodium compound, a class of sodium compounds, and all sodium compounds; high-carbohydrate food, high-sugar food, high-fat food, fried food, high-cholesterol food, high-protein food, high-fiber food, and high-sodium food.

In an example, at least one selected type of food, ingredient, or nutrient can be selected from the group consisting of: a selected food, ingredient, or nutrient that has been designated as unhealthy by a health care professional organization or by a specific health care provider for a specific person; a selected substance that has been identified as an allergen for a specific person; peanuts, shellfish, or dairy products; a selected substance that has been identified as being addictive for a specific person; alcohol; a vitamin or mineral; vitamin A, vitamin B1, thiamin, vitamin B12, cyanocobalamin, vitamin B2, riboflavin, vitamin C, ascorbic acid, vitamin D, vitamin E, calcium, copper, iodine, iron, magnesium, manganese, niacin, pantothenic acid, phosphorus, potassium, riboflavin, thiamin, and zinc; a specific type of carbohydrate, class of carbohydrates, or all carbohydrates; a specific type of sugar, class of sugars, or all sugars; simple carbohydrates, complex carbohydrates; simple sugars, complex sugars, monosaccharides, glucose, fructose, oligosaccharides, polysaccharides, starch, glycogen, disaccharides, sucrose, lactose, starch, sugar, dextrose, disaccharide, fructose, galactose, glucose, lactose, maltose, monosaccharide, processed sugars, raw sugars, and sucrose; a specific type of fat, class of fats, or all fats; fatty acids, monounsaturated fat, polyunsaturated fat, saturated fat, trans fat, and unsaturated fat; a specific type of cholesterol, a class of cholesterols, or all cholesterols; Low Density Lipoprotein (LDL), High Density Lipoprotein (HDL), Very Low Density Lipoprotein (VLDL), and triglycerides; a specific type of protein, a class of proteins, or all proteins; dairy protein, egg protein, fish protein, fruit protein, grain protein, legume protein, lipoprotein, meat protein, nut protein, poultry protein, tofu protein, vegetable protein, complete protein, incomplete protein, or other amino acids; a specific type of fiber, a class of fiber, or all fiber; dietary fiber, insoluble fiber, soluble fiber, and cellulose; a specific sodium compound, a class of sodium compounds, and all sodium compounds; salt; a specific type of meat, a class of meats, and all meats; a specific type of vegetable, a class of vegetables, and all vegetables; a specific type of fruit, a class of fruits, and all fruits; a specific type of grain, a class of grains, and all grains; high-carbohydrate food, high-sugar food, high-fat food, fried food, high-cholesterol food, high-protein food, high-fiber food, and high-sodium food.

FIGS. 1 through 18 show various examples of a device for measuring a person's consumption of at least one selected type of food, ingredient, or nutrient comprising: (a) a wearable food-consumption monitor, wherein this food-consumption monitor is configured to be worn on a person's body or clothing, and wherein this food-consumption monitor automatically collects primary data that is used to detect when a person is consuming food, without requiring any specific action by the person in association with a specific eating event with the exception of the act of eating; (b) a hand-held food-identifying sensor, wherein this food-identifying sensor collects secondary data that is used to identify the person's consumption of at least one selected type of food, ingredient, or nutrient; wherein collection of secondary data by this hand-held food-identifying sensor requires a specific action by the person in association with a specific eating event apart from the act of eating; and (c) a computer-to-human interface, wherein this interface uses visual, auditory, tactile, electromagnetic, gustatory, and/or olfactory communication to prompt the person to use the hand-held food-identifying sensor to collect secondary data when primary data collected by the food-consumption monitor indicates that the person is probably eating and the person has not already collected secondary data in association with a specific eating event.

FIGS. 1 through 18 also show various examples of a method for measuring a person's consumption of at least one selected type of food, ingredient, or nutrient comprising: (a) automatically collecting primary data using a food-consumption monitor that a person wears on their body or clothing without requiring any specific action by the person in association with a specific eating event with the possible exception of the act of eating, wherein this primary data is used to detect when the person is consuming food; (b) collecting secondary data using a hand-held food-identifying sensor wherein collection of secondary data requires a specific action by the person in association with a specific eating event apart from the act of eating, and wherein this secondary data is used to identify the person's consumption of at least one selected type of food, ingredient, or nutrient; and (c) prompting the person to use a hand-held food-identifying sensor to collect secondary data when primary data collected by a food-consumption monitor indicates that the person is eating and the person has not already collected secondary data in association with a specific eating event.

Figures shown and discussed herein also disclose a device for monitoring food consumption comprising: (a) a wearable sensor that is configured to be worn on a person's body or clothing, wherein this wearable sensor automatically collects data that is used to detect probable eating events without requiring action by the person in association with a probable eating event apart from the act of eating, and wherein a probable eating event is a period of time during which the person is probably eating; (b) an imaging member, wherein this imaging member is used by the person to take pictures of food that the person eats, wherein using this imaging member to take pictures of food requires voluntary action by the person apart from the act of eating, and wherein the person is prompted to take pictures of food using this imaging member when data collected by the wearable sensor indicates a probable eating event; and (c) a data analysis component, wherein this component analyzes pictures of food taken by the imaging member to estimate the types and amounts of foods, ingredients, nutrients, and/or calories that are consumed by the person.

Figures shown and discussed herein disclose a device for monitoring food consumption wherein the wearable sensor is worn on a person's wrist, hand, finger, or arm. Figures shown and discussed herein disclose a device wherein the wearable sensor is part of an electronically-functional wrist band or smart watch. In another example, a wearable sensor can be part of an electronically-functional adhesive patch that is worn on a person's skin.

Figures shown and discussed herein disclose a device for monitoring food consumption wherein the imaging member is a mobile phone or mobile phone application. In another example, the imaging member can be electronically-functional eyewear. In another example, the imaging member can be a smart watch. In another example, the imaging member can be an electronically-functional necklace. In another example, the imaging member can be an electronically-functional wearable button.

Figures shown and discussed herein disclose a device for monitoring food consumption wherein the wearable sensor and the imaging member are in wireless communication with each other. Figures shown and discussed herein disclose a device for monitoring food consumption wherein the wearable sensor automatically collects data concerning motion of the person's body. In another example, the wearable sensor can automatically collect data concerning electromagnetic energy emitted from the person's body or transmitted through the person's body. In another example, the wearable sensor can automatically collect data concerning thermal energy emitted from the person's body. In another example, the wearable sensor can automatically collect data concerning light energy reflected from the person's body or absorbed by the person's body.

Figures shown and discussed herein disclose a device for monitoring food consumption wherein the person is prompted to take pictures of food using the imaging member when data collected by the wearable sensor indicates a probable eating event and the person does not take pictures of food for this probable eating event before or at the start of the probable eating event. In another example, the person can be prompted to take pictures of food using the imaging member when data collected by the wearable sensor indicates a probable eating event and the person does not take pictures of food for this probable eating event before a selected length of time after the start of the probable eating event. In another example, the person can be prompted to take pictures of food using the imaging member when data collected by the wearable sensor indicates a probable eating event and the person does not take pictures of food for this probable eating event before a selected quantity of eating-related actions occurs during the probable eating event. In another example, the person can be prompted to take pictures of food using the imaging member when data collected by the wearable sensor indicates a probable eating event and the person does not take pictures of food for this probable eating event at the end of the probable eating event.

Figures shown and discussed herein also disclose a device for monitoring food consumption comprising: (a) a wearable sensor that is configured to be worn on a person's wrist, hand, finger, or arm, wherein this wearable sensor automatically collects data that is used to detect probable eating events without requiring action by the person in association with a probable eating event apart from the act of eating, and wherein a probable eating event is a period of time during which the person is probably eating; (b) an imaging member, wherein this imaging member is used by the person to take pictures of food that the person eats, wherein using this imaging member to take pictures of food requires voluntary action by the person apart from the act of eating, and wherein the person is prompted to take pictures of food using this imaging member when data collected by the wearable sensor indicates a probable eating event; and (c) a data analysis component, wherein this component analyzes pictures of food taken by the imaging member to estimate the types and amounts of foods, ingredients, nutrients, and/or calories that are consumed by the person.

Figures shown and discussed herein also disclose a device for monitoring food consumption comprising: (a) a wearable sensor that is configured to be worn on a person's wrist, hand, finger, or arm, wherein this wearable sensor automatically collects data that is used to detect probable eating events without requiring action by the person in association with a probable eating event apart from the act of eating, wherein a probable eating event is a period of time during which the person is probably eating, and wherein this data is selected from the group consisting of data concerning motion of the person's body, data concerning electromagnetic energy emitted from or transmitted through the person's body, data concerning thermal energy emitted from the person's body, and light energy reflected from or absorbed by the person's body; (b) an imaging member, wherein this imaging member is used by the person to take pictures of food that the person eats, wherein using this imaging member to take pictures of food requires voluntary action by the person apart from the act of eating, wherein the person is prompted to take pictures of food using this imaging member when data collected by the wearable sensor indicates a probable eating event; and (c) a data analysis component, wherein this component analyzes pictures of food taken by the imaging member to estimate the types and amounts of foods, ingredients, nutrients, and/or calories that are consumed by the person, and wherein this component analyzes data received from the sensor and pictures of food taken by the imaging member to evaluate the completeness of pictures taken by the imaging member for tracking the person's total food consumption.

In an example, a caloric intake measuring system can use spectroscopic and 3D imaging analysis. In an example, a caloric intake measuring system can comprise: a spectroscopic sensor that collects data concerning light that is absorbed by or reflected from food, wherein this food is to be consumed by a person, and wherein this data is used to estimate the composition of this food; and an imaging device that takes images of this food from different angles, wherein these images from different angles are used to estimate the quantity of this food. Information concerning the estimated composition of the food and information concerning the estimated quantity of the food can be combined to estimate the person's caloric intake.

In an example, a caloric intake measuring system can comprise: a spectroscopic sensor that collects data concerning light that is absorbed by or reflected from food, wherein this food is to be consumed by a person, and wherein this data is used to estimate the composition of this food; and an imaging device that takes images of this food from different angles, wherein these images from different angles are used to estimate the quantity of this food. In an example, information concerning the estimated composition of food and information concerning the estimated quantity of food can be combined to estimate a person's caloric intake. In an example, estimation of the composition of food can comprise estimating one or more nutrients or ingredients selected from the group consisting of: a specific type of carbohydrate, a class of carbohydrates, or all carbohydrates; a specific type of sugar, a class of sugars, or all sugars; a specific type of fat, a class of fats, or all fats; a specific type of cholesterol, a class of cholesterols, or all cholesterols; a specific type of protein, a class of proteins, or all proteins; a specific type of fiber, a class of fiber, or all fiber; a specific sodium compound, a class of sodium compounds, and all sodium compounds; high-carbohydrate food, high-sugar food, high-fat food, fried food, high-cholesterol food, high-protein food, high-fiber food, and high-sodium food.

In an example, a spectroscopic sensor can direct a beam of light toward food and analyzes the spectrum of light reflected from the food. In an example, a beam of light can be coherent. In an example, a beam of light can be infrared. In an example, a beam of light can be ultraviolet. In an example, a spectroscopic sensor can be part of a food probe. In an example, a spectroscopic sensor can be a part of a food utensil. In an example, a spectroscopic sensor can be a part of a wearable device which is configured to be worn on a person's wrist, arm, hand, finger, neck, torso, or head. In an example, a spectroscopic sensor can be a part of an electronically-functional watch, wrist-band, bracelet, ring, arm band, necklace, button, piece of eyewear, ear piece, or headband.

In an example, an imaging device can take images of food before and after food consumption and analyze differences between these images to better estimate the net quantity of food actually consumed by a person. In an example, an imaging device can take sequential images of food from different angles. In an example, an imaging device can take simultaneous images of food from different angles. In an example, three-dimensional analysis can be used to estimate the volume of food from images of food taken from different angles. In an example, an imaging device can be part of a food probe or utensil. In an example, an imaging device can be part of a phone. In an example, an imaging device can be part of a wearable device which is configured to be worn on a person's wrist, arm, hand, finger, neck, torso, or head.

In an example, a wearable caloric intake measuring device can comprise: a device that is configured to be worn on a person's body or clothing to measure the person's caloric intake, wherein this device further comprises, a spectroscopic sensor that collects data concerning light that is absorbed by or reflected from food, wherein this food is to be consumed by a person, and wherein this data is used to estimate the nutritional and/or chemical composition of this food; and an imaging component that takes simultaneous or sequential images of this food from different angles, wherein these images from different angles are used to estimate the quantity of this food.

In an example, a portable caloric intake measuring device can comprise: a device that is configured to be held by a person to measure the person's caloric intake, wherein this device further comprises, a spectroscopic sensor that collects data concerning light that is absorbed by or reflected from food, wherein this food is to be consumed by a person, and wherein this data is used to estimate the nutritional and/or chemical composition of this food; and an imaging component that takes simultaneous or sequential images of this food from different angles, wherein these images from different angles are used to estimate the quantity of this food.

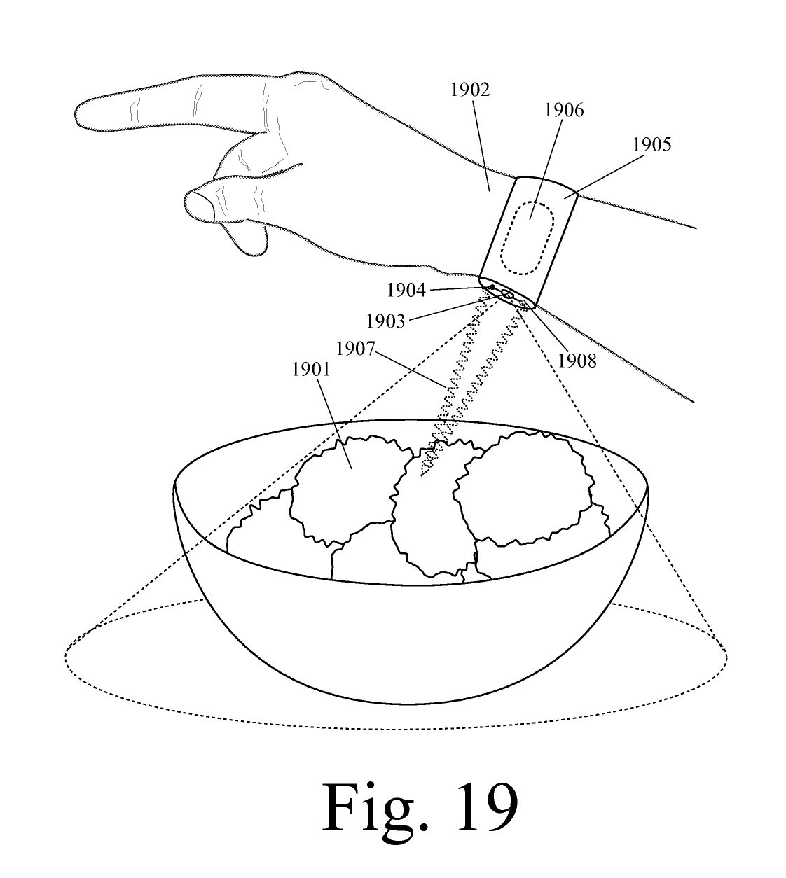

### Narrative to Accompany FIGS. 19 Through 21:

FIGS. 19 through 21 show examples of how a wearable device or system for food identification and quantification can comprise: at least one imaging member, wherein this imaging member takes pictures and/or records images of nearby food, and wherein these food pictures and/or images are automatically analyzed to identify the types and quantities of food; an optical sensor, wherein this optical sensor collects data concerning light that is transmitted through or reflected from nearby food, and wherein this data is automatically analyzed to identify the types of food, the types of ingredients in the food, and/or the types of nutrients in the food; one or more attachment mechanisms, wherein these one or more attachment mechanisms are configured to hold the imaging member and the optical sensor in close proximity to the surface of a person's body; and an image-analyzing member which automatically analyzes food pictures and/or images. The examples shown in FIGS. 19 through 21 can further comprise any of the variations in components or methods which were discussed herein in other sections.

FIG. 19, in particular, shows an example of how a device can be embodied in a wearable device for food identification and quantification comprising: imaging member 1903, wherein imaging member 1903 takes pictures and/or records images of nearby food 1901, and wherein these food pictures and/or images are automatically analyzed to identify the types and quantities of food 1901; optical sensor 1904, wherein optical sensor 1904 collects data concerning light 1907 that is reflected from nearby food 1901, and wherein this data is automatically analyzed to identify the types of food 1901, the types of ingredients in food 1901, and/or the types of nutrients in food 1901; attachment mechanism 1905, wherein attachment mechanism 1905 is configured to hold imaging member 1903 and optical sensor 1904 in close proximity to the surface of a person's body 1902; and image-analyzing member 1906 which automatically analyzes food pictures and/or images.

The example shown in FIG. 19 also includes a light-emitting member 1908 which emits light 1907 which is then reflected from nearby food 1901. In this example, imaging member 1903 is a camera. In this example, imaging member 1903 is configured to have a focal direction which points outward from the surface of the person's body 1902. In this example, optical sensor 1904 is a spectroscopic optical sensor that collects data concerning the spectrum of light 1907 that is reflected from nearby food 1901. In this example, optical sensor 1904 is configured to have a sensing direction which points outward from the surface of the person's body 1902.

In the example shown in FIG. 19, attachment mechanism 1905 is a wrist band. In this example, image-analyzing member 1906 is a data control unit which can further comprise one or more components selected from the group consisting of: data processing unit; motion sensor, electromagnetic sensor, optical sensor, and/or chemical sensor; graphic display component; human-to-computer communication component; memory component; power source; and wireless data transmission and reception component.

In this example, attachment mechanism 1905 is configured to hold imaging member 1903 in close proximity to the person's wrist 1902. In this example, attachment mechanism 1905 comprises a wrist band which is configured to hold imaging member 1903 on the person's wrist 1902. In this example, attachment mechanism 1905 comprises a wrist band which is configured to hold imaging member 1903 on the anterior/palmar/lower side of the person's wrist 1903 in order to easily take pictures and/or record images of nearby food 1901. In this example, close proximity is defined as being less than three inches away. In another example, close proximity can defined as being less than six inches away.

In the example shown in FIG. 19, attachment mechanism 1905 is configured to hold optical sensor 1904 in close proximity to the person's wrist 1902. In this example, attachment mechanism 1905 comprises a wrist band which is configured to hold optical sensor 1904 on the person's wrist 1902. In this example, attachment mechanism 1905 comprises a wrist band which is configured to hold optical sensor 1904 on the anterior/palmar/lower side of the person's wrist 1903 in order to easily sense light 1907 reflected from nearby food 1901.

FIG. 19 shows a device which can support a method for food identification and quantification comprising the following steps: taking pictures and/or recording images of nearby food 1901 using at least one imaging member 1904 which is worn in proximity to a person's body 1902; collecting data concerning the spectrum of light 1907 that is transmitted through and/or reflected from nearby food 1901 using at least one optical sensor 1904 which is worn in proximity to a person's body 1902; and automatically analyzing the food pictures and/or images in order to identify the types and quantities of food, ingredients, and/or nutrients using an image-analyzing member 1906.

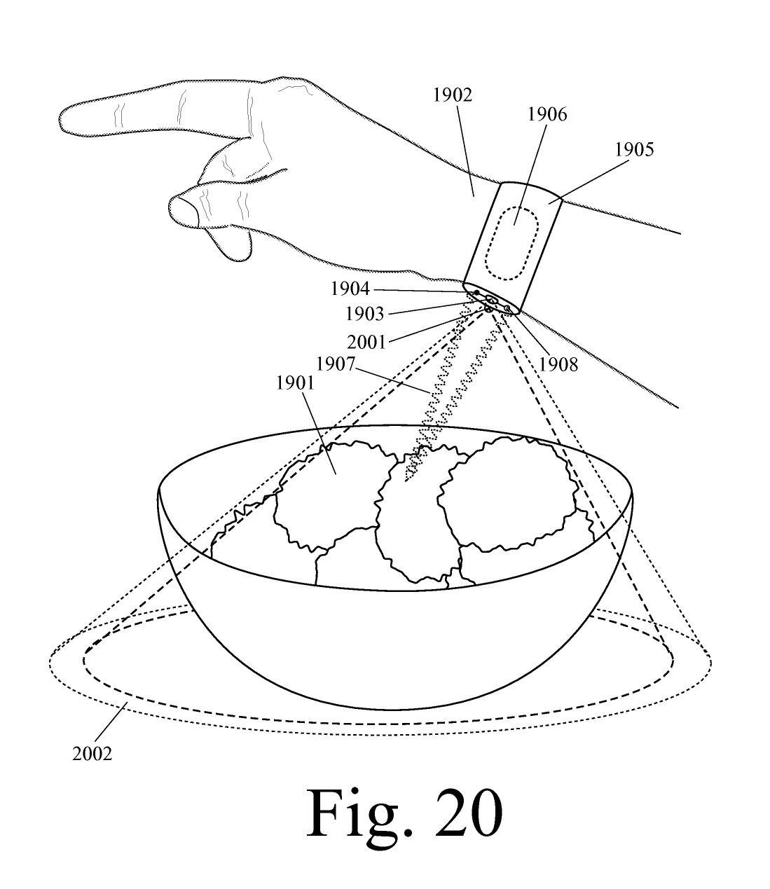

FIG. 20 shows an example of how a device can be embodied in a wearable device for food identification and quantification which is the same as the embodiment shown in FIG. 19, except that FIG. 20 further comprises a light-emitting member 2001 which projects a light-based fiducial marker 2002 on, or in proximity to, nearby food 1901 to better estimate the size of food 1901. In an example, light-emitting member 2001 can be a laser which emits coherent light.

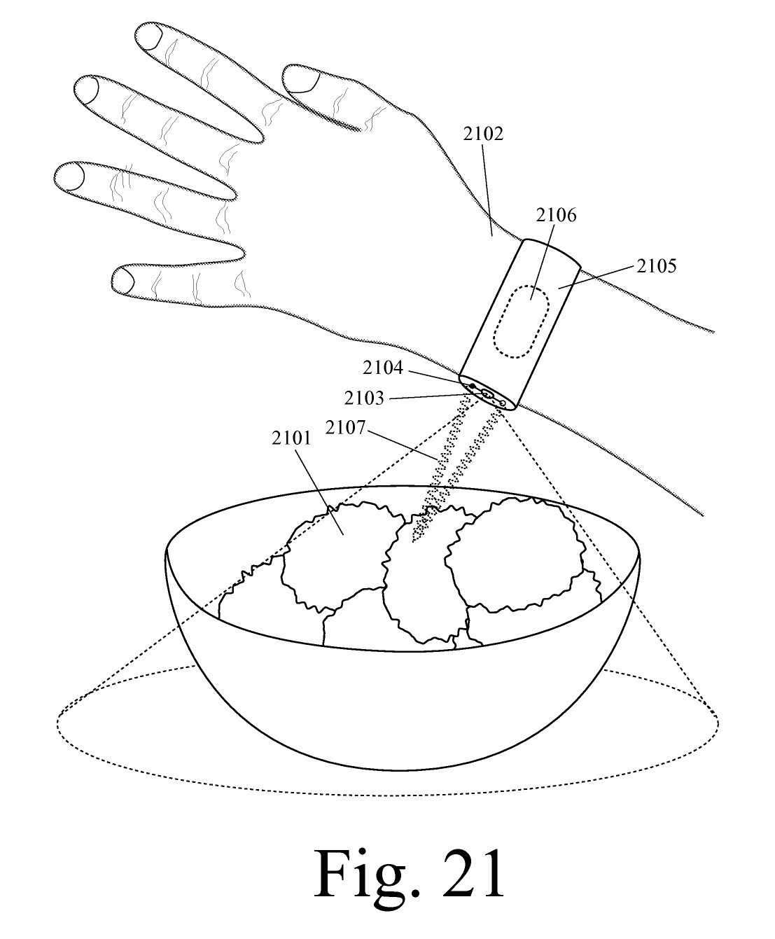

FIG. 21 shows an example which is similar to that shown in FIG. 21 except that the attachment mechanism in FIG. 21 holds the imaging member and the optical sensor on a lateral/narrow side of a person's wrist. FIG. 21 shows an example of how a device can be embodied in a wearable device for food identification and quantification comprising: at least one imaging member 2103, wherein this imaging member takes pictures and/or records images of nearby food 2101, and wherein these food pictures and/or images are automatically analyzed to identify the types and quantities of food; an optical sensor 2104, wherein this optical sensor collects data concerning light 2107 that is transmitted through or reflected from nearby food 2101, and wherein this data is automatically analyzed to identify the types of food 2101, the types of ingredients in food 2101, and/or the types of nutrients in food 2101; one or more attachment mechanisms 2105, wherein these one or more attachment mechanisms are configured to hold the imaging member 2103 and the optical sensor 2104 in close proximity to the surface of a person's body 2102; and an image-analyzing member 2106 which automatically analyzes food pictures and/or images. In an example, there can be two or more imaging members. In an example, there can be two imaging members, one on each of the two opposite lateral/narrow sides of a person's wrist.

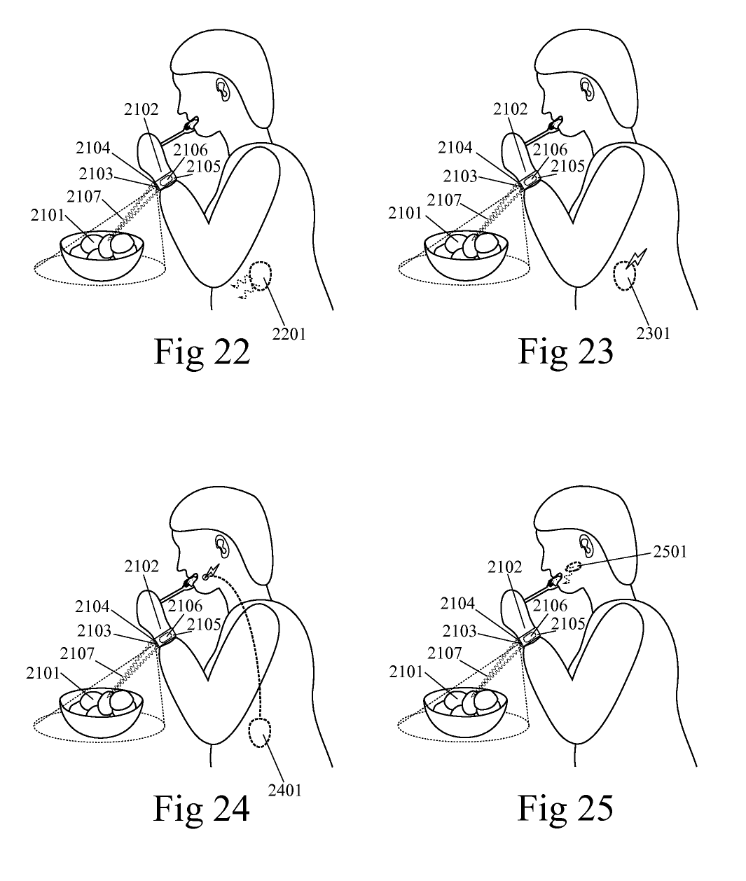

### Narrative to Accompany FIGS. 22 Through 28:

FIGS. 22 through 28 show examples of how a device can be embodied in a wearable system or device for food identification and nutritional intake modification comprising: at least one imaging member, wherein this imaging member takes pictures and/or records images of nearby food, and wherein these food pictures and/or images are automatically analyzed to identify the types and quantities of food; an optical sensor, wherein this optical sensor collects data concerning light that is transmitted through or reflected from nearby food, and wherein this data is automatically analyzed to identify the types of food, the types of ingredients in the food, and/or the types of nutrients in the food; one or more attachment mechanisms, wherein these one or more attachment mechanisms are configured to hold the imaging member and the optical sensor in close proximity to the surface of a person's body; an image-analyzing member which automatically analyzes food pictures and/or images; and a computer-to-human interface which modifies the person's nutritional intake. The examples shown in FIGS. 22 through 28 can further comprise any of the variations in components or methods which were discussed herein in other sections.

FIG. 22 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and computer-to-human interface 2201 which modifies the person's nutritional intake. As discussed earlier, unhealthy types and/or quantities of food, ingredients, or nutrients can be identified based on data from the imaging member and the optical sensor.

In this example, computer-to-human interface 2201 is an implanted substance-releasing device. In this example, computer-to-human interface 2201 allows normal absorption of nutrients from healthy types and/or quantities of food, but reduces absorption of nutrients from unhealthy types and/or quantities of food. In this example, computer-to-human interface 2201 reduces consumption and/or absorption of nutrients from unhealthy types and/or quantities of food by releasing an absorption-reducing substance into the person's gastrointestinal tract. In this example, computer-to-human interface 2201 releases an absorption-reducing substance into the person's stomach.

FIG. 23 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and computer-to-human interface 2301 which modifies the person's nutritional intake. As discussed earlier, unhealthy types and/or quantities of food, ingredients, or nutrients can be identified based on information from the imaging member and the optical sensor.

In this example, computer-to-human interface 2301 is an implanted electromagnetic energy emitter. In this example, computer-to-human interface 2301 allows normal absorption of nutrients from healthy types and/or quantities of food, but reduces absorption of nutrients from unhealthy types and/or quantities of food. In this example, computer-to-human interface 2301 reduces consumption and/or absorption of nutrients from unhealthy types and/or quantities of food by delivering electromagnetic energy to a portion of the person's gastrointestinal tract and/or to nerves which innervate that portion. In this example, computer-to-human interface 2301 delivers electromagnetic energy to the person's stomach and/or to a nerve which innervates the stomach.

FIG. 24 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and computer-to-human interface 2401 which modifies the person's nutritional intake. As discussed earlier, unhealthy types and/or quantities of food, ingredients, or nutrients can be identified based on information from the imaging member and the optical sensor.

In this example, computer-to-human interface 2401 is an implanted electromagnetic energy emitter. In this example, computer-to-human interface 2401 allows normal consumption (and/or absorption) of nutrients from healthy types and/or quantities of food, but reduces consumption (and/or absorption) of nutrients from unhealthy types and/or quantities of food. In this example, computer-to-human interface 2401 reduces consumption and/or absorption of nutrients from unhealthy types and/or quantities of food by delivering electromagnetic energy to nerves which innervate a person's tongue and/or nasal passages. In an example, this electromagnetic energy can reduce taste and/or smell sensations. In an example, this electromagnetic energy can create virtual taste and/or smell sensations.

FIG. 25 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and computer-to-human interface 2501 which modifies the person's nutritional intake. As discussed earlier, unhealthy types and/or quantities of food, ingredients, or nutrients can be identified based on information from the imaging member and the optical sensor.

In this example, computer-to-human interface 2501 is an implanted substance-releasing device. In this example, computer-to-human interface 2501 allows normal consumption (and/or absorption) of nutrients from healthy types and/or quantities of food, but reduces consumption (and/or absorption) of nutrients from unhealthy types and/or quantities of food. In this example, computer-to-human interface 2501 reduces consumption and/or absorption of nutrients from unhealthy types and/or quantities of food by releasing a taste and/or smell modifying substance into a person's oral cavity and/or nasal passages. In an example, this substance can overpower the taste and/or smell of food. In an example, this substance can be released selectively to make unhealthy food taste or smell bad.

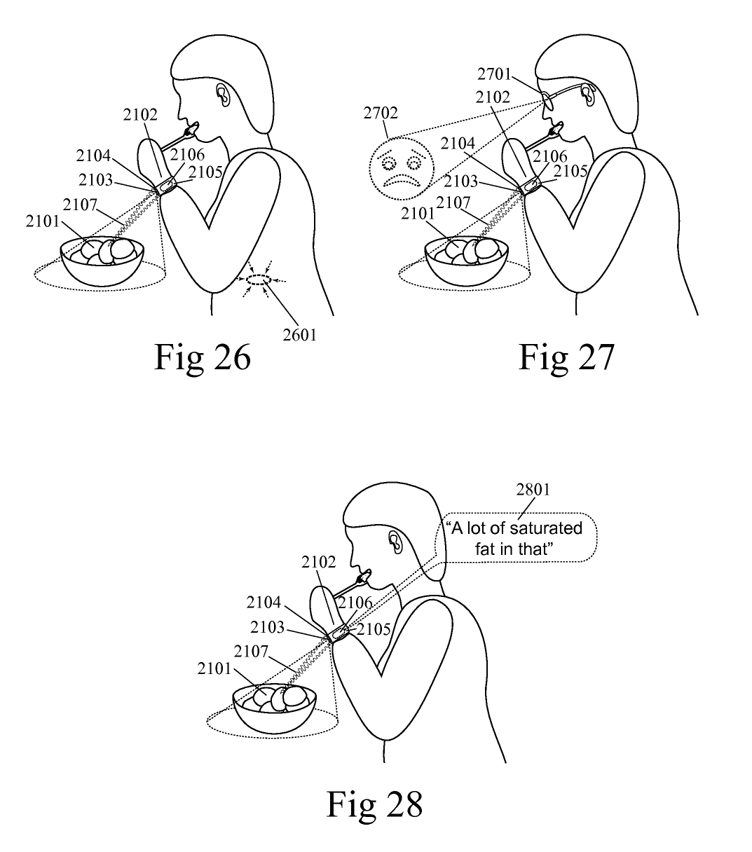

FIG. 26 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and computer-to-human interface 2601 which modifies the person's nutritional intake. As discussed earlier, unhealthy types and/or quantities of food, ingredients, or nutrients can be identified based on information from the imaging member and the optical sensor.

In this example, computer-to-human interface 2601 is an implanted gastrointestinal constriction device. In this example, computer-to-human interface 2601 allows normal consumption (and/or absorption) of nutrients from healthy types and/or quantities of food, but reduces consumption (and/or absorption) of nutrients from unhealthy types and/or quantities of food. In this example, computer-to-human interface 2601 reduces consumption and/or absorption of nutrients from unhealthy types and/or quantities of food by constricting, slowing, and/or reducing passage of food through the person's gastrointestinal tract. In an example, this computer-to-human interface 2601 is a remotely-adjustable gastric band.

FIG. 27 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and a computer-to-human interface (comprising eyewear 2701 and virtual image 2702) which modifies the person's nutritional intake. As discussed earlier, unhealthy types and/or quantities of food, ingredients, or nutrients can be identified based on information from the imaging member and the optical sensor.

In this example, the computer-to-human interface comprises eyewear 2701 (with which image-analyzing member 2106 is in wireless communication) and a virtually-displayed image 2702. In this example, virtually-displayed image 2702 is a frowning face which is shown in proximity to unhealthy food 2101. In an example, a virtually-displayed image or food information can be shown in a person's field of vision as part of augmented reality. In an example, a virtually-displayed image or food information can be shown on the surface of a wearable or mobile device. In this example, this computer-to-human interface allows normal consumption of nutrients from healthy types and/or quantities of food, but discourages consumption of nutrients from unhealthy types and/or quantities of food. In this example, a computer-to-human interface discourages consumption and/or absorption of nutrients from unhealthy types and/or quantities of food by displaying negative images or other visual information in a person's field of view. In this example, a computer-to-human interface provides negative stimuli in association with unhealthy types and quantities of food and/or provides positive stimuli in association with healthy types and quantities of food. This example can include other types of informational displays and other component variations which were discussed earlier.

FIG. 28 shows an example of how a device can be embodied in a wearable system or device for food identification and nutritional intake modification comprising: imaging member 2103, wherein imaging member 2103 takes pictures and/or records images of nearby food 2101, and wherein these food pictures and/or images are automatically analyzed to identify the types and quantities of food 2101; optical sensor 2104, wherein optical sensor 2104 collects data concerning light 2107 that is reflected from nearby food 2101, and wherein this data is automatically analyzed to identify the types of food 2101, the types of ingredients in food 2101, and/or the types of nutrients in food 2101; attachment mechanism 2105, wherein attachment mechanism 2105 is configured to hold imaging member 2103 and optical sensor 2104 in close proximity to the surface of a person's body 2102; image-analyzing member 2106 which automatically analyzes food pictures and/or images; and a computer-to-human interface which modifies the person's nutritional intake. As discussed earlier, unhealthy types and/or quantities of food, ingredients, or nutrients can be identified based on information from the imaging member and the optical sensor.

In this example, the computer-to-human interface comprises an audio message 2801 which is communicated to the person wearing the device. In an example, this audio message can be emitted from a speaker or other sound-emitting component which is incorporated into attachment mechanism 2105. In this example, the computer-to-human interface allows normal consumption of nutrients from healthy types and/or quantities of food, but discourages consumption of nutrients from unhealthy types and/or quantities of food. In this example, the computer-to-human interface discourages consumption and/or absorption of nutrients from unhealthy types and/or quantities of food by sending an audio communication to the person wearing the imaging member and/or to another person. In this example, a computer-to-human interface provides negative stimuli in association with unhealthy types and quantities of food and/or provides positive stimuli in association with healthy types and quantities of food. This example can include other types of computer-to-human communication and other component variations which were discussed earlier.

A device can be embodied as a wearable device or system for identification and quantification of food, ingredients, and/or nutrients. In an example, a device can comprise: (a) at least one imaging member (such as a camera) that takes pictures of nearby food, wherein these food pictures are automatically analyzed to identify the types and quantities of food, ingredients, and/or nutrients; (b) an optical sensor (such as a spectroscopic optical sensor) which collects data concerning light that is reflected from nearby food, wherein this data is automatically analyzed to identify types of food, ingredients in the food, and/or nutrients in the food; (c) an attachment mechanism (such as a wrist band) which holds the imaging member and the optical sensor in close proximity to the surface of a person's body; and (d) an image-analyzing member (such as a data control unit).

In an example, a device can further comprise a computer-to-human interface which modifies a person's food consumption and/or nutritional intake based on identification of unhealthy vs. healthy types and quantities of food, ingredients, and/or nutrients. In an example, a device can encourage consumption and/or increase nutritional intake of healthy food, ingredients, and/or nutrients and can discourage consumption and/or decrease nutritional intake of unhealthy food, ingredients, and/or nutrients.

In an example, a device can serve as the energy-input measuring component of an overall system for energy balance and weight management. In an example, information from a device can be combined with information from a separate caloric expenditure monitoring device in order to comprise an overall system for energy balance, fitness, weight management, and health improvement. This device is not a panacea for good nutrition, energy balance, and weight management, but it can be a useful part of an overall strategy for encouraging good nutrition, energy balance, weight management, and health improvement.

In an example, a wearable device or system for food identification and quantification can comprise: (a) at least one imaging member, wherein this imaging member takes pictures and/or records images of nearby food, and wherein these food pictures and/or images are automatically analyzed to identify the types and quantities of food; (b) an optical sensor, wherein this optical sensor collects data concerning light that is transmitted through or reflected from nearby food, and wherein this data is automatically analyzed to identify the types of food, the types of ingredients in the food, and/or the types of nutrients in the food; (c) one or more attachment mechanisms, wherein these one or more attachment mechanisms are configured to hold the imaging member and the optical sensor in close proximity to the surface of a person's body; and (d) an image-analyzing member which automatically analyzes food pictures and/or images.

In an example, the at least one imaging member can be a camera. In an example, an imaging member can be configured to have a focal direction which points outward from the surface of a person's body or clothing. In an example, an optical sensor can be a spectroscopic optical sensor that collects data concerning the spectrum of light that is transmitted through and/or reflected from nearby food. In an example, an optical sensor can be configured to have a sensing direction which points outward from the surface of a person's body or clothing. In an example, an attachment mechanism can be selected from the group consisting of: arm band, bracelet, brooch, collar, cuff link, dog tags, ear ring, ear-mounted bluetooth device, eyeglasses, finger ring, headband, hearing aid, necklace, pendant, wearable mouth microphone, wrist band, and wrist watch. In an example, an image-analyzing member can be a data control unit.

In an example, close proximity can be defined as being less than three inches away. In an example, an attachment mechanism can be configured to hold at least one imaging member in close proximity to a person's wrist, finger, hand, and/or arm. In an example, an attachment mechanism can comprise a wrist band, bracelet, and/or smart watch which is configured to hold at least one imaging member on a person's wrist. In an example, an attachment mechanism can comprise a wrist band, bracelet, and/or smart watch which is configured to hold at least one imaging member on the anterior/palmar/lower side or a lateral/narrow side of a person's wrist for imaging nearby food.

In an example, an attachment mechanism can be configured to hold at least one imaging member in close proximity to a person's neck or head. In an example, an attachment mechanism can comprise a neck-encircling member which is configured to hold at least one imaging member in proximity to a person's neck. In an example, an attachment mechanism can comprise eyewear which is configured to hold at least one imaging member in close proximity to a person's head. In an example, an attachment mechanism can be configured to hold an optical sensor in close proximity to a person's wrist, finger, hand, and/or arm. In an example, an attachment mechanism can comprise a wrist band, bracelet, and/or smart watch which is configured to hold an optical sensor on a person's wrist.

In an example, an attachment mechanism can comprise a wrist band, bracelet, and/or smart watch which is configured to hold an optical sensor on the anterior/palmar/lower side or a lateral/narrow side of a person's wrist for scanning nearby food. In an example, a light-emitting member can project a light-based fiducial marker on, or in proximity to, nearby food to estimate food size.

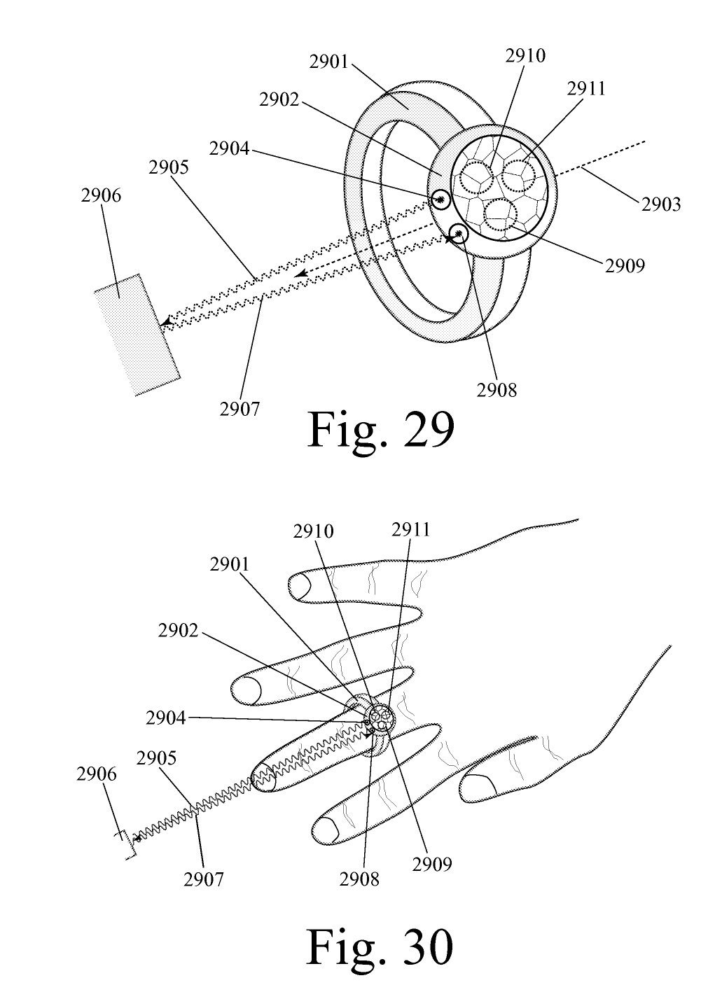

### Narrative to Accompany FIGS. 29 and 30:

In an example, an optical sensor can be configured to have a sensing direction which points outward from the surface of a person's body or clothing. In an example, an optical sensor can be a spectroscopic optical sensor. In an example, a spectroscopic sensor can be a part of a wearable device which is configured to be worn on a person's finger. In an example, a spectroscopic sensor can be a part of an electronically-functional ring. A wearable sensor can be worn on a person in a manner like a finger ring. In an example, a spectroscopic sensor can collect data concerning the spectrum of light that is transmitted through and/or reflected from nearby food. In an example, a sensor can be selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

FIGS. 29 and 30 show an example of a spectroscopic finger ring for compositional analysis of food or some other environmental object. This spectroscopic finger ring is one embodiment of a wearable device configured worn on a person's hand including a spectroscopic optical sensor that collects data concerning the spectrum of light that is reflected from (or has passed through) nearby food or some other environmental object. This light spectrum data is analyzed in order to estimate the chemical composition of the food or other environmental object. FIG. 29 shows a close-up view of this finger ring before it is worn. FIG. 30 shows an overall view of this same finger as it is worn on a person's hand.

The example shown in FIGS. 29 and 30 is a spectroscopic finger ring for compositional analysis of environmental objects comprising: a ring which is configured to be worn on a person's finger, wherein this ring further comprises a light-emitting member which projects a beam of light away from the person's body toward food or some other environmental object, and wherein this ring further comprises a spectroscopic optical sensor which collects data concerning the spectrum of light which is reflected from (or has passed through) the food or other environmental object.

Looking at this example in more detail, FIGS. 29 and 30 show: a finger-encircling portion 2901 of a finger ring; an anterior (or upper) portion 2902 of the finger ring; a central proximal-to-distal axis 2903 of the finger ring; a light-emitting member 2904; an outward-directed light beam 2905; a piece of food or other environmental object 2906; an inward-directed light beam 2907; a spectroscopic optical sensor 2908; a data processing unit 2909; a power source 2910; and a data transmitting unit 2911.

In an example, a finger-encircling portion of a ring can have a shape which is selected from the group consisting of: circle, ellipse, oval, cylinder, torus, and volume formed by three-dimensional revolution of a semi-circle. In an example, a finger-encircling portion of a ring can be made from a metal or polymer. In an example, a finger-encircling portion of a ring can have a proximal-to-distal width between ⅛″ to 2″. In an example, proximal can be defined as closer to a person's elbow (or further from a finger tip) and distal can be defined as further from a person's elbow (or closer to a finger tip).

In an example, an anterior (or upper) portion of a finger ring can be made separately and then attached to the finger-encircling portion of the ring. In an example, an anterior (or upper) portion of a finger ring can be an integral portion of the finger-encircling portion of the ring which widens, thickens, bulges, spreads, and/or bifurcates as it spans the anterior (or upper) surface of a finger. In an example, an anterior (or upper) portion of a finger ring can have a cross-sectional shape which is selected from the group consisting of: circle, ellipse, oval, egg shape, tear drop, hexagon, octagon, quadrilateral, and rounded quadrilateral. In an example, an anterior (or upper) portion of a finger ring can be ornamental. In an example, an anterior (or upper) portion of a finger ring can be a gemstone or at least look like a gemstone. In an example, an anterior (or upper) portion of a finger ring can include a display screen. In an example, the anterior (or upper) portion of a finger ring can rotate.

In an example, a central proximal-to-distal axis of a finger ring can be defined as the straight line which most closely fits a proximal-to-distal series of centroids of interior cross-sectional perimeters of the finger-encircling portion of the finger ring. If the shape of a finger ring is approximated by a cylinder or torus, then its central proximal-to-distal axis connects the centers of cross-sectional circles comprising the cylinder or torus. In an example, a finger proximal-to-distal axis can be defined as the central longitudinal axis of a phalange on which a finger ring is configured to be worn. If the shape of a phalange is approximated by a cylinder, then its central proximal-to-distal axis connects the centers of cross-sectional circles comprising the cylinder.

In an example, a light-emitting member can be an LED (Light Emitting Diode). In an example, a light-emitting member can be a laser. In an example, a spectroscopic finger ring can have two or more light-emitting members instead of just one. In an example, a light-emitting member can emit an outward-directed beam of light away from the surface of a person's body. In an example, an outward-directed beam of light from a light-emitting member can comprise near-infrared light. In an example, an outward-directed beam of light from a light-emitting member can comprise infrared light. In an example, an outward-directed beam of light from a light-emitting member can comprise ultra-violet light. In an example, an outward-directed beam of light from a light-emitting member can comprise white light. In an example, an outward-direction beam of light from a light-emitting member can comprise coherent light. In an example, an outward-direction beam of light from a light-emitting member can comprise polarized light.

In an example, a light-emitting member can be part of (or attached to) the anterior (or upper) portion of a finger ring. In an example, a spectroscopic optical sensor in a finger ring can have an outward projection vector which points away from a person's body and toward food or some other environmental object. In an example, a light-emitting member can emit an outward-directed beam of light from the distal portion of the anterior (or upper) portion of a finger ring. In an example, a light-emitting member can emit an outward-directed beam of light in a proximal-to-distal direction. In an example, when a person points their finger at food or some other environmental object, then this outward-directed beam is directed toward that food or other environmental object. In an example, when a person grasps food or some other environmental object with their hand, then this outward-directed beam is directed toward that food or other environmental object.

In an example, a light-emitting member can emit an outward-directed beam of light in a proximal-to-distal vector which is substantially parallel to the central proximal-to-distal axis of a finger ring. In an example, a light-emitting member can emit an outward-directed beam of light in a proximal-to-distal vector which is substantially parallel to the proximal-to-distal axis of the phalange on which a ring is worn. In an example, a light-emitting member can emit an outward-directed beam of light along a vector which intersects (or whose virtual forward or backward extension intersects) a line which is parallel to the central proximal-to-distal axis of the finger ring. In an example, this intersection forms a distal-opening (or proximal-pointing) angle theta. In an example, the absolute value of theta is less than 20 degrees. In an example, the absolute value of theta is less than 45 degrees. In an example, a light-emitting member can emit an outward-directed beam of light along a vector which intersects (or whose virtual forward or backward extension intersects) a line which is parallel to the central proximal-to-distal axis of the phalange on which the ring is worn. In an example, this intersection forms a distal-opening (or proximal-pointing) angle theta. In an example, the absolute value of theta is less than 20 degrees. In an example, the absolute value of theta is less than 45 degrees.

In an example, the vector direction of an outward-directed beam of light emitted by a light-emitting member can be changed by the person wearing the finger ring. In an example, this vector can be automatically changed by the device in response to (changes in) the location of food or some other environmental object. In an example, this vector can be automatically moved in an iterative manner in order to automatically scan for food or some other environmental object. In an example, this vector can be automatically moved in an iterative manner in order to automatically scan a large portion of the surface of food or some other environmental object. In an example, the vector direction of an outward-directed beam of light can be changed by rotating the anterior (or upper) portion of a finger ring. In an example, the vector direction of an outward-directed beam of light can be changed by moving a mirror inside the anterior (or upper) portion of a finger ring.

In an example, a spectroscopic optical sensor can receive inward-directed light which has been reflected from (or passed through) food or some other environmental object. In an example, the reflection of light from the surface of the food or some other environmental object changes the spectrum of light which is then measured by the spectroscopic optical sensor in order to estimate the chemical composition of the food or other environmental object. In an example, the passing of light through food or some other environmental object changes the spectrum of light which is then measured by the spectroscopic optical sensor in order to estimate the chemical composition of the food or other environmental object. In an example, inward-directed light can originate with the outward-directed beam of light from the light-emitting member. In an example, inward-directed light can originate from an ambient light source.

In an example, data from a spectroscopic optical sensor can be analyzed in order to estimate the chemical composition of food or some other environmental object. In an example, data from a spectroscopic optical sensor can be analyzed in order to measure the composition of an environmental object from which an outward-directed beam of light has been reflected. In an example, a spectroscopic optical sensor can be selected from the group consisting of: spectrometry sensor; white light and/or ambient light spectroscopic sensor; infrared spectroscopic sensor; near-infrared spectroscopic sensor; ultraviolet spectroscopic sensor; ion mobility spectroscopic sensor; mass spectrometry sensor; backscattering spectrometric sensor; and spectrophotometer.

In an example, a light-emitting member and a spectroscopic optical sensor can share the same opening, compartment, or location in a finger ring. In an example, a light-emitting member and a spectroscopic optical sensor can be aligned along the same proximal-to-distal axis. In an example, an outward-directed beam of light emitted by a light-emitting member can be substantially parallel to (and even coaxial with) an inward-directed beam of light received by a spectroscopic optical sensor. In an example, a light-emitting member and a spectroscopic optical sensor can occupy different openings, compartments, or locations on a finger ring. In an example, an outward-directed beam of light emitted by a light-emitting member and an inward-directed beam of light received by a spectroscopic optical sensor can travel at different angles along non-parallel vectors.

In an example, the vector along which an outward-directed beam of light is emitted can be selected in order to direct reflected light back to the spectroscopic optical sensor from an object at a selected focal distance. In an example, this selected focal distance can be selected manually by the person wearing the ring. In an example, this selected focal distance can be selected based on detection of food or some other environmental object at a selected distance from the ring. In an example, detection of food or some other environmental object (and its distance) can be based on image analysis, reflection of light energy, reflection of radio waves, reflection of sonic energy, or gesture recognition. In an example, the vector along which an outward-directed beam of light is emitted can be varied in order to scan across different distances (or focal depths) in the surrounding environment.

In an alternative example, a spectroscopic finger ring can have an optical spectroscopic sensor, but no light-emitting member. In such an example, an optical spectroscopic sensor can receive ambient light which has been reflected from (or passed through) food or some other environmental object. In an alternative example, a spectroscopic finger ring can have a member which reflects and/or redirects ambient light toward food or some other environmental object instead of using a light-emitting member. In such an example, a spectroscopic finger ring can have a mirror or lens which is adjusted in order to direct sunlight (or other ambient light) toward food or some other environmental object. In an example, the reflection of this ambient light from the food or other environmental object can be analyzed in order to estimate the chemical composition of the food or other environmental object.

In an example, a finger ring device can further comprise a motion sensor. In an example, a finger ring device can further comprise an accelerometer and/or gyroscope. In an example, motion patterns can be analyzed to determine optimal times for initiating a spectroscopic scan of food or some other environmental object. In an example, motion patterns can be analyzed to identify eating patterns. In an example, spectroscopic scans can be triggered at times during eating when a person's arm is most extended and, thus, most likely to be closest to the remaining uneaten portion of food. In an example, a spectroscopic scan can be triggered by a gesture indicating that a person is grasping food or bringing food up to their mouth. In an example, repeated spectroscopic scans of food at different times during a meal can help to analyze the composition of multiple food layers, not just the surface layer. This can provide a more accurate estimate of food composition, especially for foods with different internal layers and/or a composite (non-uniform) ingredient structure.

In an example, a finger ring device can further comprise a visible laser beam. In an example, this visible laser beam can be separate from the outward-directed beam of light that is used for spectroscopic analysis. In an example, a visible laser beam can be used by the person in order to point the spectroscopic beam toward food or some other environmental object for compositional analysis. In an example, a person can “point and click” by pointing the laser beam toward an object and then tapping, clicking, or pressing a portion of the finger ring in order to initiate a spectroscopic scan of the object. In an example, a person can point the laser beam toward the object and then give a verbal command to initiate a spectroscopic scan of the object. In an example, a finger ring device can further comprise a camera which takes a picture of the food or other environmental object. In an example, spectroscopic analysis can reveal the composition of the food (or object) and analysis of images from the camera can estimate the size of the food (or object). In an example, a visible laser beam can serve as a fiducial marker for image analysis.

In an example, a spectroscopic finger ring can be controlled by gesture recognition. In an example, a spectroscopic finger ring can be triggered by pointing at food or some other environmental object. In an example, a spectroscopic finger ring can be controlled by making a specific hand gesture. In an example, a spectroscopic finger ring can be directed to scan the entire surface of nearby food or some other environmental object by a hand gesture.

In an example, a spectroscopic finger ring can be worn on the proximal phalange of a person's finger, in a manner like a conventional ring. In an example, a spectroscopic finger ring can be worn on the middle or distal phalange of a person's finger in order to be more accurately directed toward an object held between the fingers, grasped by the hand, or pointed at by the person. In an example, a spectroscopic finger ring can be worn on a person's ring finger, in a manner like a conventional ring. In an example, a spectroscopic finger ring can be worn on a person's index finger in order to be more accurately directed toward an object held between the person's fingers, grasped by the person's hand, or pointed at by the person. In an example, a spectroscopic finger ring can be worn on a person's middle finger or pinky. In an example, joint analysis of data from a plurality of spectroscopic finger rings can provide more accurate information than data from a single spectroscopic finger ring. In an example, a plurality of spectroscopic finger rings can be worn on the proximal, middle, and/or distal phalanges of a person's finger. In an example, a plurality of spectroscopic finger rings can be worn on a person's index, middle, ring, and/or pinky fingers.

In an example, a finger ring device can further comprise a local data processing unit. In an example, data from an optical spectroscopic sensor can be at least partially processed by this local data processing unit. In an example, this data can be wirelessly transmitted to a remote data processing unit for further processing. In an example, this finger ring device can further comprise a data transmitting unit which wirelessly transmits data to another device and/or system component. In an example, the spectrum of light which has been reflected from (or passed through) food or some other environmental object can be used to help identify the chemical composition of that food or other environmental object. In an example, a change in the spectrum of outward-directed light from a light-emitting member vs. the spectrum of inward-directed light which has been reflected from (or passed through) food or some other environmental object can be used to help identify the chemical composition of that food or other environmental object.

In an example, a spectroscopic finger ring can be in wireless electromagnetic communication with a remote device. In an example, this remote device can be worn elsewhere on the person's body. In an example, a spectroscopic finger ring can be in electromagnetic communication with a smart watch or other wrist-worn device. In an example, information concerning the chemical composition of food or some other environmental object can be displayed on a smart watch or other wrist-worn device. In an example, a spectroscopic finger ring can be in electromagnetic communication with electronically-functional and/or augmented reality eyewear. In an example, information concerning the chemical composition of food or some other environmental object can be displayed via electronically-functional and/or augmented reality eyewear. In an example, a spectroscopic finger ring can be in wireless electromagnetic communication with a hand held device such as a cell phone. In an example, information concerning the chemical composition of food or some other environmental object can be displayed on a cell phone or other hand held electronic device.

In an example, information concerning the composition of food or some other environmental object based on data from a spectroscopic finger ring can be communicated in an auditory manner. In an example, this information can be communicated by voice from a wrist-worn device, electronically-functional eyewear, electronically-functional earwear, or a hand-held electronic device. For example, a person can point at an energy bar which is labeled “100% natural” and electronically-functional earwear can whisper into the person's ear—“Yeah, right . . . 50% natural sugar, 40% natural corn syrup, and 10% natural caffeine. They can call it natural, but it is not good nutrition.”

In an example, this finger ring device can further comprise a power source such as a battery and/or and energy-harvesting unit. In an example, an energy-harvesting unit can harvest energy from body motion, body temperature, ambient light, and/or ambient electromagnetic energy. In various examples, other relevant components and features discussed with respect to other examples in this disclosure can also be applied to the example shown in FIGS. 29 and 30.

### Concluding Examples Based on FIGS. 1 Through 30:

In various examples, FIGS. 1 through 30 show how this invention can be embodied in a wearable device for food identification and quantification comprising: (a) a camera which takes pictures of nearby food, wherein these food pictures are analyzed in order to identify the types and quantities of food; (b) a light-emitting member which projects a light-based fiducial marker on, or in proximity to, the nearby food as an aid in estimating food size; (c) a spectroscopic optical sensor, wherein this spectroscopic optical sensor collects data concerning light that is reflected from, or has passed, through the nearby food and wherein this data is analyzed to identify the types of food, the types of ingredients in the food, and/or the types of nutrients in the food; (d) an attachment mechanism, wherein this attachment mechanism is configured to hold the camera, the light-emitting member, and the spectroscopic optical sensor in close proximity to the surface of a person's body; and (e) an image-analyzing member which analyzes the food pictures.

In an example, an attachment mechanism can be configured to be worn on or around a person's finger. In an example, an attachment mechanism can be configured to be worn on or around a person's wrist and/or forearm. In an example, an attachment mechanism can be configured to be worn on, in, or around a person's ear. In an example, an attachment mechanism can be configured to be worn on or over a person's eyes. In an example, an attachment mechanism can be configured to be worn on or around a person's neck.

In various examples, FIGS. 1 through 30 also show how this invention can be embodied in a wearable spectroscopic device for compositional analysis of environmental objects comprising: a finger ring, wherein this finger ring further comprises: (a) a finger-encircling portion, wherein this finger-encircling portion is configured to encircle at least 70% of the circumference of a person's finger, wherein this finger-encircling portion has an interior surface which is configured to face toward the surface of the person's finger when worn, wherein this finger-encircling portion has a central proximal-to-distal axis which is defined as the straight line which most closely fits a proximal-to-distal series of centroids of cross-sections of the interior surface, and wherein proximal is defined as being closer to a person's elbow and distal is defined as being further from a person's elbow when the person's arm, hand, and fingers are fully extended; (b) a light-emitting member which projects a beam of light along a proximal-to-distal vector toward an object in the person's environment, wherein this vector, or a virtual extension of this vector, is either parallel to the central proximal-to-distal axis or intersects a line which is parallel to the central proximal-to-distal axis forming a distally-opening angle whose absolute value is less than 45 degrees; and (c) a spectroscopic optical sensor which collects data concerning the spectrum of light which is reflected from, or has passed through, the object in the person's environment, wherein data from the spectroscopic optical sensor is used to analyze the composition of this object, and wherein this spectroscopic optic sensor is selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

In an example, a beam of light projected by a light-emitting member can be near-infrared light, infrared light, or ultra-violet light. In an example, a beam of light projected by a light-emitting member can be white light and/or reflected ambient light. In an example, a beam of light projected by a light-emitting member can be coherent light. In an example, this device can further comprise a laser pointer which is moved by the person in order to direct a visible beam of coherent light toward an object in the environment in order to guide, direct, select, adjust, and/or trigger spectroscopic analysis of this object.

In an example, the vector of a beam of light projected by a light-emitting member can be automatically changed in response to detection of an object in the environment and/or changes in the location of an object in the environment. In an example, the vector of a beam of light projected by a light-emitting member can be selected in order to direct reflected light back to a spectroscopic optical sensor from an object at a selected focal distance, wherein this selected focal distance can be selected based on detection of the object at the selected distance, and wherein measurement of the object's distance can be based on image analysis, reflection of light energy, reflection of radio waves, reflection of sonic energy, and/or gesture recognition. In an example, the vector of a beam of light emitted by a light-emitting member can be varied in order to scan for objects in the environment at different distances and/or to scan a larger portion of the surface of an object in the environment.

In an example, this device can further comprise a data processing unit which at least partially processes data from the spectroscopic optical sensor. In an example, this device can further comprise a wireless data transmitter through which the device is in wireless communication with another wearable device and/or a remote computer and wherein information concerning the composition of an environmental object is displayed by the other wearable device and/or remote computer.

In an example, this device can further comprise a motion sensor. Motion patterns can be analyzed in order to trigger or adjust the parameters of a spectroscopic scan of an object in the environment. In an example, a spectroscopic scan can be triggered when motion patterns indicate that a person is eating. In an example, a device can perform multiple spectroscopic scans, at different times, while a person is eating in order to better analyze the overall composition of food with different internal layers and/or a non-uniform ingredient structure.

In various examples, FIGS. 1 through 30 show how this invention can be embodied in a wearable spectroscopic device for compositional analysis of environmental objects comprising: a finger ring, wherein this finger ring further comprises: (a) a finger-encircling portion, wherein this finger-encircling portion is configured to encircle at least 70% of the circumference of a person's finger when worn, wherein a virtual cylinder is defined as the cylinder which most closely approximates the shape of the finger-encircling portion, wherein this finger-encircling portion has a central proximal-to-distal axis which is defined as the central longitudinal axis of the virtual cylinder; (b) a light-emitting member, wherein this light-emitting member projects a beam of light toward an object in the person's environment, and wherein this vector, or a virtual-extension of this vector, is either parallel to the central proximal-to-distal axis or intersects a line which is parallel to the central proximal-to-distal axis forming a distally-opening angle whose absolute value is less than 45 degrees; and (c) a spectroscopic optical sensor, wherein this spectroscopic optical sensor which collects data concerning the spectrum of light which is reflected from or has passed through the object in the person's environment, wherein data from the spectroscopic optical sensor is used to analyze the composition of this object, and wherein this spectroscopic optic sensor is selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer; and (d) a laser pointer, wherein this laser pointer projects a visible beam of coherent light toward an object in the person's environment, and wherein this beam of coherent light is used by the person to select this object for spectroscopic analysis.

I claim:

1. A wearable device for food identification and quantification comprising:
a camera which takes pictures of nearby food, wherein these food pictures are analyzed in order to identify the types and quantities of food;
a light-emitting member which projects a light-based fiducial marker on, or in proximity to, the nearby food as an aid in estimating food size;
a spectroscopic optical sensor, wherein this spectroscopic optical sensor collects data concerning light that is reflected from, or has passed through, the nearby food and wherein this data is analyzed to identify the types of food, the types of ingredients in the food, and/or the types of nutrients in the food;
an attachment mechanism, wherein this attachment mechanism is configured to hold the camera, the light-emitting member, and the spectroscopic optical sensor in close proximity to the surface of a person's body; and
an image-analyzing member which analyzes the food pictures.

2. The device in claim 1 wherein the attachment mechanism is configured to be worn on or around the person's finger.

3. The device in claim 1 wherein the attachment mechanism is configured to be worn on or around the person's wrist and/or forearm.

4. The device in claim 1 wherein the attachment mechanism is configured to be worn on, in, or around the person's ear.

5. The device in claim 1 wherein the attachment mechanism is configured to be worn on or over the person's eyes.

6. The device in claim 1 wherein the attachment mechanism is configured to be worn on or around the person's neck.

7. A wearable spectroscopic device for compositional analysis of environmental objects comprising:
a finger ring, wherein this finger ring further comprises:
a finger-encircling portion, wherein this finger-encircling portion is configured to encircle at least 70% of the circumference of a person's finger, wherein this finger-encircling portion has an interior surface which is configured to face toward the surface of the person's finger when worn, wherein this finger-encircling portion has a central proximal-to-distal axis which is defined as the straight line which most closely fits a proximal-to-distal series of centroids of cross-sections of the interior surface, and wherein proximal is defined as being closer to a person's elbow and distal is defined as being further from a person's elbow when the person's arm, hand, and fingers are fully extended;
a light-emitting member which projects a beam of light along a proximal-to-distal vector toward an object in the person's environment, wherein this vector, or a virtual extension of this vector, is either parallel to the central proximal-to-distal axis or intersects a line which is parallel to the central proximal-to-distal axis forming a distally-opening angle whose absolute value is less than 45 degrees; and
a spectroscopic optical sensor which collects data concerning the spectrum of light which is reflected from, or has passed through, the object in the person's environment, wherein data from the spectroscopic optical sensor is used to analyze the composition of this object, and wherein this spectroscopic optic sensor is selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

8. The device in claim 7 wherein the beam of light projected by the light-emitting member is near-infrared light, infrared light, or ultra-violet light.

9. The device in claim 7 wherein the beam of light projected by the light-emitting member is white light and/or reflected ambient light.

10. The device in claim 7 wherein the beam of light projected by the light-emitting member is coherent light.

11. The device in claim 7 wherein this device further comprises a laser pointer which is moved by the person in order to direct a visible beam of coherent light toward an object in the environment in order to guide, direct, select, adjust, and/or trigger spectroscopic analysis of this object.

12. The device in claim 7 wherein the vector of the beam of light projected by the light-emitting member is automatically changed in response to detection of an object in the environment and/or changes in the location of an object in the environment.

13. The device in claim 7 wherein the vector of the beam of light projected by the light-emitting member is selected in order to direct reflected light back to the spectroscopic optical sensor from an object at a selected focal distance, wherein this selected focal distance is selected based on detection of the object at the selected distance, and wherein measurement of the object's distance is based on image analysis, reflection of light energy, reflection of radio waves, reflection of sonic energy, and/or gesture recognition.

14. The device in claim 7 wherein the vector of the beam of light emitted by the light-emitting member is varied in order to scan for objects in the environment at different distances and/or to scan a larger portion of the surface of an object in the environment.

15. The device in claim 7 wherein this device further comprises a data processing unit which at least partially processes data from the spectroscopic optical sensor.

16. The device in claim 7 wherein this device further comprises a wireless data transmitter through which the device is in wireless communication with another wearable device and/or a remote computer and wherein information concerning the composition of the environmental object is displayed by the other wearable device and/or remote computer.

17. The device in claim 7 wherein this device further comprises a motion sensor and wherein motion patterns are analyzed in order to trigger or adjust the parameters of a spectroscopic scan of an object in the environment.

18. The device in claim 17 wherein a spectroscopic scan is triggered when motion patterns indicate that a person is eating.

19. The device in claim 18 wherein the device performs multiple spectroscopic scans, at different times, while a person is eating in order to better analyze the overall composition of food with different internal layers and/or a non-uniform ingredient structure.

20. A wearable spectroscopic device for compositional analysis of environmental objects comprising:
a finger ring, wherein this finger ring further comprises:
a finger-encircling portion, wherein this finger-encircling portion is configured to encircle at least 70% of the circumference of a person's finger when worn, wherein a virtual cylinder is defined as the cylinder which most closely approximates the shape of the finger-encircling portion, wherein this finger-encircling portion has a central proximal-to-distal axis which is defined as the central longitudinal axis of the virtual cylinder;
a light-emitting member, wherein this light-emitting member projects a beam of light toward an object in the person's environment, and wherein this vector, or a virtual-extension of this vector, is either parallel to the central proximal-to-distal axis or intersects a line which is parallel to the central proximal-to-distal axis forming a distally-opening angle whose absolute value is less than 45 degrees; and
a spectroscopic optical sensor, wherein this spectroscopic optical sensor which collects data concerning the spectrum of light which is reflected from or has passed through the object in the person's environment, wherein data from the spectroscopic optical sensor is used to analyze the composition of this object, and wherein this spectroscopic optic sensor is selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer; and
a laser pointer, wherein this laser pointer projects a visible beam of coherent light toward an object in the person's environment, and wherein this beam of coherent light is used by the person to select this object for spectroscopic analysis.
